(19) United States
(12) Patent Application Publication
(10) Pub. No.: US 2007/0248945 A1
(43) Pub. Date: Oct. 25, 2007

(54) STEM CELLS DERIVED FROM UNIPARENTAL EMBRYOS AND METHODS OF USE THEREOF

(76) Inventors: Kenneth John McLaughlin, Kennett, PA (US); Sigrid Eckardt, Kennett Square, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/697,248

(22) Filed: Apr. 5, 2007

Related U.S. Application Data
(63) Continuation-in-part of application No. PCT/US05/35809, filed on Oct. 5, 2005.
(60) Provisional application No. 60/616,141, filed on Oct. 5, 2004.

(51) Int. Cl. C12N 5/08; C12Q 1/00

(52) U.S. Cl. 435/4; 435/368; 435/372

(57) ABSTRACT

Embryonic stem cells derived from uniparental embryos and methods of use thereof are disclosed.

This application is a §365 (c) continuation-in-part application of PCT/US05/35809 filed 5 Oct. 2005, which in turn claims priority to U.S. Provisional Application 60/616,141 filed 5 Oct. 2004, each of the foregoing applications is incorporated herein by reference.

Pursuant to 35 U.S.C. §202(c) it is acknowledged that the U.S. Government has certain rights in the invention described, which was made in part with funds from the National Institutes of Health, Grant Numbers 1 RO3 HD045291-01 and R01DK059380.

### FIELD OF THE INVENTION

This invention relates to the fields of cell biology and the generation of cells and tissue useful for transplantation and the treatment of disease. More specifically, the invention provides compositions and methods for reconstituting the hematopoietic system using stem cells obtained from uniparental embryos.

### BACKGROUND OF THE INVENTION

Several publications and patent documents are cited throughout the specification in order to describe the state of the art to which this invention pertains. Each of these citations is incorporated by reference herein as though set forth in full.

Production and use of human embryonic stem (ES) cells has serious ethical and legal implications as derivation of these cells requires the disaggregation of potentially viable human embryos. Moreover, if these cells were to be autologous, the embryos would need to be produced by somatic cell nuclear transfer (cloning). Diploid uniparental embryos with either two maternal or paternal genomes have very limited development on their own, but can give rise to pluripotent ES cells.

To be applicable for therapeutic use, cells of embryonic, in vitro differentiated or fetal stages need to engraft into adult recipients. When combined with normal embryos to form chimeras, uniparental cells can contribute to adult tissues. It is, however, not known whether uniparental cells can repopulate postnatal tissues, bypassing a period of fetal co-development.

Parthenogenesis, the process by which a single egg can develop without the presence of the male counterpart, is a common form of reproduction in nature. Flies, ants, lizards, snakes, fish, birds, reptiles, amphibians, honeybees, and crayfish routinely reproduce in this manner. Eutherians (placental mammals) are not capable of this form of reproduction. However, chimeras of parthenogenetic cells coupled with biparentally derived embryonic tissues can develop to term and adulthood with contribution of parthenogenetic cells to various tissues (mouse: Stevens et al., 1977; Surani et al. 1977; bovine: Boediono et al. 1991; human: Strain et al. 1995). Parthenogenetic (PG)/gynogenetic (GG) and androgenetic (AG) ES cells can be derived solely from the genetic material of either one female or male, respectively. While both maternal and paternal uniparental embryos fail early in postimplantation development^{1,14}, development to the blastocyst stage and frequency of ES cell derivation from uniparental embryos is similar to that of normal embryos^{13,15-17}. Several properties of uniparental cells including differentiation bias, severe defects and lethality conveyed by AG cells in chimeras^{5,13,15}, an in vitro propensity for transformation of AG cells¹⁸, and reduced proliferation of PG cells^{11,18,19}, could limit their ability to engraft and function normally in adult recipients.

### SUMMARY OF THE INVENTION

In accordance with the present invention, compositions and methods are provided which are useful for reconstituting human adult tissues and organ systems using pluripotent cells derived from uniparental cells in patients in need thereof. An exemplary method comprises producing a uniparental embryo and culturing said embryo under conditions which result in the formation of a blastocyst. Embryonic stem cells are isolated from said blastocyst, which are then exposed to a receptor ligand cocktail which induces differentiation of said cells into a desired cell type. The cells are then cultured for a suitable time period to generate an effective amount of cells of the desired cell type; and optionally isolated for transplantation. The uniparental embryo for use in the foregoing method is selected from the group consisting of a parthenogenetic embryo, a gynogenetic embryo or an androgenetic embryo.

The stem cells of the invention can be induced to differentiate into a variety of human cell types including, without limitation, hematopoietic cells, neuronal cells, retinal cells, adipocytes, cardiac myocytes, insulin producing cells, skeletal muscle cells, primordial germ cells and hepatic cells.

Also provided in the present invention is a method for reconstituting the hematopoietic system in a non-human mammal. An exemplary method comprises providing a uniparental embryo and culturing said embryo under conditions which result in the formation of a blastocyst. Zona free blastocysts are then plated onto feeder fibroblasts and embryonic stem cells isolated from outgrowths thereof. The ES cells so derived are then injected into blastocysts thereby producing an ES cell chimera. The chimera is then transferred into a pseudopregnant female and at least one fetus is recovered from said female. A cell suspension is then obtained from the liver of said chimeric fetus and injected into an immunocompromised animal, said cells being capable of forming all cells of the hematopoietic lineage, thereby reconstituting the hematopoietic system in said immunocompromised animal. In preferred embodiments, the uniparental embryos contain cells expressing a detectable label. Alternative methods are also disclosed for reconstitution of the hematopoietic tissues in humans which do not cell passage through a pseudopregnant female.

In yet another aspect of the invention a method for assaying modulation of gene expression due to imprinting is provided. An exemplary method comprises producing a uniparental embryo and obtaining embryonic stem cells from said embryo. The ES cells are then injected into a blastocyst, thereby creating a chimeric blastocyst. The chimeric blastocyst so created in then transferred into pseudopregnant female. Uniparental cells from said fetus are obtained and analyzed for modulation of imprinted gene expression. The method optionally further comprises assessing the methylation status of imprinted genes. In an alternative embodiment, the fetus develops post-natally and cells are harvested therefrom to assess modulation of imprinted gene expression.

Methods of using the differentiated stem cells to ameliorate certain human disease states via transplantation of an effective amount of the same into patients in need thereof are also disclosed. In preferred embodiments, the cells match the MHC of the recipient.

Finally, compositions comprising the cells differentiated from the ES cells derived from the uniparental embryos described herein in a biologically acceptable carrier are also encompassed by the present invention.

### BRIEF DESCRIPTION OF THE DRAWINGS

FIGS. 1A, 1B and 1C. Various schematic diagrams for the generation of diploid uniparental embryos are shown.

FIG. 2. Experimental design and imprinting-related phenotype of uniparental ES cell derivatives. FIG. 2a provides a schematic of the experimental design employed. Briefly, eGFP expressing ES cell lines derived from uniparental embryos produced by pronuclear transfer between zygotes were injected into host blastocysts. After embryo transfer, fetuses were recovered at 13.5 to 14.5 d.p.c., chimeras identified by GFP-fluorescence, and fetal liver from chimeras transplanted into lethally irradiated congenic recipient mice. FIG. 2b. Predominance of striated muscle in AG ES cell-derived subcutaneous tumor; FIG. 2c. Postnatal GG chimeras, GFP fluorescence in skin indicating contribution of GG cells; FIG. 2d. AG chimera with overgrowth phenotype and malformations, compared to FIG. 2e. (non-chimeric littermate); FIG. 2f. Relative expression of imprinted genes in fetal liver cells from AG, N and GG ES cell chimeras and from an eGFP transgenic normal fetus (TG). Expression levels indicated are relative to beta-actin. Each color-coded bar represents gene expression in FACS sorted eGFP positive cells isolated from single fetal livers from individual fetuses. AG1, AG2, GG1 indicate the ES cell line used for chimera generation. Left panel: Genes with bias for expression from the maternal allele, right panel: Genes with preferential expression from the paternal allele. *=No data.

FIG. 3. Multilineage reconstitution by uniparental cells. FIG. 3a. Analysis of GPI-1 isoenzymes to identify contribution of uniparental or normal ES cell derived cells to the peripheral blood of recipients. Lanes 1-3 show the GPI-1 isoenzyme dimers present in the ES cells (ES; A and B isoforms), blastocysts (B; B isoform only), and adult recipients (R; B and C isoforms), respectively. (GPI-1 forms homo- and heterodimers, such that cells containing A and B isoforms contain AA, AB and BB dimers; all dimers are indicated on the left). Lanes 4-11 show the predominance of ES cell-derived cells (A, B isoforms) in the peripheral blood of individual recipients (R) 6-8 months after transplantation of ES cell chimeric fetal liver (ES line indicated on top). FIG. 3b. Presence of uniparental and normal ES derived cells in peripheral blood of recipients over time as determined by GPI-1 analysis. The majority of recipients exhibit entirely ES cell-derived peripheral blood at 6 months post transplantation. Numbers in parentheses indicate pools of fetal livers for each cell line, with identical numbers referring to the same pool. FIG. 3c. Normal lineage contribution of uniparental cells as determined by FACS analysis of peripheral blood of representative recipient mice from each experimental group (N, AG, GG) 5-7 months post transplantation. Fluorescence intensity of GFP (marking ES cell-derived cells) indicated on x axis, fluorescence intensity of differentiation markers (B220, CD4, Ter119, Gr-1) on y-axis. Gating was based on forward-scatter and side scatter profiles typical for lymphocytes/granulocytes. No difference was detected between AG, GG and N ES cell reconstituted recipients. FIG. 3d. Summary of lineage analysis. Columns represent average values for groups of 4-8 mice. F1: B6129, not transgenic; TG: B6Osb transgenic; both are controls to demonstrate the similarity of lineage and GFP positive percentages between ES reconstituted and normal mice. Dark grey bars: % of gated cells positive for lineage marker; white bars: % of GFP positive=ES cell-derived cells within lineage positive population. One Way Analysis of Variance (ANOVA) was performed with alpha=0.050, and normality tests passed (P>0.050). P values were as follows: B220 total: P=0.087; B220GFP: P=0.126; Gr-1 total: P=0.228; Gr-1GFP: P=0.635; Ter119 total: P=0.304; Gr-1/GFP: P=0.165; CD4GFP: P=0.077. For CD4 total, Kruskal-Wallis ANOVA on ranks was applied, P=0.803.

FIG. 4. Lifespan of recipients reconstituted with N, AG and GG chimeric liver. White bar indicates age in months prior reconstitution, light grey bars represent months after reconstitution. Asterisks indicate animals that were sacrificed for experimental purposes and crosses indicate animals that died of unknown causes. Ctrl.: animals reconstituted with blastocyst only derived fetal liver (B6C3×B6 F1 blastocysts). N1: eGFP-transgenic B6129 ES cell line derived from fertilized embryo; N2: E14 (129/Ola¹).

FIG. 5. Normal maturation of T- and B-lymphocytes in mice reconstituted from cells of AG, GG and normal ES cell origin. FACS analysis of recipient mice with entirely AG, N or GG derived hematopoietic system as verified by GPI-1 analysis 8 months post reconstitution. a. Percentage of cells positive for either CD4 or CD8, and double positive for both markers in peripheral blood (left) and thymus (right). While the thymus exhibits a high percentage of double positive (immature) lymphocytes, very low levels of double positive lymphocytes are detected in the peripheral blood of control (B6129) and reconstituted animals. b. Percentage of cells positive for either B220 or IgM and double positive for both markers in peripheral blood (left) and spleen (right). The similar distribution of single and double positive lymphocytes in both organs of control and reconstituted mice indicate normal maturation of B-lymphocytes.

Columns represent the average of 2 mice (B6129, AG, GG); N represents a single animal. Gating was on nucleated viable cells, and the percentage of GFP positive cells in each lineage-marker positive population was similar between reconstituted and GFP transgenic mice.

FIG. 6. Experimental design for liver regeneration and transplantation of PGCs.

FIG. 7. Timeline for recipient conditioning, transplantation and analysis of engraftment of fetal liver transplants in adult mice with liver damage.

FIGS. 8A, 8B and 8C. Experimental outline for in vitro differentiation of ES cells into different cell type progenitors and subsequent analysis (FIG. 8A). FIG. 8B shows in vitro formation of hematopoietic progenitors by N, AG and PG ES cells. N=N line 1 (E14), AG=AG3 line (McLaughlin et al. 1997), PG=B6129F1 PG ES cell line; GG not shown. CFU-GM, colony-forming unit granulocyte-macrophage; CFU-mixed, colony forming unit containing both erythroid and granulocyte-macrophage lineages. Primitive and definitive erythroid colonies per 100,000 day 6 EB cells were 4 (N); 8 (AG); 9 (GG) and 7, 8, 19 (PG); CFU-GM per 100,000 day 6 EB cells were 5 (N), 4 (AG), 2 (GG), 2, 0, 6 (PG). FIG. 8C shows analysis of neurosphere-initiating frequency in normal neurosphere cultures. Wells that developed no neurospheres 2 weeks post seeding were scored as negative, and fractions of negative wells were plotted against the numbers of cells seeded (semi-logarithmic scale). Limiting dilution was n=2. The linear regression curve (y=e^{-(AG/GG/wt)x}) was calculated using Microsoft Excel 2003 software.

FIG. 9. Overview of tissues to be analyzed for imprinted gene expression and methylation.

FIG. 10. Microarray analysis of the expression of imprinted genes in CD3 positive splenocytes isolated from adults reconstituted from AG (AG-1, AG-2) and GG (GG-1, GG-2) cells compared to B6129 control splenocytes. Only genes with significant expression level (above threshold, flag=P, present) are listed. The first six genes on the left (dlk-1 to zac1/plagl1) are paternally expressed; all other genes are maternally expressed.

FIG. 11. Imprinted gene expression in uniparental ES cell derived CD3/GFP positive splenocytes isolated from reconstituted recipients by FACS sorting Expression levels indicated are relative to beta-actin. AG1, AG2, GG1 indicate the ES cell line of origin. Genes with bias for expression from the maternal allele are Igf2r, Ube3 and Meg3/Gtl2 (left side of panel), genes with preferential expression from the paternal allele include Impact and U2af2-rs1 (right side of panel).

FIG. 12. Conserved methylation status of the H19 differentially methylated region (DMR) in bone marrow cells of recipients with entirely uniparental-derived hematopoietic systems. Bisulfite sequencing of the 5′ upstream region of the H19 gene (pos. −4413 to −3976; see schematic representation bottom right). This region is part of the imprinting control region that regulates reciprocal allele-specific expression of the H19 and Igf2 genes. In normal tissues, the paternal allele is methylated and the maternal allele unmethylated. Bone marrow from two different recipients (=R) with entirely AG derived hematopoietic systems as determined by GPI-1 analysis (AG1 R6, AG ES line 1 recipient 6; and AG2 R3, AG ES line 2 recipient 3), and from two different animals with hematopoietic systems of GG origin (recipients 2 and 7; GG ES cell line 1) was analyzed. Each line represents a single clone. Clones derived from AG tissue exhibit a high degree of methylation, whereas clones from GG derived tissue are not methylated, indicating conservation of parent-of origin specific methylation marks.

### DETAILED DESCRIPTION OF THE INVENTION

Mammalian uniparental embryos with duplicate maternal or paternal genomes are not viable¹⁻³, but diploid uniparental embryos can form embryonic stem (ES) cells⁴⁻⁶. However, until the present invention, it was not known whether these cells could reconstitute or functionally replace adult tissues or organs. Moreover, the therapeutic applicability of uniparental cells is undetermined. Uniparental maternal (parthenogenetic/gynogenetic) and paternal (androgenetic) embryonic cells can contribute to diverse tissues in chimeras⁷⁻⁹, but their differentiation is biased^{5,10,11} and correlates with parent-of-origin dependent (imprinted) gene expression^{12,13}. Based on the limited and biased contribution of uniparental cells in chimeras with normal embryos, and the abnormal expression of imprinted genes in uniparental cells and embryos, it appeared likely that uniparental cells would have limited capacity for differentiation and proliferation subsequent to transplantation. In accordance with the present invention, we have ascertained the capacity of gynogenetic and androgenetic cells to replace adult tissue by transplanting uniparental ES cell-derived fetal liver cells into lethally irradiated adult mice. Both maternal and paternal uniparental cells conveyed long-term, multi-lineage reconstitution of the entire hematopoietic system of recipients, with no associated pathologies. The uniparental ES cells and chimeras used for transplants displayed imprinting-related phenotypes, however, uniparental hematopoietic cells recovered from adult recipients exhibited no bias in the expression of imprinted genes. We demonstrate that uniparental cells, both gynogenetic and androgenetic, can form adult repopulating hematopoietic stem cells, and establish that uniparental cells are therapeutically applicable.

The methods disclosed can be slightly modified to obtain uniparental blood cells from humans. For this purpose, human ES cells obtained using the methods disclosed herein are differentiated in vitro to form hematopoietic progenitors. See Kaufman et al. (2001) PNAS 98:10716-10721.

The methods of the present invention provide similar benefits to those of therapeutic cloning yet also possess several distinct advantages over conventionally used techniques. These are as follows:

1. Minimization of ethical concerns over the destruction of embryos that are inviable. If sperm and intact oocyte never meet, then such cells do not comprise embryos as conventionally defined;

2. Uniparental ES cells are autologous to the respective oocyte or sperm donor, and therefore minimize rejection problems associated with the use of existing human ES cell lines.

3. The present methods avoid the highly controversial practice of cloning humans to generate autologous ES cells.

4. The genomes of uniparental embryos and ES cells derived thereof are gamete-derived, and thus have been protected by germline protection mechanisms. In contrast, embryos and ES cells derived by somatic cell nuclear transfer are subject to reprogramming errors and may propagate mutations accumulated in the somatic cell genome.

5. Uniparental ES cells have propensity to differentiate predominantly into certain tissue types and may thus be more applicable for these tissue types than normal ES cells.

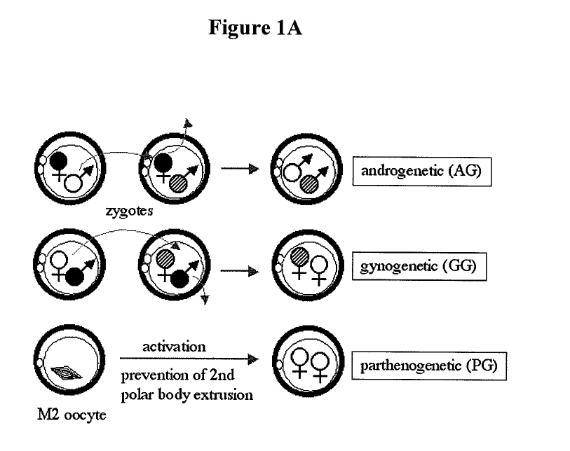

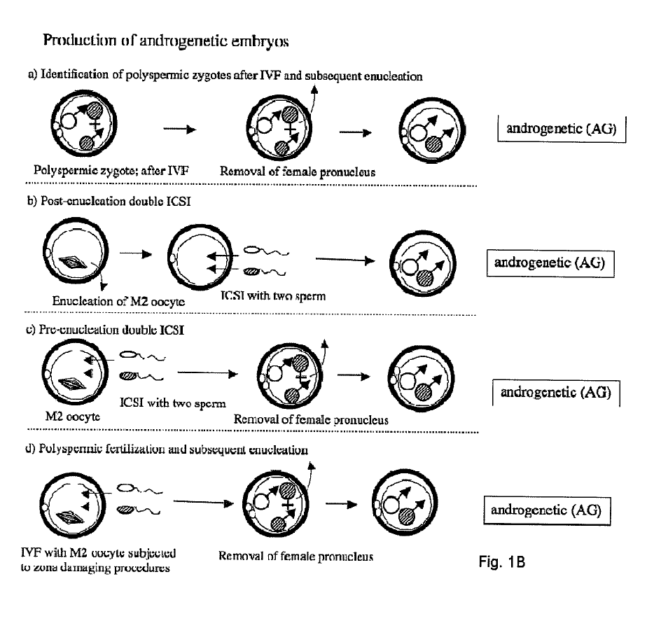

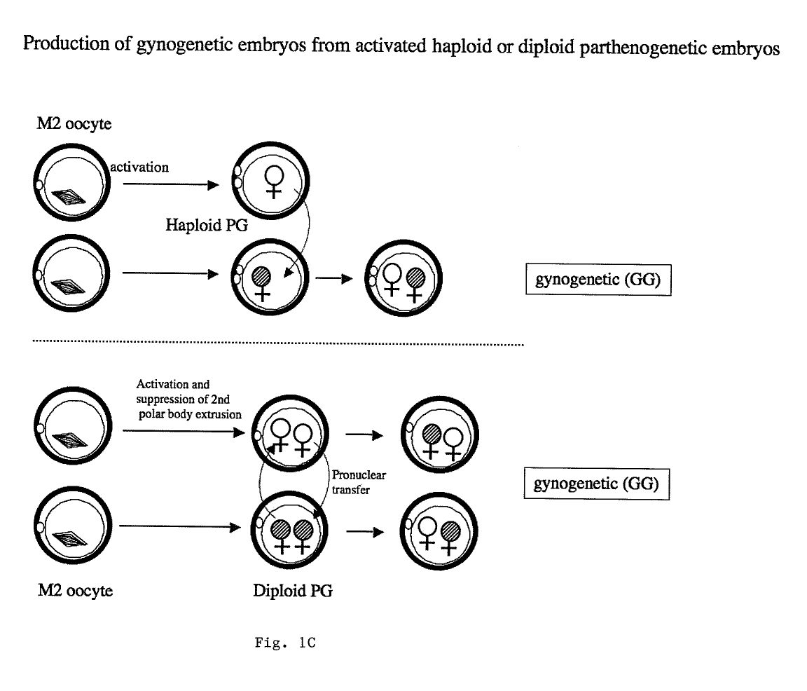

Uniparental embryos by definition, and in practice, can be generated using only the genetic material of an individual of reproductive age of either sex by either activating a female patient's oocyte (parthenogenetic; PG), or by transferring two sperm into an enucleated donor oocyte (androgenetic; AG). For mouse experimental models requiring specific genotypes, paternal and maternal uniparental embryos can be generated by the exchange of maternal and paternal pronuclei between zygotes, resulting in AG (FIG. 1 top) and gynogenetic (GG; FIG. 1 middle) embryos with two paternal and maternal genomes, respectively (McGrath and Solter, 1983). GG embryos are developmentally equivalent to PG embryos (FIG. 1 bottom) although the latter have two maternal genomes from the same oocyte (Surani and Barton, 1983).

### Androgenetic Embryos

We describe four methods that could be applied to the production of human androgenetic embryos. a) use of polyspermic embryos from IVF. b) enucleation of oocytes and treating oocytes to double ICSI (Palermo et al., 1996) (intracytoplasmic sperm injection) to introduce two sperm nuclei. c) treating oocytes to double ICSI (Palermo et al., 1996) to introduce two sperm nuclei and subsequent removal of the maternal pronucleus; d) performing zona damage procedures followed by in vitro fertilization to obtain polyspermy and subsequent removal of the maternal pronucleus. a) In human IVF, polyspermic fertilization of oocytes occurs commonly, and the resulting zygotes are typically discarded. These zygotes can however, be used to produce androgenetic embryos by removal of the maternal pronucleus. For example, zygotes with more than two pronuclei will be identified microscopically. Polyspermic zygotes will be treated with cytoskeletal and microtubule inhibitors. The female pronucleus will identified by size and proximity to the polar body and will be removed using a micropipette. Methods for the micromanipulation of human eggs have been described (Nagy, 2003) Embryos will be cultured and ES cells will be derived from these embryos as per standard protocols in the field (Pera et al., 2003). Methods for deriving embryonic stem (ES) cell lines in vitro from early preimplantation mouse embryos are well known. (See, e.g., Evans et al., Nature, 29:154-156 (1981); Martin, Proc. Natl. Acad. Sci., USA, 78:7634-7638 (1981)). ES cells can be passaged in an undifferentiated state, provided that a feeder layer of fibroblast cells (Evans et al., Id.) or a differentiation inhibiting source (Smith et al., Dev. Biol., 121:1-9 (1987)) is present Paternal-only origin of the ES cells will be confirmed by DNA fingerprinting.

In another approach, unfertilized oocytes are treated with cytoskeletal and microtubule inhibitors and the metaphase plate is removed with a micropipette. Oocytes are then treated to intracytoplasmic sperm injection with two sperm to introduce two sperm nuclei (double ICSI). ICSI is well established in the field and conditions and procedures are described, for example by (Nagy, 2003; Palermo et al., 1996). Embryos will be cultured and ES cells will be derived from these embryos as per standard protocols in the field (Pera et al., 2003). Paternal-only origin of the ES cells will be confirmed by DNA fingerprinting.

Alternatively, unfertilized oocytes are treated to intracytoplasmic sperm injection with two sperm to introduce two sperm nuclei (double ICSI). ICSI is well established in the field and conditions and procedures are described, for example by (Palermo et al., 1996). Polyspermic zygotes will be treated with cytoskeletal and microtubule inhibitors. The female pronucleus will identified by size and proximity to the polar body and will be removed using a micropipette. Embryos will be cultured and ES cells will be derived from these embryos as per standard protocols in the field (Pera et al., 2003). Paternal-only origin of the ES cells will be confirmed by DNA fingerprinting. Embryos will be cultured and ES cells will be derived from these embryos as per standard protocols in the field (Pera et al., 2003). Paternal-only origin of the ES cells will be confirmed by DNA fingerprinting.

Finally, the zona pellucida of unfertilized oocytes can be subjected to zona damaging procedures such as zona drilling and zona dissection. These methods are standard in the field and are for example described by (Nagy, 2003; Payne, 1995b). Zygotes will then be treated to IVF, either at normal or at higher sperm concentrations. (Geary and Moon, 2006) Zygotes with more than two pronuclei will be identified microscopically. Polyspermic zygotes will be treated with cytoskeletal and microtubule inhibitors. The female pronucleus will identified by size and proximity to the polar body and will be removed using a micropipette. Embryos will be cultured and ES cells will be derived from these embryos as per standard protocols in the field (Pera et al., 2003). Paternal-only origin of the ES cells will be confirmed by DNA fingerprinting.

### Parthenogenetic Embryos

Unfertilized oocytes are artificially activated by a known means for effecting artificial activation of oocytes. For human oocytes, this may be done by example as described by De Sutter and Rogers but not limited to these procedures (De Sutter et al., 1992; Rogers et al., 2004). To obtain diploidy, for example a MII oocyte is activated by a procedure that does not result in second polar extrusion. This can be done by various methods including the use of a phosphorylation inhibitor such as DMAP or by use of a microfilament inhibitor such as cytochalasin B, C or D, or a combination thereof. Thereby, cells are obtained having a diploid content of DNA of female origin which develop into an embryo having a discernible trophectoderm and inner cell mass which will not give rise to viable offspring. The inner cell mass or cells therein are used to produce pluripotent cells containing cultures which are themselves useful for making differentiated cells and tissues.

The activation of parthenogenetic oocytes has been well described in the literature. For example, Ware et al, Gamete Research, 22:265-275 (1989) teach the ability of bovine oocytes to undergo parthenogenetic activation using Ca⁺⁺, Mg⁺⁺—H⁺ ionophore (A23187) or electric shock. Also, Yang et al, Soc. Study Reprod., 46:117 (1992) teaches activation of bovine follicular oocytes using cycloheximide and electric pulse treatment. Graham C. F. in Biol. Rev., 49:399-422 (1979) describes early methods for activating parthenogenetic mammalian embryos. Further, Matthew H. Kaufmnan, in Prog in Anat., Vol. 1:1-34, ed. R. G. Harrison and R. L. Holmes, Cambridge Press, London, UK (1981) reviews parthenogenesis and methods of activation. The parthenogenetic activation of rabbit and mouse oocytes is also disclosed by Ozil, Jean Pierre, Devel., 109:117-127 (1990); Kubiak, Jacek, Devel. Biol., 136:537-545 (1989); Onodera et al, Gamete Research, 22:277-283 (1989); Siracusa et al, J. Embryol. Exp. Morphol., 43:157-166 (1978); and Szollosi et al, Chromosoma, 100:339-354 (1991). Still further, the activation of unfertilized sea urchin eggs is disclosed by Steinhardt et al, Nature, 252:41-43 (1974); and Whitaker, M., Nature, 342:636-639 (1984). Also, the parthenogenetic activation of human oocytes has been reported. (See, e.g., De Sutter et al, J. Associated Reprod. Genet., 9(4):328-336 (1992).)

### Gynogenetic Embryos

We describe two methods could be applied to the production of human gynogenetic embryos. a) use of haploid parthenogenetic embryos and subsequent transplantation of one female pronucleus to a second haploid embryo with a female pronucleus to restore diploidy. b) use of diploid parthenogenetic embryos generated by activation protocols that lead to retention of the second polar body, and subsequent exchange of one female pronucleus between two diplod embryo at the pronuclear stage. Micromanipulation will be performed in the presence of agents that enable removal of pronuclei (McGrath and Solter, 1983; Nagy, 2003).
a) Human oocytes will be activated as referred to above, and extrusion of the second polar body will not be inhibited. At the pronuclear stage, the female pronucleus from one embryo will be transferred as described in the androgenetic protocols to a second embryo of the same type and stage.
b) Human oocytes will be activated as referred to above, and extrusion of the second polar body will be prevented. At the pronuclear stage, the female pronucleus from one embryo will be transferred to a second embryo of the same type and stage, from which one of the two pronuclei has been removed.

While the present invention exemplifies hematopoietic reconstitution in the mouse, the methods should be applicable to human cells. Production of human uniparental embryos could be accomplished in several ways as described above. In preferred embodiments, the methods employed preclude the simultaneous occupation of a male and female pronucleus within an ooplast, hence technically, a zygote with a male and female genome is never formed, although certain methods do result in such simultaneous occupation. Notably, methods for hematopoietic reconstitution in humans is done entirely in vitro and does not entail passage of cells through a pseudopregnant female.

Embryonic stem cell derivation from the unparental embryos would be performed in a manner comparable to that described previously using human embryos.

As the generation of human uniparental chimeras is not acceptable or practical, the generation of uniparental cells for transplantation would be performed in vitro and will vary depending on the target tissue. Once blastocysts are obtained, stem cells may be isolated therefrom, using established techniques. See Abbondanzo et al., 1993, and Thomson 1998. The skilled person in this art area is familiar with the various culture conditions which are suitable for influencing the differentiation of stem cells down one lineage pathway or another. For reviews see Trounson, 2002, and Shufaro 2004. Also see Goldberg Cohen, 2006, Keller, 2005, and Mendendez 2005.

The differentiation of ES cells into proven and functional target cells is an extremely complex and nascent technology. AG and PG/GG cells demonstrate different and even complementary differentiation biases (Morali et al., 2000, Mann et al., 1992), although engraftment and hematopoietic reconstitution is associated with relaxation in allele-specific gene expression but not allele-specific methylation. In another aspect of the invention, methods are provided for genetically manipulating this bias to influence differentiation towards one tissue type versus another. Characterization of such biases will aid in understanding in the native differentiation pathways/factors involved and will provide targets for directing ES cell differentiation. Notably, PG/GG cells have a bias to form neural derivatives and AG cells often differentiate into mesodermal derivatives such as striated muscle. The latter would a candidate for cardiac tissue repair (infarct) and muscle atrophy diseases. PG/GG cells could be targets for (non congenic) neurodegenerative diseases.

Genomic imprinting is a parental origin-specific gene silencing that leads to differential expression of the two alleles of a gene in mammalian cells. Imprinting has attracted intense interest for several reasons. The process is by definition reversible in the germ line and may be regulated over a large genomic domain. Imprinted genes and the imprinting mechanism itself are important in human birth defects and cancer. Additionally, it has been suggested that imprinting cannot be reprogrammed without passage through the germline and thus constitutes a barrier to human embryonic stem cell transplantation. Clearly, there is a need in the art for an experimental model system which allows direct examination of allele-specific gene silencing in the dynamic process of genomic imprinting.

As mentioned, genomic imprinting is regulated by parent-specific imprinting marks that are set in the germ line, some of which involve differential methylation of regulatory regions. Our initial analyses of imprinted gene expression in adult repopulating HSC indicate that there is relaxation in the regulation of imprinted gene expression. We also observed that fetal uniparental chimeras successfully used for hematopoietic transplants displayed imprinting-related phenotypes including overgrowth and skeletal deformities in fetal AG chimeras, indicating that in fetal chimeras, the allele-specific gene expression in AG cells was retained, as also observed previously (Allen et al., 1994; Hernandez et al., 2003).

In accordance with the present invention, it has been discovered that genomic imprinting, or more specifically, the parental allele specific regulation of gene expression, is lost at some stage of the engraftment process. Thus, the present invention provides methods for ascertaining the imprinting status and the level of expression of imprinted genes in uniparental cells before and after functional engraftment, thereby elucidating the mechanism by which these cells engraft in transplants and the role of imprinting in adult tissues. In this way, the present methods facilitate the identification and characterization of the molecular factors which modulate imprinted gene expression in transplanted uniparental tissues. In preferred embodiments, imprinted gene expression patterns and methylation in tissues prior and post transplantation are determined using microarray analysis.

The following definitions are provided to facilitate an understanding of the present invention:

The term “autologous cells” as used herein refers to donor cells which are genetically compatible with the recipient.

A “hybrid cell” refers to the cell immediately formed by the fusion of a unit of cytoplasm formed from the fragmentation of an oocyte or zygote with an intact somatic or stem cell or alternatively a derivative portion of said somatic or stem cell, containing the nucleus.

The term “karyoplast” refers to a fragment of a cell containing a nucleus. A karyoplast is surrounded by a membrane, either the nuclear membrane or other natural or artificial membrane.

“Multipotent” implies that a cell is capable, through its progeny, of giving rise to several different cell types found in the adult animal.

“Pluripotent” implies that a cell is capable, through its progeny, of giving rise to all the cell types which comprise the adult animal including the germ cells. Both embryonic stem and embryonic germ cells are pluripotent cells under this definition.

A “reconstructed embryo” is an embryo made by the fusion of an enucleated oocyte with a donor somatic or embryonic stem (ES) or embryonic germ (EG) cell; alternatively, the donor cell nucleus can be isolated and injected into the oocyte. In yet another approach chromatin or nuclear DNA may be injected into the oocyte to create the reconstructed embryo.

The term “transgenic” animal or cell refers to animals or cells whose genome has been subject to technical intervention including the addition, removal, or modification of genetic information. The term “chimeric” refers an entity such as an individual, organ, cell, nucleic acid or part thereof consisting of regions derived from entities of diverse genetic constitution.

A “zygote” refers to a fertilized one-cell embryo.

The term “totipotent” as used herein can refer to a cell that gives rise to a live born animal. The term “totipotent” can also refer to a cell that gives rise to all of the cells in a particular animal. A totipotent cell can give rise to all of the cells of an animal when it is utilized in a procedure for developing an embryo from one or more nuclear transfer steps. Totipotent cells may also be used to generate incomplete animals such as those useful for organ harvesting, e.g., having genetic modifications to eliminate growth of an organ or appendage by manipulation of a homeotic gene. Additionally, genetic modification rendering oocytes, such as those derived from ES cells, incapable of development in utero would ensure that human derived ES cells could not be used to derive human oocytes for reproduction and only for applications such as therapeutic cloning.

A “blastocyst” is a preimplantation embryo that develops from a morula. A blastocyst has an outer layer called the trophoblast that is required for implantation into the uterine epithelium and an inner cell mass that contains the embryonic stem cells and will give rise to the embryo proper. A blastocyst normally contains a blastocoel or a blastocoelic cavity.

The term “follicle” refers to a more or less spherical mass of cells sometimes forming a cavity. Ovarian follicles comprise egg cells and the corona radiata.

The term “cultured” as used herein in reference to cells can refer to one or more cells that are undergoing cell division or not undergoing cell division in an in vitro environment. An in vitro environment can be any medium known in the art that is suitable for maintaining cells in vitro, such as suitable liquid media or agar, for example. Specific examples of suitable in vitro environments for cell cultures are described in Culture of Animal Cells: a manual of basic techniques (3.sup.rd edition), 1994, R. I. Freshney (ed.), Wiley-Liss, Inc.; Cells: a laboratory manual (vol. 1), 1998, D. L. Spector, R. D. Goldman, L. A. Leinwand (eds.), Cold Spring Harbor Laboratory Press; and Animal Cells: culture and media, 1994, D. C. Darling, S. J. Morgan John Wiley and Sons, Ltd.

The term “cell line” as used herein can refer to cultured cells that can be passaged at least one time without terminating. The invention relates to cell lines that can be passaged indefinitely. Cell passaging is defined hereafter.

The term “suspension” as used herein can refer to cell culture conditions in which cells are not attached to a solid support. Cells proliferating in suspension can be stirred while proliferating using apparatus well known to those skilled in the art.

The term “monolayer” as used herein can refer to cells that are attached to a solid support while proliferating in suitable culture conditions. A small portion of cells proliferating in a monolayer under suitable growth conditions may be attached to cells in the monolayer but not to the solid support. Preferably less than 15% of these cells are not attached to the solid support, more preferably less than 10% of these cells are not attached to the solid support, and most preferably less than 5% of these cells are not attached to the solid support.

The term “plated” or “plating” as used herein in reference to cells can refer to establishing cell cultures in vitro. For example, cells can be diluted in cell culture media and then added to a cell culture plate, dish, or flask. Cell culture plates are commonly known to a person of ordinary skill in the art. Cells may be plated at a variety of concentrations and/or cell densities.

The term “cell plating” can also extend to the term “cell passaging.” Cells of the invention can be passaged using cell culture techniques well known to those skilled in the art. The term “cell passaging” can refer to a technique that involves the steps of (1) releasing cells from a solid support or substrate and disassociation of these cells, and (2) diluting the cells in media suitable for further cell proliferation. Cell passaging may also refer to removing a portion of liquid medium containing cultured cells and adding liquid medium to the original culture vessel to dilute the cells and allow further cell proliferation. In addition, cells may also be added to a new culture vessel which has been supplemented with medium suitable for further cell proliferation.

The term “proliferation” as used herein in reference to cells can refer to a group of cells that can increase in number over a period of time.

The term “permanent” or “immortalized” as used herein in reference to cells can refer to cells that may undergo cell division and double in cell numbers while cultured in an in vitro environment a multiple number of times until the cells terminate. A permanent cell line may double over 10 times before a significant number of cells terminate in culture. Preferably, a permanent cell line may double over 20 times or over 30 times before a significant number of cells terminate in culture. More preferably, a permanent cell line may double over 40 times or 50 times before a significant number of cells terminate in culture. Most preferably, a permanent cell line may double over 60 times before a significant number of cells die in culture.

The term “reprogramming” or “reprogrammed” as used herein may refer to materials and methods that can convert a cell into another cell having at least one differing characteristic. Additionally, “reprogramming” of a nucleus may refer to altering the expression pattern of the genome of the nucleus. Also, such materials and methods may reprogram a nucleus to convert (e.g. differentiate) a cell into another cell type that is not typically expressed during the life cycle of the former cell. For example, (1) a non-totipotent cell can be converted into a totipotent cell; (2) a precursor cell can be converted into a cell having a morphology of an embryonic germ (EG) cell; and (3) a precursor cell can be converted into a totipotent cell.

The term “isolated” as used herein can refer to a cell that is mechanically separated from another group of cells. Examples of a group of cells are a developing cell mass, a cell culture, a cell line, and an animal.

The term “fetus” as used herein can refer to a developing cell mass that has implanted into the uterine membrane of a maternal host. A fetus can include such defining features as a genital ridge, for example. A genital ridge is a feature easily identified by a person of ordinary skill in the art, and is a recognizable feature in fetuses of most animal species.

The term “fetal cell” as used herein can refer to any cell isolated from and/or has arisen from a fetus or derived from a fetus, including amniotic cells. The term “non-fetal cell” is a cell that is not derived or isolated from a fetus. The term “parturition” as used herein can refer to a time that a fetus is delivered from female recipient. A fetus can be delivered from a female recipient by abortion, c-section, or birth.

The term “primordial germ cell” as used herein can refer to a diploid precursor cell capable of becoming a germ cell. Primordial germ cells can be isolated from any tissue in a developing cell mass, and are preferably isolated from genital ridge cells of a developing cell mass. A genital ridge is a section of a developing cell mass that is well-known to a person of ordinary skill in the art.

The term “embryonic stem cell” as used herein can refer to pluripotent cells isolated from an embryo that are maintained in in vitro cell culture. Such cells are rapidly dividing cultured cells isolated from cultured embryos which retain in culture the ability to give rise, in vivo, to all the cell types which comprise the adult animal, including the germ cells. Embryonic stem cells may be cultured with or without feeder cells. Embryonic stem cells can be established from embryonic cells isolated from embryos at any stage of development, including blastocyst stage embryos and pre-blastocyst stage embryos. Embryonic stem cells may have a rounded cell morphology and may grow in rounded cell clumps on feeder layers. Embryonic stem cells are well known to a person of ordinary skill in the art. See, e.g., WO 97/37009, entitled “Cultured Inner Cell Mass Cell-Lines Derived from Ungulate Embryos,” Stice and Golueke, published Oct. 9, 1997, and Yang & Anderson, 1992, Theriogenology 38: 315-335. See, e.g., Piedrahita et al. (1998) Biol. Reprod. 58: 1321-1329; Wianny et al. (1997) Biol. Reprod. 57: 756-764; Moore & Piedrahita (1997) In Vitro Cell Biol. Anim. 33: 62-71; Moore, & Piedrahita, (1996) Mol. Reprod. Dev. 45: 139-144; Wheeler (1994) Reprod. Fert. Dev. 6: 563-568; Hochereau-de Reviers & Perreau, Reprod. Nutr. Dev. 33: 475-493; Strojek et al., (1990) Theriogenology 33: 901-903; Piedrahita et al., (1990) Theriogenology 34: 879-901; and Evans et al., (1990) Theriogenology 33: 125-129.

The term “differentiated cell” as used herein can refer to a precursor cell that has developed from an unspecialized phenotype to a specialized phenotype. For example, embryonic cells can differentiate into an epithelial cell lining the intestine. Materials and Methods of the invention can reprogram differentiated cells into totipotent cells. Differentiated cells can be isolated from a fetus or a live born animal, for example.

The term “undifferentiated cell” as used herein can refer to a precursor cell that has an unspecialized phenotype and is capable of differentiating. An example of an undifferentiated cell is a stem cell.

The term “modified nuclear DNA” as used herein can refer to a nuclear deoxyribonucleic acid sequence of a cell, embryo, fetus, or animal of the invention that has been manipulated by one or more recombinant DNA techniques. Examples of recombinant DNA techniques well known to a person of ordinary skill in the art, can include (1) inserting a DNA sequence from another organism (e.g., a human organism) into target nuclear DNA, (2) deleting one or more DNA sequences from target nuclear DNA, and (3) introducing one or more base mutations (e.g., site-directed mutations) into target nuclear DNA. Cells with modified nuclear DNA can be referred to as “transgenic cells” or “chimeric cells” for the purposes of the invention. Transgenic cells can be useful as materials for nuclear transfer cloning techniques provided herein. The phrase “modified nuclear DNA” may also encompass “heterologous or corrective nucleic acid sequence(s)” which confer a benefit to the cell, e.g., replacement of a mutated nucleic acid molecule with a nucleic acid encoding a biologically active, phenotypically normal polypeptide. The constructs utilized to generate modified nuclear DNA may optionally comprise a reporter gene encoding a detectable product.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radioimmunoassay, or by calorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

“Selectable marker” as used herein refers to a molecule that when expressed in cells renders those cells resistant to a selection agent. Nucleic acids encoding selectable markers may also comprise such elements as promoters, enhancers, translational control sequences, poly A addition signals, transcriptional termination signals and the like. Suitable selection agents include antibiotic such as kanamycin, neomycin, and hygromycin.

Methods and tools for insertion, deletion, and mutation of nuclear DNA of mammalian cells are well-known to a person of ordinary skill in the art. See, Molecular Cloning, a Laboratory Manual, 2nd Ed., 1989, Sambrook, Fritsch, and Maniatis, Cold Spring Harbor Laboratory Press; U.S. Pat. No. 5,633,067, “Method of Producing a Transgenic Bovine or Transgenic Bovine Embryo,” DeBoer et al., issued May 27, 1997; U.S. Pat. No. 5,612,205, “Homologous Recombination in Mammalian Cells,” Kay et al., issued Mar. 18, 1997; and PCT publication WO 93/22432, “Method for Identifying Transgenic Pre-Implantation Embryos”; WO 98/16630, Piedrahita & Bazer, published Apr. 23, 1998, “Methods for the Generation of Primordial Germ Cells and Transgenic Animal Species. These methods include techniques for transfecting cells with foreign DNA fragments and the proper design of the foreign DNA fragments such that they effect insertion, deletion, and/or mutation of the target DNA genome.

Any of the cell types defined herein can be altered to harbor modified nuclear DNA. For example, embryonic stem cells, embryonic germ cells, fetal cells, and any totipotent cell defined herein can be altered to harbor modified nuclear DNA. Examples of methods for modifying a target DNA genome by insertion, deletion, and/or mutation are retroviral insertion, artificial chromosome techniques, gene insertion, random insertion with tissue specific promoters, homologous recombination, gene targeting, transposable elements, and/or any other method for introducing foreign DNA. Other modification techniques well known to a person of ordinary skill in the art include deleting DNA sequences from a genome, and/or altering nuclear DNA sequences. Examples of techniques for altering nuclear DNA sequences are site-directed mutagenesis and polymerase chain reaction procedures. Therefore, the invention relates in part to mammalian cells that are simultaneously totipotent and transgenic.

The term “recombinant product” as used herein can refer to the product produced from a DNA sequence that comprises at least a portion of the modified nuclear DNA. This product can be a peptide, a polypeptide, a protein, an enzyme, an antibody, an antibody fragment, a polypeptide that binds to a regulatory element (a term described hereafter), a structural protein, an RNA molecule, and/or a ribozyme, for example. These products are well defined in the art.

The term “promoters” or “promoter” as used herein can refer to a DNA sequence that is located adjacent to a DNA sequence that encodes a recombinant product. A promoter is preferably linked operatively to an adjacent DNA sequence. A promoter typically increases an amount of recombinant product expressed from a DNA sequence as compared to an amount of the expressed recombinant product when no promoter exists. A promoter from one organism can be utilized to enhance recombinant product expression from a DNA sequence that originates from another organism. For example, a vertebrate promoter may be used for the expression of jellyfish GFP in vertebrates. In addition, one promoter element can increase an amount of recombinant products expressed for multiple DNA sequences attached in tandem. Hence, one promoter element can enhance the expression of one or more recombinant products. Multiple promoter elements are well-known to persons of ordinary skill in the art. In a preferred embodiment, the promoters of the invention drive germ line specific expression of the transgenes described herein. Such promoters include the truncated Oct4 promoter, the GCNA promoter, the c-kit promoter and the mouse Vasa-homologue protein (mvh) promoter.

The term “enhancers” or “enhancer” as used herein can refer to a DNA sequence that is located adjacent to the DNA sequence that encodes a recombinant product. Enhancer elements are typically located upstream of a promoter element or can be located downstream of or within a coding DNA sequence (e.g., a DNA sequence transcribed or translated into a recombinant product or products). Hence, an enhancer element can be located 100 base pairs, 200 base pairs, or 300 or more base pairs upstream or downstream of a DNA sequence that encodes recombinant product. Enhancer elements can increase an amount of recombinant product expressed from a DNA sequence above increased expression afforded by a promoter element. Multiple enhancer elements are readily available to persons of ordinary skill in the art.

The term “nuclear transfer” as used herein can refer to introducing a full complement of nuclear DNA from one cell to an enucleated cell (e.g. egg). Nuclear transfer methods are well known to a person of ordinary skill in the art. See, e.g., Nagashima et al. (1997) Mol. Reprod. Dev. 48: 339-343; Nagashima et al. (1992) J. Reprod. Dev. 38: 73-78; Prather et al. (1989) Biol. Reprod. 41: 414-419; Prather et al. (1990) Exp. Zool. 255: 355-358; Saito et al. (1992) Assis. Reprod. Tech. Andro. 259: 257-266; and Terlouw et al. (1992) Theriogenology 37: 309. Nuclear transfer may be accomplished by using oocytes that are not surrounded by a zona pellucida.

The term “thawing” as used herein can refer to a process of increasing the temperature of a cryopreserved cell, embryo, or portions of animals. Methods of thawing cryopreserved materials such that they are active after a thawing process are well-known to those of ordinary skill in the art.

The terms “transfected” and “transfection” as used herein refer to methods of delivering exogenous DNA into a cell. These methods involve a variety of techniques, such as treating cells with high concentrations of salt, an electric field, liposomes, polycationic micelles, or detergent, to render a host cell outer membrane or wall permeable to nucleic acid molecules of interest. These specified methods are not limiting and the invention relates to any transformation technique well known to a person of ordinary skill in the art.

The term “antibiotic” as used herein can refer to any molecule that decreases growth rates of a bacterium, yeast, fungi, mold, or other contaminants in a cell culture. Antibiotics are optional components of cell culture media. Examples of antibiotics are well known in the art. See Sigma and DIFCO catalogs.

The term “feeder cells” as used herein can refer to cells that are maintained in culture and are co-cultured with target cells. Target cells can be precursor cells, embryonic stem cells, embryonic germ cells, cultured cells, and totipotent cells, for example. Feeder cells can provide, for example, peptides, polypeptides, electrical signals, organic molecules (e.g., steroids), nucleic acid molecules, growth factors (e.g., bFGF), other factors (e.g., cytokines such as LIF and steel factor), and metabolic nutrients to target cells. Certain cells, such as embryonic germ cells, cultured cells, and totipotent cells may not require feeder cells for healthy growth. Feeder cells preferably grow in a mono-layer.

Feeder cells can be established from multiple cell types. Examples of these cell types are fetal cells, mouse cells, Buffalo rat liver cells, and oviductal cells. These examples are not meant to be limiting. Tissue samples can be broken down to establish a feeder cell line by methods well known in the art (e.g., by using a blender). Feeder cells may originate from the same or different animal species as precursor cells. Feeder cells can be established from ungulate fetal cells, mammalian fetal cells, and murine fetal cells. One or more cell types can be removed from a fetus (e.g., primordial germs cells, cells in the head region, and cells in the body cavity region) and a feeder layer can be established from those cells that have been removed or cells in the remaining dismembered fetus. When an entire fetus is utilized to establish fetal feeder cells, feeder cells (e.g., fibroblast cells) and precursor cells (e.g., primordial germ cells) can arise from the same source (e.g., one fetus).

The term “receptor ligand cocktail” as used herein can refer to a mixture of one or more receptor ligands. A receptor ligand can refer to any molecule that binds to a receptor protein located on the outside or the inside of a cell. Receptor ligands can be selected from molecules of the cytokine family of ligands, neurotrophin family of ligands, growth factor family of ligands, and mitogen family of ligands. Examples of receptor/ligand pairs are: epidermal growth factor receptor/epidermal growth factor, insulin receptor/insulin, cAMP-dependent protein kinase/cAMP, growth hormone receptor/growth hormone, and steroid receptor/steroid. It has been shown that certain receptors exhibit cross-reactivity. For example, heterologous receptors, such as insulin-like growth factor receptor 1 (IGFR1) and insulin-like growth factor receptor 2 (IGFR2) can both bind IGF1. When a receptor ligand cocktail comprises a stimulus, the receptor ligand cocktail can be introduced to a precursor cell in a variety of manners known to a person of ordinary skill in the art.

The term “cytokine” as used herein refers to a large family of receptor ligands. The cytokine family of receptor ligands includes such members as leukemia inhibitor factor (LIF); cardiotrophin 1 (CT-1); ciliary neurotrophic factor (CNTF); stem cell factor (SCF), which is also known as Steel factor; oncostatin M (OSM); and any member of the interleukin (IL) family, including IL-6, IL-1, and IL-12. The teachings of the invention do not require the mechanical addition of steel factor (also known as stem cell factor in the art) for the conversion of precursor cells into totipotent cells.

The term “cloned” as used herein can refer to a cell, embryonic cell, fetal cell, and/or animal cell having a nuclear DNA sequence that is substantially similar or identical to a nuclear DNA sequence of another cell, embryonic cell, fetal cell, and/or animal cell. A cloned embryo can arise from one nuclear transfer process, or alternatively, a cloned embryo can arise from a cloning process that includes at least one re-cloning step. Additionally, a clone embryo may arise by the splitting of an embryo (e.g. the formation of monozygotic twins). If a cloned embryo arises from a cloning procedure that includes at least one re-cloning step, then the cloned embryo can indirectly arise from a totipotent cell since the re-cloning step can utilize embryonic cells isolated from an embryo that arose from a totipotent cell.

The term “implanting” refers to impregnating a female animal with an embryo as described herein. Implanting techniques are well known by the skilled person. See, e.g., Polge & Day, 1982, “Embryo transplantation and preservation,” Control of Pig Reproduction, D J A Cole and G R Foxcroft, eds., London, UK, Butterworths, pp. 227-291; Gordon, 1997, “Embryo transfer and associated techniques in pigs,” Controlled reproduction in pigs (Gordon, ed), CAB International, Wallingford UK, pp 164-182; and Kojima, 1998, “Embryo transfer,” Manual of pig embryo transfer Procedures, National Livestock Breeding Center, Japanese Society for Development of Swine Technology, pp 76-79. The embryo may be allowed to develop in utero, or alternatively, the fetus may be removed from the uterine environment before parturition.

The term “nuclear donor” as used herein can refer to a cell or a nucleus from a cell that is translocated into a nuclear acceptor. A nuclear donor may be a totipotent mammalian cell. In addition, a nuclear donor may be any cell described herein, including, but not limited to a non-embryonic cell, a non-fetal cell, a differentiated cell, a somatic cell, an embryonic cell, a fetal cell, an embryonic stem cell, a primordial germ cell, a genital ridge cell, a cumulus cell, an amniotic cell, a fetal fibroblast cell, a hepatacyte, an embryonic germ cell, an adult cell, a cell isolated from an asynchronous population of cells, and a cell isolated from a synchronized population of cells where the synchronous population is not arrested in the G0 stage of the cell cycle. A nuclear donor cell can also be a cell that has differentiated from an embryonic stem cell. See, e.g., Piedrahita et al. (1998) Biol. Reprod 58: 1321-1329; Shim et al. (1997) Biol. Reprod. 57: 1089-1095; Tsung et al. (1995) Shih Yen Sheng Wu Hsueh Pao 28: 173-189; and Wheeler (1994) Reprod Fertil. Dev. 6: 563-568. In addition, a nuclear donor may be a cell that was previously frozen or cryopreserved.

The term “enucleated oocyte” as used herein can refer to an oocyte which has had its nucleus or its chromosomes removed. Typically, a needle can be placed into an oocyte and the nucleus and/or chromosomes can be aspirated into the needle. The needle can be removed from the oocyte without rupturing the plasma membrane. This enucleation technique is well known to a person of ordinary skill in the art. See, e.g., U.S. Pat. No. 4,994,384; U.S. Pat. No. 5,057,420; and Willadsen, 1986, Nature 320:63-65. If the oocyte is obtained in an immature state (e.g. as with current bovine techniques), an enucleated oocyte is prepared from an oocyte that has been matured for greater than 24 hours, preferably matured for greater than 36 hours, more preferably matured for greater than 48 hours, and most preferably matured for about 53 hours.

The term “injection” as used herein in reference to embryos, can refer to perforation of an oocyte with a needle, and insertion of a nuclear donor in the needle into the oocyte. In preferred embodiments, a nuclear donor may be injected into the cytoplasm of an oocyte or in the perivitelline space of an oocyte. For a direct injection approach to nuclear transfer, a whole cell may be injected into an oocyte, or alternatively, nuclear DNA or a nucleus isolated from a cell may be injected into an oocyte. Such an isolated nucleus may be surrounded by nuclear membrane only, or the isolated nucleus may be surrounded by nuclear membrane and plasma membrane in any proportion. An oocyte may be pre-treated by any of a variety of known techniques which improve the survival rate of the oocyte after nuclear injection, such as by incubating the oocyte in sucrose prior to injection of a nuclear donor.

The term “electrical pulses or fusion” as used herein can refer to subjecting a karyoplast and recipient oocyte to an electric current. For nuclear transfer, a nuclear donor and recipient oocyte can be aligned between electrodes and subjected to electrical current. Electrical current can be alternating current or direct current. Electrical current can be delivered to cells for a variety of different times as one pulse or as multiple pulses. Cells are typically cultured in a suitable medium for delivery of electrical pulses. Examples of electrical pulse conditions utilized for nuclear transfer are well known in the art.

The term “fusion agent” as used herein can refer to any compound or biological organism that can increase the probability that portions of plasma membranes from different cells will fuse when a nuclear donor is placed adjacent to a recipient oocyte. In preferred embodiments fusion agents are selected from the group consisting of polyethylene glycol (PEG), trypsin, dimethylsulfoxide (DMSO), lectins, agglutinin, viruses, and Sendai virus. These examples are not meant to be limiting and other fusion agents known in the art are applicable and included herein.

The term “activation” can refer to any materials and methods useful for stimulating a cell to divide before, during, and after a nuclear transfer step. The term “cell” as used in the previous sentence can refer to an oocyte, a nuclear donor, and an early stage embryo. These types of cells may require stimulation in order to divide after nuclear transfer has occurred. The invention pertains to any activation materials and methods known to a person of ordinary skill in the art.

Examples of components that are useful for non-electrical activation include ethanol; inositol trisphosphate (IP3); divalent ions (e.g., addition of Ca2+ and/or Sr2+); microtubule inhibitors (e.g., cytochalasin B); ionophores for divalent ions (e.g., the a3+ ionophore ionomycin); protein kinase inhibitors (e.g., 6-dimethylaminopurine (DMAP)); protein synthesis inhibitors (e.g., cyclohexamide); phorbol esters such as phorbol 12-myristate 13-acetate (PMA); and thapsigargin. It is also known that temperature change and mechanical techniques are also useful for non-electrical activation. The invention includes any activation techniques known in the art. See, e.g., U.S. Pat. No. 5,496,720, entitled “Parthenogenic Oocyte Activation,” issued on Mar. 5, 1996, Susko-Parrish et al., and Wakayama et al. (1998) Nature 394: 369-374. When ionomycin and DMAP are utilized for non-electrical activation, ionomycin and DMAP may be introduced to cells simultaneously or in a step-wise addition, the latter being a preferred mode.

“In vitro fertilization” or “IVF” as used herein refers to a specialized technique by which an ovum is fertilized by sperm outside the body, with the resulting embryo later implanted in the uterus for gestation.

The phrase “intracytoplasmic sperm injection” or “ICSI” involves injection of single sperm into a single egg in order to effect fertilization.

As mentioned above, the present invention may be employed to generate target tissues for therapeutic applications. Once embryonic stem cells have been obtained from the uniparental embryos described herein, they may be cultured to differentiate into particular tissue types. Tissues currently being developed from embryonic stem cells include, but are not limited to: hematopoietic lineages (Keller, 1993, Kyba 2002, Kaufman 2002, Wang 2005 J. Exp Med, Wang 2005 Exp Hem); heart muscle (Klug, M. G. et al., J. Clin. Invest. (1996) 98:216-224; review Boheler, K. R. et al., Cir. Res. (2002) 91:189-201, Mummery 2002), pancreas (Soria, B. et al., Diabetes (2000) 49:1-6; Ramiya, V. K. et al., Nature Med. (2000) 6:278-282), liver (Ishii et al. 2005), nervous tissue (Bjorkland, A., Novaritis Found. Symp. (2000) 231:7-15; Lee, S. H. et al., Nature Biotechnology, (2000) 18:675-679; Kim, J. H. et al., Nature (2002) 418:50-56; Liour et al, 2005), endothelial cells (Liersch et al., 2005; McCloskey et al. 2005), renal cells (Kobayashi et al, 2005). Furthermore, differentiation protocols for large-scale generation of ES-derived cells are being developed (Schroeder et al, 2005). Protocols for the differentiation of certain tissue types from stem cells are described in further detail below.

Neuronal Cells

Parkinson's disease is caused by the loss of midbrain neurons that synthesize the neurotransmitter dopamine. Delivery of dopamine-synthesizing neurons to the midbrain should alleviate the symptoms of the disease by restoring dopamine production. Stem cells obtained using the methods of the invention may be differentiated into dopamine-synthesizing neurons utilizing the protocols set forth below. (Lee, S. H. et al., Nature Biotechnology, (2000) 18:675-679; Kim, J. H. et al., Nature (2002) 418:50-56).

Various methods for neuronal differentiation of mouse and human ES cells have been described. Du et al describe methods for mouse and human ES cells and refer to individual publications. (Du and Zhang, 2004). Sonntag et al describe the current methodology of differentiating human ES cells as neural replacement tissue, with an emphasis on neurodegenerative diseases. (Sonntag and Sanchez-Pernaute, 2006).

Examples of protocols include but are not limited to:

Dopaminergic neurons. ES cells of several species have been successfully directed to form dopaminergic neurons in vitro (Cibelli et al., 2002; Kawasaki et al., 2000; Kim et al., 2002; Kim et al., 2003). The protocol by Lee et al (Lee et al., 2000) includes the following steps: Following EB formation, cells expressing the intermediate filament nestin are enriched, expanded, and subsequently cultured in medium supplied with a cytokine mix of human fibroblast growth factor (FGF) basic, mouse FGF-8b, and mouse sonic hedgehog amino-terminal peptide (Shh-N), supporting DA differentiation. Dopaminergic-like neurons are identified using immunostaining for tyrosine hydroxylase and neuronal class IIIβ tubulin.

Cerebellar Neurons. Salero and Hatten (Salero and Hatten, 2007) describe the differentiation of murine ES cells into mature granule neurons by sequential treatment with secreted factors WNT1, FGF8 and RA, and induction with BMP6/7 and GDF7 as well as culture in glial-conditioned medium.

Barberi et al (Barberi et al., 2003) describe the selective differentiation of mouse ES cells into neural stem cells, astrocytes, oligodendrocytes or neurons, and further (by defining culture conditions) into forebrain, midbrain, hindbrain and spinal cord identity.

In a murine model, mouse ES cells were first transfected by electroporation with a plasmid expressing nuclear receptor related-1 (Nurr1), a transcription factor that has a role in the differentiation of midbrain precursors into dopamine neurons and a plasmid encoding neomycin resistance. Transfected clones (Nurr1 ES cells) were then subsequently isolated by culturing the cells in G418. The Nurr1 ES cells were then expanded under cultures which prevented differentiation (e.g., growth on gelatin-coated tissue culture plates in the presence of 1,400 U/ml-I of leukemia inhibitory factor (LIF; GIBCO/BRL, Grand Island, N.Y.) in ES cell medium consisting of knockout Dulbecco's minimal essential medium (GIBCO/BRL) supplemented with 15% FCS, 100 mM MEM nonessential amino acids, 0.55 mM 2-mercaptoethanol, L-glutamine, and antibiotics (all from GIBCO/BRL)). To induce EB formation, the cells were dissociated into a single-cell suspension by 0.05% trypsin and 0.04% EDTA in PBS and plated onto nonadherent bacterial culture dishes at a density of 2-2.5×10⁴ cells/cm² in the medium described above. The EBs were formed for four days and then plated onto adhesive tissue culture surface in the ES cell medium. After 24 hours of culture, selection of nestin-positive cells, a marker of developmental neuorns, was initiated by replacing the ES cell medium by serum-free Dulbecco's modified Eagle's medium (DMEM)/F12 (1: 1) supplemented with insulin (5 μg/ml), transferrin (50 μg/ml), selenium chloride (30 nM), and fibronectin (5 μg/ml) (ITSFn) medium. After 6-10 days of selection, expansion of nestin-positive cells was initiated. Specifically, the cells were dissociated by 0.05% trypsin/0.04% EDTA, and plated on tissue culture plastic or glass coverslips at a concentration of 1.5-2×10⁵ cells/cm² in N2 medium modified (described in Johe, K. et al., Genes Dev. (1996) 10:3129-3140), and supplemented with 1 μg/ml of laminin and 10 ng/ml of bFGF (R&D Systems, Minneapolis, Minn.) in the presence of murine N-terminal fragment of sonic hedgehog (SHH; 500 ng/ml) and murine fibroblast growth factor (FGF) 8 isoform b (100 ng/ml; both from R&D Systems). Before cell plating, dishes and coverslips were precoated with polyornithine (15 mg/ml) and laminin (1 μg/ml, both from Becton Dickinson Labware, Bedford, Mass.). Nestin-positive cells were again expanded for six days. The medium was changed every two days. Differentiation was induced by removal of basic fibroblast growth factor (bFGF). The differentiation medium consisted of N2 medium supplemented with laminin (1 mg/ml) in the presence of cAMP (1 μM) and ascorbic acid (200 μM, both from Sigma, St. Louis, Mo.). The cells were incubated under differentiation conditions for 6-15 days.

78% of Nurr1 ES cells were found to be induced into dopamine-synthesizing, tyrosine hydroxylase (TH, a rate limiting enzyme in the biosynthesis of dopamine) positive neurons by the method set forth above. The resultant neurons were further characterized to express a variety of midbrain-specific markers such as Ptx3 and Engrailed 1 (En-1). The dopamine-synthesizing, TH⁺ cells were also grafted into a rodent model of Parkinson's disease and were shown to extend axons, form functional synaptic connections, perform electrophysiological functions expected of neurons, innervate the striatum, and improve motor asymmetry.

Based on differentiation studies in vitro (differentiation and neurospheres) and in vivo (teratomas), PG/GG cells have a propensity to form neuroectodermal cell types, and our data suggest that such cells form neural stem cells more efficiently.

Ocular Cells/Retina

Haruta describes current methods for reproducible and efficient differentiation methods for the generation of ocular cells, including lens cells, retinal neurons, and retinal pigment epithelial (RPE) cells from ES cells (Haruta, 2005). Zhao et al describe a method to differentiate ES cells into retinal neurons. (Zhao et al., 2002).

Heart Muscle

The loss of cardiomyocytes from adult mammalian hearts is irreversible and leads to diminished heart function. Methods have been developed in which ES cells are employed as a renewable source of donor cardiomyocytes for cardiac engraftment (Klug, M. G. et al., J. Clin. Invest. (1996) 98:216-224).

Cardiac development in vitro has been well described for murine and human ES cells. Caspi and Gepstein (Caspi and Gepstein, 2006) summarize the techniques used for cardiac development of human ES cells, the potential for therapy and refer to publications with detail on the methodology. In an approach of tissue engineering, Caspi et al (Caspi et al., 2007) demonstrate that vascularized cardiac muscle can be produced from human ES cells by culturing ES cell derivatives (ES cell derived cardiomyocytes in co-culture with ES cell or umbilical vein derived endothelial cells and embryonic fibroblasts) on biodegradable scaffolds. Various protocols for the differentiation of murine ES cells into cardiomyocytes have been described, including the methods by Boheler (Boheler et al., 2002) and Kawai (Kawai et al., 2004). Fukuda and Yuasa (Fukuda and Yuasa, 2006) give an overview and reference several current methods.

In a previously described method, ES cells were first transfected by electroporation with a plasmid expressing the neomycin resistance gene from an α-cardiac myosin heavy chain promoter and expressing the hygromycin resistance gene under the control of the phosphoglycerate kinase (pGK) promoter. Transfected clones were selected by growth in the presence of hygromycin (200 μg/ml; Calbiochem-Novabiochem). Transfected ES cells were maintained in the undifferentiated state by culturing in high glucose DMEM containing 10% fetal bovine serum (FBS), 1% nonessential amino acids, and 0.1 mM 2-mercaptoethanol. The medium was supplemented to a final concentration of 100 U/ml with conditioned medium containing recombinant LIF.

To induce differentiation, 2×10⁶ freshly dissociated transfected ES cells were plated onto a 100-mm bacterial Petri dish containing 10 ml of DMEM lacking supplemental LIF. After 3 days in suspension culture, the resulting EBs were plated onto plastic 100-mm cell culture dishes and allowed to attach. Regions of cardiogenesis were readily identified by the presence of spontaneous contractile activity. For cardiomyocyte selection, the differentiated cultures were grown for 8 days in the presence of G418 (200 μg/ml; GIBCO/BRL). Cultures of selected ES-derived cardiomyocytes were digested with trypsin and the resulting single cell preparation washed three times with DMEM and directly injected into the ventricular myocardium of adult mice.

The culture obtained by this method after G418 selection is approximately 99% pure for cardiomyocytes based on immunofluorescence for myosin. The obtained cardiomyocytes contained well-defined myofibers and intercalated discs and were observed to couple juxtaposed cells consistent with the observation that adjacent cells exhibit synchronous contractile activity. Importantly, the selected cardiomyocytes were capable of forming stable intercardiac grafts with the engrafted cells aligned and tightly juxtaposed with host cardiomyocytes.

Insulin-Producing Cells

An ideal treatment for diabetes is the restoration of β-cell function or mimicking the insulin secretory pattern of these cells. Insulin-secreting cells derived from ES cells have been generated by the following method and have been shown to be capable of normalizing blood glucose levels in a diabetic mouse model (Soria, B. et al., Diabetes (2000) 49:1-6).

Several different approaches to generate insulin-secreting pancreatic islet-like cells from murine ES cells in vitro have been reported (Blyszczuk et al., 2003; Blyszczuk and Wobus, 2004; Hansson et al., 2004; Lumelsky et al., 2001; Rajagopal et al., 2003; Soria et al., 2000) (Miyazaki et al., 2004; Sipione et al., 2004). The basic protocol consists of the following steps: EB are grown in a medium containing insulin, transferrin, selenium, glutamine and fibronecting. After 4 days, nestin-positive cells are enriched from EBs and expanded by plating onto poly-L-ornithine/laminin or poly-D-lysine/laminin in N2 medium supplemented with insulin, transferrin, progesterone, putrescine and selenite and with bFGF and epidermal growth factors for further 7 days. Pancreatic differentiation is then achieved by culture in N2 medium supplemented with nicotinamide for 13-19 days.

Methods for the differentiation of human ES cells into pancreatic tissue are similar and are summarized by Trounson (Trounson, 2007) and by Gangaram-Panday et al (Gangaram-Panday et al., 2007) with references to publications of current protocols.

In other earlier described methods, ES cells were transfected by electroporation with a plasmid expressing β-gal under the control of the human insulin regulatory region and expressing the hygromycin resistance gene under the control of the pGK promoter. Transfected clones were selected by growth in the presence of hygromycin (200 μg/ml; Calbiochem-Novabiochem). Transfected ES cells were maintained in the undifferentiated state by culturing in high glucose Dulbecco's modified Eagle's medium (DMEM) containing 10% fetal bovine serum (FBS), 1% nonessential amino acids, 0.1 mM 2-mercaptoethanol, 1 mM sodium pyruvate, 100 IU/ml penicillin, and 0.1 mg/ml streptomycin. The medium was supplemented to a final concentration of 100 U/ml with conditioned medium containing recombinant LIF.

To induce differentiation to an insulin-secreting cell line, 2×10⁶ hygromycin-resistant ES cells were plated onto a 100-mm bacterial Petri dish and cultured in DMEM lacking supplemental LIF. After 8-10 days in suspension culture, the resulting EBs were plated onto plastic 100-mm cell culture dishes and allowed to attach for 5-8 days. For ES Ins/β-gal selection, the differentiated cultures were grown in the same medium in the presence of 200 μg/ml G418. For final differentiation and maturation, the resulting clones were trypsinized and plated on a 100-mm bacterial Petri dish and grown for 14 days in DMEM supplemented with 200 μg/ml G418 and 10 mM nicotinamide (Sigma), a form of Vitamin B3 that may preserve and improve beta cell function. Finally, the resulting clusters were cultured for 5 days in RPMI 1640 media supplemented with 10% FBS, 10 mM nicotinamide, 200 μg/ml G418, 100 IU/ml penicillin, 0.1 mg/ml streptomycin, and low glucose (5.6 mM).

For cell implantation, ES-derived insulin-secreting cells were washed and resuspended in RPMI 1640 media supplemented with 10% FBS, 10 mM nicotinamide, 100 IU/ml penicillin, 0.1 mg/ml streptomycin, and 5.6 mM glucose at 5×10⁶ cells/ml. The mice to receive the implantation of ES-derived insulin-secreting cells were male Swiss albino mice that had diabetic conditions induced by a single intraperitoneal injection of streptozotocin (STZ, Sigma) at 200 mg/kg body weight in citrate buffer. 1×10⁶ cells were injected into the spleen of mice under anesthesia.

The ES-derived insulin-secreting cells produced from this method produced a similar profile of insulin production in response to increasing levels of glucose to that observed in mouse pancreatic islets. Significantly, implantation of the ES-derived insulin-secreting cells led to the correction of the hyperglycemia within the diabetic mouse, minimized the weight loss experienced by the mice injected with STZ, and lowered glucose levels after meal challenges and glucose challenges better than untreated diabetic mice and similar to control nondiabetic mice.

Hepatic Cells/Liver

Mouse cells can be induced to undergo differentiation into hepatic cells that can be transplanted into models of liver damage. One method to derive hepatic cells from ES cells uses a serum free, chemically defined medium in combination with ES cells that had been transfected with a GFP reporter under the control of the Albumin enhancer/promoter (Heo et al., 2006). Hepatic precursor cells expressing GFP were detectable after 7 days of differentiation, induced by culture of ES cells in hanging drops for 5 days and subsequent plating on collagen IV coated plates in a chemically defined medium. After 28 days in culture, about 30% of cells expressed GFP and had hepatocyte-like morphology and gene expression. Using FACS sorting, GFP expressing cells were purified and transplanted into a mouse model with liver injury. ES cell derived cells engrafted and proliferated normally, and also formed biliary ephithelial cells.

A different therapeutic approach to substitute damaged liver function with ES cell derived was taken by Soto-Gutierrez (Soto-Gutierrez et al., 2006). ES cells were differentiated into hepatocytes by the following steps: 1. Culture in suspension for two days to induce EB formation; 2. transfer into a flask containing with a poly-amino-urethane coated polytetrafluoroethylene fabric that allows cell adhesion and culture for 3 days in the presence of FGF-2 and activin A; 3. co-culture with inactivated (mitomycin C treated) conditionally immortalized human liver non-parenchymal cells for 8 days in medium supplemented with DMSO and dHGF and for 3 days in medium containing dexamethasone. These cells were transfected with a GFP reporter under the control of the Albumin enhancer/promoter, and GFP expressing cells purified by FACS sorting. These cells were functional hepatocytes as shown by gene expression, in vitro function in metabolic assays, and function similar to primary hepatocytes when transplanted into 90% hepatectomized mice in a subcutaneously implanted bioartificial liver support. Such bioartificial liver support (BAL) devices contain active hepatocytes. The cells in the device remove toxins from the blood and help the recovery of hepatic function by supplying physiologically active molecules. The use of BAL devices has been described by (Chen et al., 1997; Demetriou et al., 2004).

In further studies, Shirahasi et al report the differentiation of human ES cells into albumin expressing cells, demonstrating that these cells can commit to hepatocyte lineage in vitro. Methods are as described in (Shirahashi et al., 2004).

Hematopoietic Cells

Olsen et al (Olsen et al., 2006) describe various approaches for the generation of hematopoietic lineages from embryonic stem cells and cite publications that describe these methods in detail. These include the in vitro production of specific hematopoietic lineages such as erythroid cells, mast cells, T- and B-lymphocytes, NK cells and other lineages. A well established protocol is the two-step hematopoietic differentiation method described by Keller (Keller et al., 1993; Kennedy and Keller, 2003).

Tian et al describe hematopoietic differentiation of human ES cells on S17 stromal cells and their subsequent successful engraftment in a mouse model (NOD/Scid) (Tian et al., 2006). Conditions for the in vitro differentiation of human ES cells into a phenotype similar to somatic hematopoietic stem cells are described by Wang et al (Wang et al., 2005), who also achieved engraftment of these cells in a mouse model.

Blood Vessels/Endothelial Cells

Feraud et al summarize the advances in various methods to induce blood vessel formation of ES cells in vitro and refer to publications describing the methods (Feraud and Vittet, 2003). Vascular like cells have been derived from ES cell cultures, form capillary like structures in vitro and can integrate into in vivo structures (Yamashita et al., 2000). Undifferentiated mouse ES cells are plated into methylcellulose medium containing pre-screened FBS and angiogenic cytokines for the formation of embryoid bodies (EBs) that contain endothelial precursors. EBs harvested after 11 days of culture are subcultured in a collagen-based matrix containing the same cytokines for the development of endothelial outgrowths (Choi et al., 1998; Feraud and Vittet, 2003). Zou et al. demonstrate that microvascular tubes generated from ES cells are capable of grafting onto E9-day embryo hearts and sustaining the flow of blood cells as verified by eGFP-expressing blood cells within non-eGFP ES cell-derived microvascular tubes (Zhou and Gallicano, 2006).

Upon formation of three-dimensional embryoid bodies (EB), human ES cells spontaneously differentiate into various cell types, including hematopoietic (Kaufman et al., 2001) and endothelial cells (Levenberg et al., 2002). Wang et al describe an efficient method to derive transplantable endothelial from human ES cells that bypasses EB formation (Wang et al., 2007). ES cells are placed on mouse embryonic fibroblasts in differentiation medium without supplementation of growth factors. After 10 days of differentiation, cells expressing the marker CD34 (hematopoietic and endothelial progenitor) were enriched by magnetic bead sorting. Enriched cells were capable of both hematopoietic and endothelial differentation. When placed into endothelial growth medium containing VEGF and bFGF, CD34 positive cells differentiated into adherent cells expressing endothelial markers (CD31, VE-Cadherin, endoglin, CD31, VEGF receptor 2, Tie-2, EphB4 and ephrin B2). These cells were transplanted in a tissue-engineered vessel model in SCID mice using co-transplantation with the mouse mesenchymal precursor cell line 10T1/2, and formed functional vessels that remained stable after 150 days.

Cartilage and Bone

Methods for the generation of chondrocytes from embryonic stem cells have been described for mouse and human ES cells. Kramer et al (Kramer et al., 2006; Kramer et al., 2003) describe a method for the in vitro differentiation of mouse ES cells based on EB induction in hanging drops and subsequent suspension culture in differentiation medium. Kawaguchi et al show that treatment of EB with retinoic acid can lead to mesenchymal commitment such that subsequent treatment with BMB4 leads to an osteogenic phenotype whereas TGF-beta 3 exposure induces chondrogenic differentiation (Kawaguchi et al., 2005). Hwang et al further demonstrate that culture in three dimensions by encapsulating EB cells in PEG hydrogels and in medium supplied with growth factors (TGF-β1) stimulates formation of chondrogenic phenotypes from mouse ES cells (Hwang et al., 2006).

For human ES cells, Barberi (Barberi et al., 2005) have established a method to derive mesenchymal precursor cells in vitro by co-culture of hES cells with the murine stromal cell line OP9 in the presence of 20% fetal bovine serum in alpha MEM medium. After 40 days of co-culture, a subpopulation of the differentiated ES cells expressed the marker CD73 and other markers characteristic for mesenchymal stem cells. When plated into specific culture conditions, these ES cell derived mesenchymal precursors formed adipocytes, chondrocytes (pellet culture system), osteogenic cells (in presence of beta-glycerolsulfate) and skeletal muscle (either by long-term culture for 21 days, or by culture in conditioned medium from the myoblastic cell line C2C12 or direct co-culture with this myoblastic cell line (Barberi et al., 2005).

Adipocyte Differentiation

Dani et al describe the differentiation of murine ES cells into adipocytes (Dani et al., 1997). This protocol involves the following steps. The cells are cultured in hanging drops of ES medium without LIF for 2 days. (approximately 1000-2000 cells in 20 μl drop). The cells are then switch into suspension culture in ES medium with 10⁻⁷ M all-trans retinoic acid without LIF for 3 days. The resultant embryonic bodies are then washed in ES medium, and cultured in suspension in ES medium for 2 days. Embryoid bodies are transferred into gelatin coated 24-well plates at 1 EB/well. The cells are cultured in differentiation medium for 20 days with medium changes approximately every 2-3 days. Differentiation medium comprises feeder medium (10% FCS) supplemented with 85 nM insulin (Sigma, 16634) and 2 nM triiodothyronine (Sigma, T 5516).

The confirm that adipocyes were isolated Oil Red O staining was performed (Rosen et al., 1999). Using this procedure, we have differentiated AG, GG and PG ES cell lines into adipocytes, demonstrating that all three uniparental cell types can form these cells. Such cells are useful for certain cosmetic procedures.

### Delivery of Differentiated or Genetically Modified Stem Cells for the Treatment of Disease

Methods for correction of autosomal dominant diseases, e.g. diseases that are caused by only one defective allele are also possible using the cells of the present invention. Uniparental ES cells are ideal for this as they are derived from gametes, and for one, one half of the gametes of a sick person will have a normal allele. Secondly, depending on the location of the allele, recombination could increase this. Thus, a subset of parthenogenetic ES cells should be normal, as should a subset of AG and GG cells derived from someone with the disease. Such an approach should affect gene repair without actual gene therapy in the sense of genome modification. Thus, the uniparental, disease-allele free ES cells could then be used perform tissue replacement in the patient from which they were isolated.

Examples of such diseases include, without limitation, Achondroplasia, Alexander disease, Antithrombin deficiency, Charcot-Marie-Tooth Syndrome, Ectrodactyly Cleft Chin, Ehlers-Danlos Syndrome, Familial hypercholesterolemia, Facioscapulohumeral muscular dystrophy, FOXP2 Gene, Hereditary hemorrhagic telangiectasia (Osler-Weber-Rendu Syndrome), Hereditary multiple exostoses, Hereditary spherocytosis, Huntington's Disease, Lactose Intolerance, Mandibulofacial dysostosis, Marfan Syndrome, Neurofibromatosis, Osteogenesis Imperfecta, Pfeiffer syndrome, Polycystic Kidney Disease, Treacher Collins syndrome, Tuberous Sclerosis, and Von Hippel-Lindau disease.

Another aspect of the invention entails methods for delivering differentiated cells obtained from the stem cells described herein to a patient in need thereof for the treatment of disease. One approach to treat such conditions is to transplant the differentiated cells directly into the patient. The transplanted material, in order to be clinically safe and effective, must (1) be non-immunogenic, non-thrombogenic, bio-stable, and completely non-toxic to cells and tissues of the host, (2) maintain cell viability for an extended period of time, (3) permit free passage of nutrients, secretagogues (a substance that stimulates secretion), and cell products, (4) facilitate surgical implantation and cell reseeding, and (5) be easily fixed in place and, likewise, removed.

Cell encapsulation methods have been used to isolate cells while allowing the release of desired biological materials. Two techniques have been used, microencapsulation and macroencapsulation. Typically, in microencapsulation, the cells are sequestered in a small permselective spherical container, whereas in macroencapsulation the cells are entrapped in a larger non-spherical membrane.

Lim, U.S. Pat. Nos. 4,409,331 and 4,352,883, discloses the use of microencapsulation methods to produce biological materials generated by cells in vitro, wherein the capsules have varying permeabilities depending upon the biological materials of interest being produced. Wu et al, Int. J. Pancreatology, 3:91-100 (1988), disclose the transplantation of insulin-producing, microencapsulated pancreatic islets into diabetic rats. Aebischer et al., Biomaterials, 12:50-55 (1991), disclose the macroencapsulation of dopamine-secreting cells.

An implantable permselective macrocapsule is described for use in cell therapy. The macrocapsule comprises a core comprising living cells that are capable of secreting a selected biologically active product or of providing a selected biological function and an external jacket which surrounds the core. The jacket comprises a biocompatible material that is substantially free of the encapsulated cells and has a nominal molecular weight cutoff sufficient to retain the cells within said macrocapsule. In some embodiments the nominal molecular weight cutoff of the macrocapsule is below the molecular weight of detrimental viruses which may be shed from said living cells. See U.S. Pat. No. 5,955,095.

Sittinger et al. describe a method by which cell tissue, particularly cartilage, is made available in a configuration which is favorable for implantation. Cells are applied to an absorbable support structure and are subsequently implanted together with it, such that a three-dimensional, preformed support structure is fashioned, having a stable shape and corresponding to the desired form of the implant, with an interior cavity, from a material having a cohesive inner surface and low volume such as a nonwoven polymeric material, for example; cells are introduced into the interior cavity of the support structure; and the support structure containing the cells is perfused with a nutrient solution such that the nutrient solution flows through the support structure until an intercellular matrix which binds the cells together has at least partially formed, thereby constituting the specified implant with a stable three-dimensional support structure having the desired shape. See U.S. Pat. No. 5,891,455.

Methods for the transplantation of fetal tissues and differentiated stem cells into recipient tissues and cells have also been previously described. For transplantation of cells in general, see for example, Sakai et al. (1999) J. Thorac. Cardiovascular Surg. 118:715-725 (cardiac cells); Radtke et al. (2004) Arch Opthalmol. 122:1159-65 (retinal cells); Mendez et al. (2005) Brain 128:1498-510 (neuronal cells); Nowak et al. (2005) Gut 54:972-979 (liver).

Compositions suitable for delivery of therapeutic cell types to patients have also been described. See for example, U.S. Pat. RE39542 which discloses methods for producing an agarose coated, agarose-collagen cell macrobead so produced; an agarose coated, gel-foam cell macrobead; and a agarose coated, agarose cell macrobead. In a preferred embodiment, the cells are secretory pancreatic islet cells.

Bioartificial implants and methods for their manufacture and use are described in U.S. Pat. No. 6,165,225, particularly bioartificial pancreases. In particular, the implants are thin sheets which enclose cells, which are completely biocompatible over extended periods of time and thus do not induce fibrosis. The high-density-cell-containing thin sheets are preferably completely retrievable, and have dimensions allowing maintenance of optimal tissue viability through rapid diffusion of nutrients and oxygen and also allowing rapid changes in the secretion rate of insulin and/or other bioactive agents in response to changing physiology. Implantations of living cells, tissue, drugs, medicines and/or enzymes, contained in the bioartificial implants may be made to treat and/or prevent disease.

U.S. Pat. No. 6,224,894 is directed to cross-linked polyesters of pyromellitic anhydride and a polyhydroxy compound containing more than two hydroxyl groups which are highly water absorbent, and biodegradable, water swellable. The polyesters disclosed are useful for the manufacture of medical devices such as prosthetic implants, supports for cell cultures or as wound dressings (e.g. as a debriding agent).

From the foregoing, it is clear that a variety of strategies exist for delivery of the differentiated cells obtained from the methods disclosed herein into patients. The skilled artisan is aware of these strategies and can discern appropriate therapeutic approaches based on the particular cell type to be delivered.

The following examples are provided to illustrate certain embodiments of the invention. They are not intended to limit the scope of the invention in any way.

### EXAMPLE I

### Hematopoietic Reconstitution by Uniparental Cells

The materials and methods set forth below are provided to facilitate the practice of the present invention.

ES cell lines and chimeras. Animals were maintained and used for experimentation according to the guidelines of the Institutional Animal Care and Use Committee of the University of Pennsylvania. AG embryos were produced by transplantation of the paternal pronuclei of zygotes from an intercross between C57BL/6NTac×C3H F1 females (Taconic #B6C3F1; abbreviated B6C3) and eGFP transgenic C57BL/6-TgN (ACTbEGFP)1Osb²¹ males (Jackson #003291; abbreviated B6Osb) into zygotes from a B6C3×129S1/SvImJ (Jackson #002448; abbreviated 129S1) intercross, from which the maternal pronuclei had been removed. GG embryos were produced by transplantation of the maternal pronuclei of zygotes from a 129S1×ICR (Taconic #ICR) intercross into zygotes from a B6Osb×ICR intercross, from which the paternal pronuclei had been removed. Embryos were cultured to the blastocyst stage in alpha-MEM (Sigma) supplemented with BSA (Pentex). Zona-free eGFP-positive blastocysts were placed on feeder fibroblasts and ES cell lines were derived from outgrowths under standard conditions. Normal (N) ES cell lines were derived from eGFP-positive blastocysts from 129S1×B6Osb intercross. Only uniparental embryos but not the donor zygotes could both be eGFP-transgenic and express the A-form of glucose-6-phosphate isomerase (GPI-1) that is distinct to the 129S1 strain (all other strains and outbred ICR males: GPI-1 bb), enabling unequivocal verification of the uniparental origin of ES cell lines. ES cell lines were karyotyped to identify chromosome number and sexed by PCR for the Zfy gene (oligonucleotides: 5′-CTCATGCTGGGACTTTGTGT-3′ and 5′-TGTGTTCTGCTTTCTTGGTG-3′; SEQ ID NO: 1).

The ability of ES cell-derived fetal liver cells to reconstitute irradiated adult recipients has been shown previously using entirely ES-cell-derived fetuses²⁹. Here, ES cell chimeras were produced by injection of ES cells into C57BL/6NTac (Taconic #B6, abbreviated B6) or B6C3×B6 hybrid blastocysts, and embryo transfer into pseudopregnant ICR females. Fetuses were recovered at 13.5 days post coitum (d.p.c.; AG) or at 14.5 d.p.c. (GG and N ES), and chimeric fetuses identified using GFP fluorescence and/or analysis of different isoforms of GPI-1. Uniparental and N ES cell lines were heterozygous for the alleles encoding the A and B electrophoretic forms of GPI-1, or homozygous for the A encoding allele (AG ES line 3, previously described in reference¹³), and blastocysts were homozygous for the allele encoding the B form, permitting detection and quantification of ES cell-derived cells by GPI-1 isoenzyme electrophoresis. Standard curves for GPI-1 analysis were obtained by mixing peripheral blood from mice carrying different Gpi-1 alleles at known ratios.

Realtime RT-PCR. The eGFP-positive cell population from fetal livers from individual midgestation chimeras, and eGFP and CD3 double positive cells from the spleens of reconstituted adult recipients were collected using a FACSVantage Sort (BD Pharmingen). Spleen cells were stained with a PE-conjugated monoclonal antibody specific for CD3 (BD Pharmingen). RNA was extracted from sorted cells using RNeasy columns (Quiagen). 80 ng of total RNA were Reverse transcribed using Dynabeads (Dynal), resulting in bead-coupled cDNA libraries30. Real-time PCR on Dynabead libraries was performed on a Roche LightCycler using LightCycler FastStart DNA Master SYBR Green I (Roche) according to the manufacturer's instructions. Oligonucleotide sequences were:

| | |
|---|---|
| Igf2r: | |
| 5′-TAGTTGCAGCTCTTTGCACG -3′; | SEQ ID NO: 2 |
| and | |
| 5′-ACAGCTCAAACCTGAAGCG -3′;; | SEQ ID NO: 3 |
| p57Kip2/Cdkn1c: | |
| 5′-TTCAGATCTGACCTCAGACCC-3′;; | SEQ ID NO: 4 |
| and | |
| 5′-AGTTCTCTTGCGCTTGGC-3′;; | SEQ ID NO: 5 |
| Meg3/Gtl2: | |
| 5′-TTGCACATTTCCTGTGGGAC-3′; | SEQ ID NO: 6 |
| and | |
| 5′-AAGCACCATGAGCCACTAGG-3′;; | SEQ ID NO: 7 |
| Dlk-1: | |
| 5′-CTGGCGGTCAATATCATCTTCC-3′;; | SEQ ID NO: 8 |
| and | |
| 5′-GAGGAAGGGGTTCTTAGATAGCG-3′; | SEQ ID NO: 9 |
| Igf2: | |
| 5′-CTAAGACTTGGATCCCAGAACC-3′ | SEQ ID NO: 10 |
| and | |
| 5′-GTTCTTCTCCTTGGGTTCTTTC-3′; | SEQ ID NO: 11 |
| Peg3: | |
| 5′-TAGTCCTGTGAAGGTGTGGG-3′ | SEQ ID NO: 12 |
| and | |
| 5′-GTAGGGATGGGTTGATTTGG-3′;; | SEQ ID NO: 13 |
| Ube3a: | |
| 5′-CACATATGATGAAGCTACGA-3′ | SEQ ID NO: 14 |
| and | |
| 5′-CACACTCCCTTCATATTCC-3′; | SEQ ID NO: 15 |
| Impact: | |
| 5′-ACGTTTCCCCATTTTACAAG-3′ | SEQ ID NO: 16 |
| and | |
| 5′-CTCTACATATGATTTTCTCTAC-3′;; | SEQ ID NO: 17 |
| U2afl-rs1: | |
| 5′-TAAGGCAGCACCACTTGGAC-3′ | SEQ ID NO: 18 |
| and | |
| 5′-TAAGGCAGCACCACTTGGAC-3′; | SEQ ID NO: 19 |
| beta-actin: | |
| 5′-GATATCGCTGCGCTGGTCGTC-3′ | SEQ ID NO: 20 |
| and | |
| 5′-ACGCAGCTCATTGTAGAAGGTGTGG-3′. | SEQ ID NO: 21 |

Fetal liver transplants. Single cell suspensions of fetal livers from chimeras were injected into the lateral tail vein of lethally irradiated (9.5 gy, Cesium 137 source) adult hybrid mice between B6 and 129S6/SvEv (B6129 Hybrid mice; Taconic #B6129; named B619Sv; Gpi-1 alleles bc) mice via the lateral tail vein (0.6-3×10⁶ fetal liver cells per recipient). For secondary reconstitutions, bone marrow harvested from tibiae and femora of primary recipients was injected into the lateral tail vein of lethally irradiated (9.5 gy) B6129Sv mice. Contribution of ES cell-derived cells in recipients was determined by GFP fluorescence or GPI-1 isozyme electrophoresis as described above.

Flow cytometry. Peripheral blood was obtained from the retro-orbital sinuses of recipients and white blood cells were isolated by centrifugation subsequent to lysis of red blood cells in 0.155 M ammonium chloride, 10 mM potassium bicarbonate, 0.1 mM EDTA. Spleens and thymuses of recipient mice were passed through 40 μM filters to obtain single cell suspensions. Cells were stained with phycoerythrin (PE), PE-Cy5 and biotin-conjugated monoclonal antibodies specific for lineage markers that included CD4 (L3T4), CD8 (Ly-2), CD45R/B220, Ly-6G (Gr-1), Ter119/Ly-76 and IgM (Igh-6b). Biotinylated antibodies were detected using a secondary streptavidin-PE-Cy5 conjugate. All antibodies were obtained from BD Pharmingen. Cells were analyzed on a BD LSR (BD Biosciences).

Peripheral blood hematology. Peripheral blood from the retroorbital sinuses of recipient mice was spun in microcapillary tubes (Stat-Spin) and hematocrits were read manually. Peripheral blood smears were stained with a HEMA3 Xanthene/Thiazine dye set (Fisher Scientific) and differential percentages of granulocytes, lymphocytes and monocytes analyzed by light microscopy. Total white blood cell (WBC) counts were determined using a Coulter Counter (Beckman Coulter) subsequent to dilution of blood into isotonic saline and lysis of red blood cells using zapoglobin (BD Pharmingen).

Array Analysis: Target Preparation and Hybridization. Methods were as described by the Penn MicroArray Facility website on the world wide web at med.upenn.edu/microarr/Data%20Analysis/Affymetrix/methods.htm. Spleen cells from a B6129 animal were stained with a PE-conjugated monoclonal antibody specific for CD3 (BD Pharmingen, San Diego, Calif.) and cells positive for CD3 were collected using a FACSVantage Sort (BD Pharmingen). RNA was extracted from sorted cells using RNeasy columns (Quiagen). 150 ng of total RNA were converted to first-strand cDNA using Superscript II reverse transcriptase primed by a poly(T) oligomer that incorporated the T7 promoter. Second-strand cDNA synthesis was followed by in vitro transcription for linear amplification of each transcript and incorporation of biotinylated CTP and UTP. The cRNA products were fragmented to 200 nucleotides or less, heated at 99° C. for 5 min and hybridized for 16 h at 45° C. to Affymetrix Mouse 430 version 2 microarrays. The microarrays were then washed at low (6×SSPE) and high (100 mM MES, 0.1M NaCl) stringency and stained with streptavidin-phycoerythrin. Fluorescence was amplified by adding biotinylated anti-streptavidin and an additional aliquot of streptavidin-phycoerythrin stain. A confocal scanner was used to collect fluorescence signal at 3 um resolution after excitation at 570 nm. The average signal from two sequential scans was calculated for each microarray feature.

Initial Data Analysis. Affymetrix Microarray Suite 5.0 was used to quantitate expression levels for targeted genes; default values provided by Affymetrix were applied to all analysis parameters. Border pixels were removed, and the average intensity of pixels within the 75th percentile was computed for each probe. The average of the lowest 2% of probe intensities occurring in each of 16 microarray sectors was set as background and subtracted from all features in that sector. Probe pairs were scored positive or negative for detection of the targeted sequence by comparing signals from the perfect match and mismatch probe features. The number of probe pairs meeting the default discrimination threshold (tau=0.015) was used to assign a call of absent, present or marginal for each assayed gene, and a p-value was calculated to reflect confidence in the detection call. A weighted mean of probe fluorescence (corrected for nonspecific signal by subtracting the mismatch probe value) was calculated using the One-step Tukey's Biweight Estimate. This Signal value, a relative measure of the expression level, was computed for each assayed gene. Global scaling was applied to allow comparison of gene Signals across multiple microarrays: after exclusion of the highest and lowest 2%, the average total chip Signal was calculated and used to determine what scaling factor was required to adjust the chip average to an arbitrary target of 150. All Signal values from one microarray were then multiplied by the appropriate scaling factor.

Bisulfite sequencing. Genomic DNA isolated from bone marrow cell was digested with XhoI and treated with sodium bisulfite as described (Clark et al. 1994). Bisulfite treated DNA was amplified by semi-nested PCR under standard conditions using ExTaq Hot Start Version (TaKaRa). Oligonucleotide sequences were 1401F (5′-TTTTGAATTATTATAAGGAA-3′) and 2159R (5′-ATCAAATATCC TCATAAATA-3′) for primary PCR, and 1401F and 1842R (5′-ACAAC CCTAATCTTTACACA-3′) for secondary PCR. The amplified DNA fragment was sub-cloned into pGEM-T Easy vector (Promega) for sequencing.

In vitro hematopoietic differentiation. Methylcellulose colony forming assays in medium supplied with a cytokine cocktail (M3434, StemCell Technologies) were performed after 6 days of ES cell differentiation as described (Kennedy and Keller 2003).

For transplantation, ES cells were differentiated for 6 days, then transduced with MSCVHoxB4iGFP, and cultured on OP9 stromal cells for 12 days as described (Kyba et al. 2002; Rideout et al. 2002; Kyba et al. 2003). Differentiated cells were transplanted into lethally irradiated (9.5 gy), NK depleted (i.p. injection of Anti asialo GM1 (Wako) 24 h prior to transplant) B6129Sv mice.

Statistical analysis. One-way analysis of variance (ANOVA) and Kruskal-Wallis ANOVA on Ranks were applied using SigmaStat software.

### Results

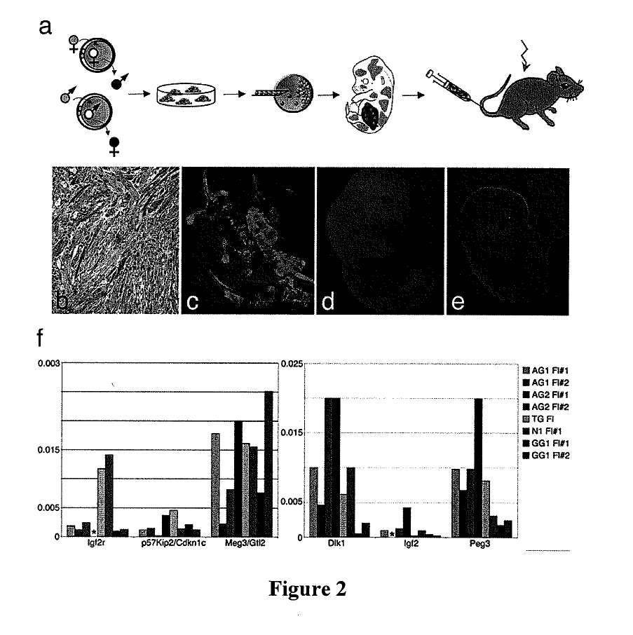

To evaluate the functionality and consequences of uniparental ES cell-derived tissue transplantation into adults, we used hematopoietic reconstitution of lethally irradiated adult mice with uniparental fetal liver cells as a model. Mammalian fetal liver contains hematopoietic stem cells (HSC) capable of long-term, multilineage reconstitution of adults²⁰. We recovered fetal liver cells from developing (13.5 to 14.5 days of gestation (days post coitum, d.p.c.)) uniparental ES cell chimeras produced by injection of ES cells into normal blastocysts (FIG. 2a). To identify ES cell-derived cells in chimeras and recipients, we derived androgenetic (AG) and biparental maternal, GG, ES cell lines from eGFP-transgenic²¹ uniparental embryos that had been generated by pronuclear transplantation^{2,3}. Two eGFP transgenic (AG1, AG2) and the characterized AG3 ES cell line (MM9=AG3¹³;), one eGFP transgenic GG (GG1), and two normal (N; derived from a fertilized embryo) ES cell lines (N1, N2) that contributed consistently to chimeras were chosen for subsequent experiments. See Table 1 below.

TABLE 1
ES cell contribution to midgestation fetuses

| | 13.5 to 14.5 d.p.c. | | | ES cell contribution | |
|---|---|---|---|---|---|
| | fetuses with ES cell contribution | | | to fetus and fetal liver | |
| | No blast. | No. chimeras/ | | Fetus | Fetal liver |
| ES line | injected | total fetuses | (%) | % | % |

Normal

| | | | | | |
|---|---|---|---|---|---|
| N line 1* | 47 | 21/28 | (75) | 50-100 | 50-100 |
| N line 2¹ | 44 | 15/18 | (83) | 40-100 | 40-100 |

AG

| | | | | | |
|---|---|---|---|---|---|
| AG line 1 | 85 | 33/63 | (52) | nd | 5-25 |
| AG line 2 | 110 | 24/57 | (42) | 15-90 | 10-60 |
| AG line 3² | 77 | 20/36 | (56) | 10-80 | 10-60 |

GG

| | | | | | |
|---|---|---|---|---|---|
| GG line 1** | 40 | 12/28 | (43) | 5-75 | 5-60 |

All ES cell lines are of BL6x129S1 genetic background, eGFP-transgenic and GPI-1 AB; except¹E14 (129/Ola) and ²MM9 (129/S1), both lines GPI-1AA.
nd, no data;
*postnatal chimeras obtained (blast n = 34; 8/8 chimeras = 100% frequency; 10-100% contrib. to peripheral blood, germline transmission)
**postnatal chimeras obtained (blast n = 45; 8/20 chimeras = 40% frequency, 5-75% contrib. to peripheral blood)

Consistent with previously reported imprinting-related phenotypes^{5,12}, all uniparental ES cell lines used formed subcutaneous tumors with characteristic tissue differentiation bias including predominance (>50%) and paucity (<5%) in the formation of striated muscle from AG and GG ES cells, respectively (FIG. 2b). The contribution of uniparental ES cells to midgestation chimeras was lower than for N ES cells, and GG chimeras with low to moderate levels of GG contribution survived postnatally (FIG. 2c). AG chimeras from all three AG lines exhibited an imprinting-related, characteristic overgrowth phenotype, various developmental defects and morbidity^{13,15} at the stage of fetal liver recovery (FIG. 2 d, f). Analysis of imprinted gene expression in uniparental-derived cells isolated from fetal livers of individual 13.5-14.5 d.p.c. AG and GG chimeras revealed parent-of-origin dependent expression bias in AG-derived fetal liver cells from two independent AG ES cell lines tested for genes that are preferentially expressed from the paternal allele (Dlk-1, Igf2 and Peg3; FIG. 2f), and lower expression levels of the maternally expressed Igf2r gene compared to controls. GG-derived fetal liver cells from chimeras exhibited bias in respect to paternally expressed genes, but not to three maternally expressed genes (Igf2r, p57Kip2/Cdkn1c and Meg3/Gtl2) that were detected at similar levels in AG and GG cells. The observed gene expression bias and chimera phenotypes are consistent with studies on differentiated uniparental ES cells and chimeras^{5,12,22,23} and indicate that imprinting in AG and GG cells in the chimeras was largely retained at the stages used for transplantation.

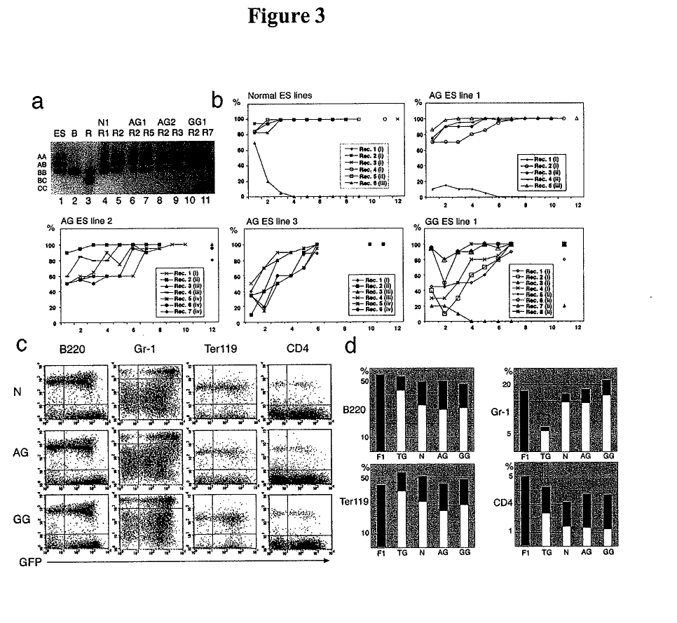

For hematopoietic reconstitution, fetal liver cells from chimeras, consisting of both blastocyst and injected ES cell derived cells, were transplanted into lethally irradiated congenic adult mice. Fetal liver transplants from AG, GG and N chimeras reconstituted recipients with similar efficacy. Contribution of ES cell- and blastocyst-derived cells to the peripheral blood of recipients determined by analysis of mouse strain-specific glucose-6-phosphate isomerase-1 isoforms (GPI-1 isozyme gel electrophoresis) revealed high levels of the ES cell-derived component in animals from all ES cell types (FIG. 3a). Regardless of the initial level of ES cell-derived cells in fetal liver transplants that varied between 10 and 60%, the contribution of ES cell-derived cells in recipients typically increased with time, and at 6-9 months post transplantation, the peripheral blood of the majority of recipients was entirely ES cell-derived (FIG. 3b). We presume that the predominance of ES cell (B6129S1 genetic background, see Methods)-over blastocyst (B6C3×B6 or B6 strains)-derived cells in recipients (B6129Sv) is due to the genetic similarity of the ES cells and recipients, since recipients receiving only blastocyst-derived fetal liver cells reconstituted entirely from these cells.

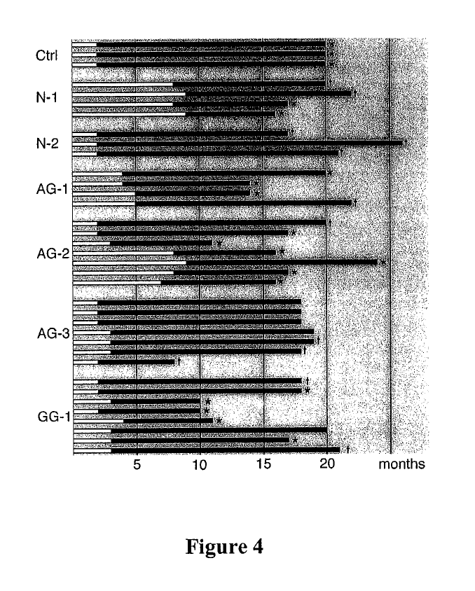

Maternal and paternal uniparental ES cells are distinct from each other and normal ES cells in their ability to differentiate into various cell types both in vitro and in Vivo^{5,12}, and cells of uniparental origin may be biased or limited in their differentiation into hematopoietic lineages. Using appropriate lineage-specific surface markers, we determined the contribution of uniparental, eGFP expressing cells to lymphoid (B220, CD4 positive), myeloid (Gr-1 positive), and erythroid (Ter119 positive) cell populations of the peripheral blood of reconstituted recipients. The percentage of peripheral blood lymphocytes positive for each lineage marker and the percentage of eGFP expressing cells in each population were similar between all groups and similar to eGFP-transgenic mice (FIG. 3c, d), indicating no bias or restriction to the differentiation of uniparental-derived cells. Steady-state hematology of peripheral blood from recipients reconstituted from AG, GG and normal ES cell-derived fetal liver was similar to non-reconstituted B6129Sv or eGFP-transgenic (B6Osb) mice (Table 2). Reconstituted recipients had a normal lifespan and no pathology associated with uniparental transplants. Also see FIG. 4.

TABLE 2
Steady-state hematology of mice reconstituted from uniparental transplants

| | Mice reconstituted with chimeric | | FL from | Control |
|---|---|---|---|---|
| Hematologic parameter | Normal ES | AG ES | GG ES | (no transplant) |
| White blood cells/μL | 8 572 ± 1 822 | 6 432 ± 1 614 | 7 215 ± 1 693 | 6 140 ± 2 345 |
| Absolute lymphocyte | 7 539 ± 1 575 | 5 327 ± 1 375 | 6 035 ± 1 559 | 5 312 ± 1 898 |
| count/μL | | | | |
| Absolute neutrophil | 813 ± 434 | 998 ± 676 | 1 028 ± 503 | 715 ± 438 |
| count/μL | | | | |
| Absolute monocyte | 220 ± 189 | 101 ± 189 | 152 ± 154 | 114 ± 61 |
| count/μL | | | | |
| Hematocrit | 46 ± 3 | 48 ± 2 | 49 ± 4 | 48 ± 3 |

Sample size consisted of 7 (normal ES, GG ES), 12 (AG ES) and 4 (no transplant) mice per group analyzed 4-7 months post transplantation. All mice appeared healthy.
No statistically significant difference was detected between values by Kruskal-Wallis One Way ANOVA on ranks. P values were as follows: White blood cells, P = 0.104; absolute lymphocyte count, P = 0.128; absolute neutrophil count, P = 0.554; absolute monocyte count, P = 0.193; hematocrit, P = 0.347.

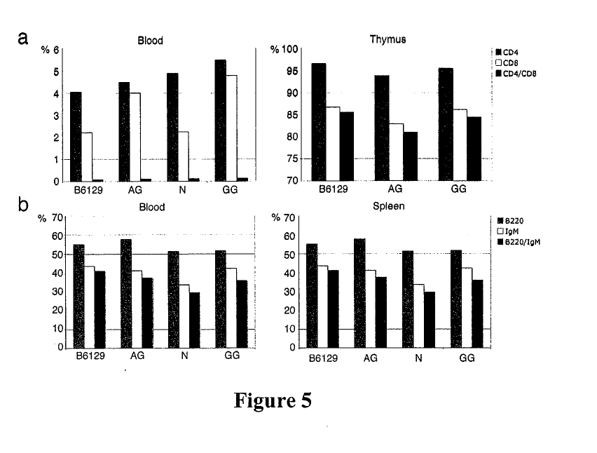

High levels of ES cell-derived cells were detected (spleen, thymus, bone marrow; see Table 3). Animals with entirely AG, GG or normal ES cell-derived hematopoietic system exhibited normal maturation of T- and B-lymphocytes, indicated by the presence of CD4 and CD8 double and single positive cells in thymus and peripheral blood, respectively, and the expression of IGM and B220 in splenic and peripheral lymphocytes (FIG. 5). Normal in vitro myeloid colony forming activity was observed for bone marrow and spleen of animals reconstituted entirely from AG, GG and normal ES cell-derived cells using a CFU-C assay in methylcellulose (not shown).

TABLE 3
Contribution of ES cell derived, GFP positive cells
to hematopoietic organs in recipient animals

| | % ES/PB | % GFP positive cells² in | | | |
|---|---|---|---|---|---|
| Recipient | (GPI-1 analysis)¹ | PWBC | Spleen | Thymus | Bone marrow |
| B6129Sv | N/A | 0.12 | 0.85 | 0.35 | 0.32 |
| B6Osb (GFP tg) | N/A | 89.76 | 76.51 | n.d. | 42.85 |
| N ES line 1 Recipient 2 | 100 | 77.06 | 58.10 | n.d. | 35.11 |
| AG ES line 1 Recipient 4 | 100 | 70.17 | 57.12 | 9.34 | 28.90 |
| AG ES line 2 Recipient 2 | 100 | 85.38 | 61.31 | 11.81 | 37.84 |
| AG ES line 2 Recipient 5 | 100 | 68.77 | 55.30 | 7.43 | 23.70 |
| GG ES line 1 Recipient 4 | 90 | 76.95 | 59.58 | 10.12 | 33.37 |
| GG ES line 1 Recipient 5 | 100 | 86.26 | 72.82 | 12.22 | 40.52 |

¹% of contribution to peripheral blood as determined by GPI-1 analysis
²of lymphocytes (gated by forward and side scatter profile) in single cell suspension of organs
PB, peripheral blood; PWBC, peripheral white blood cells;
N/A, not applicable;
n.d., not done

To establish the presence of long term repopulating HSC of uniparental origin, bone marrow from recipients with entirely ES cell-derived hematopoietic systems was transplanted into lethally irradiated secondary recipients. All recipients (19 from 4 primary donors that had been reconstituted with AG chimeric fetal liver; 10 from 3 primary donors reconstituted with GG chimeric fetal liver and 7 from 2 primary donors reconstituted with normal ES chimeric fetal liver) survived after transplantation and exhibited uniparental or normal ES cell derived donor bone marrow derived peripheral blood for more than 11 months after transplantation. See Table 4. In competitive transplantation assays of bone marrow from primary recipients mixed with bone marrow cells from congenic B6129 animals, cells of uniparental origin exhibited a stable/constant level of contribution over more than 11 months subsequent to transplantation suggesting neither a competitive disadvantage or advantage compared to normal cells.

TABLE 4
Bone marrow transplants into secondary recipients

| Recip- | | % ES derived cells in peripheral | | |
|---|---|---|---|---|
| ients | | blood at months post transplantation | | |
| (n) | Bone marrow donor | 3 | 6 | 12 |
| 4 | N ES line 1 Recipient 1 | 100 | 100 | 100 |
| 3 | N ES line 1 Recipient 2 | 100 | 100 | 100 |
| 4 | AG ES line 1 Recipient 3 | 100 | 100 | 100 |
| 2 | AG ES line 2 Recipient 4 | 100 | 100 | 100 |
| 2 | AG ES line 2 Recipient 4 | 100 | 95 | 100 |
| 4 | AG ES line 2 Recipient 1 | 100 | 100 | 100 |
| 3 | AG ES line 1 Recipient 4 | 100 | 100 | 100 |
| 2 | GG ES line 1 Recipient 6 | 100 | 100 | 100 |
| 2 | GG ES line 1 Recipient 6 | 100 | 95 | 100 |
| 3 | GG ES line 1 Recipient 4 | 100 | 100 | 100 |
| 3 | GG ES line 1 | 100 | 100 | 100 |
| | Recipient. 5 | | | |

The results of this study demonstrate that uniparental cells can functionally replace adult tissue. Furthermore, our results present a novel perspective of using androgenetic cells therapeutically. Uniparental ES cells can be derived without destruction of a potentially viable embryo, and are autologous to the donor. Parthenogenetic ES cell derivation relies on activation of unfertilized oocytes from the patient and would thus be limited to females of reproductive age, AG ES cells could be established from fertile males, using methods to facilitate multiple sperm entry or karyoplast transplantation into ooplasts¹⁶. The derivation of primate PG ES cells⁶ and derivation of human ES cells from somatic cell clones²⁴ indicates that it should be practical to produce human uniparental ES cells.

Engraftment and functionality of both AG and GG derived hematopoietic stem cells in adults demonstrates that uniparental cells can contribute to a stem cell compartment that is relevant for transplantation. Previous evidence of functional uniparental stem cells in adults existed only in the context of chimeras where contribution of uniparental cells to the germ line had been established^{8,25}, but evidence for contribution to other stem cell types has been limited or circumstantial^{19,26}. Maternal uniparental development has been demonstrated at a very low frequency by employing extensive alteration in imprinted gene expression through eliminating key loci²⁷. Our study implies that genetic manipulation need not be required for therapies using uniparental stem cells. We observe that uniparental-derived hematopoietic cells in reconstituted recipients exhibit expression of imprinted genes in a parent-of-origin independent manner. This could imply that normal expression of imprinted genes is required for reconstitution, either being required for HSC formation²⁸, or for engraftment and hematopoiesis, and would explain the absence of any imprinting related phenotypes in uniparental-derived adult hematopoietic tissue. Currently we are exploring whether this relaxation occurs before or after transplantation. Our data indicate that uniparental fetal liver tissue, in particular AG-derived cells, exhibit a parent-of origin related bias in imprinted gene expression at the time point of transplantation, suggesting that relaxation occurs in the adult recipient. It is, however, possible that uniparental adult reconstituting HSC are a subpopulation of uniparental cells with modulation in the expression of imprinted genes already at the time of transplantation.

Regardless, the formation and engraftment of normal hematopoietic tissue derived from both maternal and paternal uniparental cells establishes a precedent for transplantation of autologous tissue derived from uniparental ES cells and warrants testing for other tissue types.

Our data demonstrate that both maternal and paternal uniparental cells can engraft and functionally replace the entire adult hematopoietic system. The ability of uniparental cells to engraft into all other tissues, however, is currently being studied. While participation of uniparental cells is observed in many tissues in chimeras, the level of uniparental cell contribution is typically low, rarely more than half of the cells, and for some tissues, extremely biased depending on parental origin. Furthermore, these observations are based on co-development of uniparental and normal cells in chimeras and do not predict the outcome in direct transplants. For instance, paternal and maternal uniparental cells can contribute to the germ line of postnatal chimeras, but—particularly for AG chimeras—only at very low levels (Narasimha et al., 1997). It is unclear if the lower level of uniparental contribution to the germline, particularly in adult AG chimeras, is related to an intrinsic defect in uniparental germ cell differentiation or to effects of the chimeric environment, as is observed in the postnatal failure of chimeras with any substantial (>5%) contribution of AG cells. In order to assess the capacity of both maternal or paternal uniparental cells to form transplantable stem and precursor cells and functionally engraft into most, if not all, transplantable tissue types, we have chosen two established models of transplantation from fetal tissue, hepatic and germline tissue, to test both the level of engraftment and functionality of the engrafted tissue.

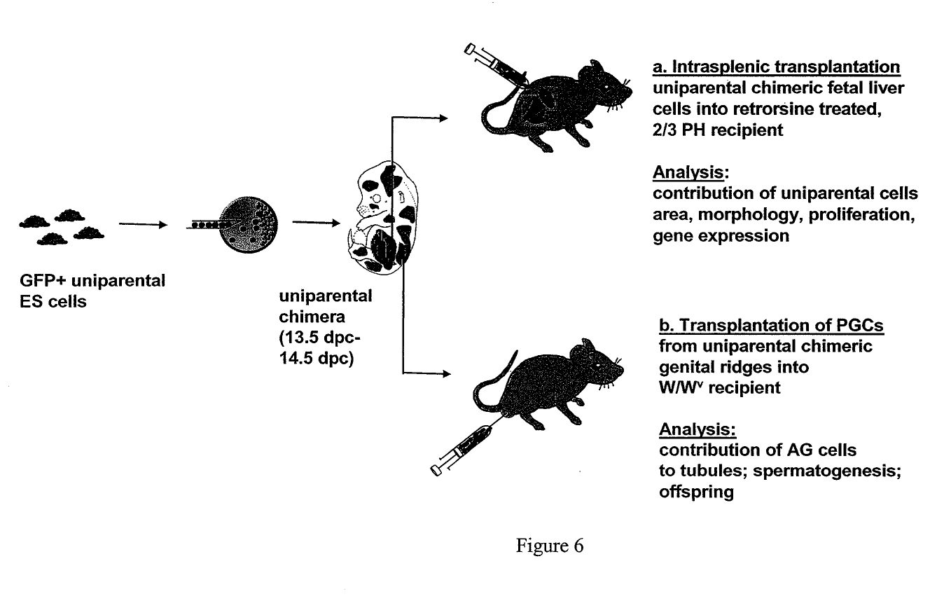

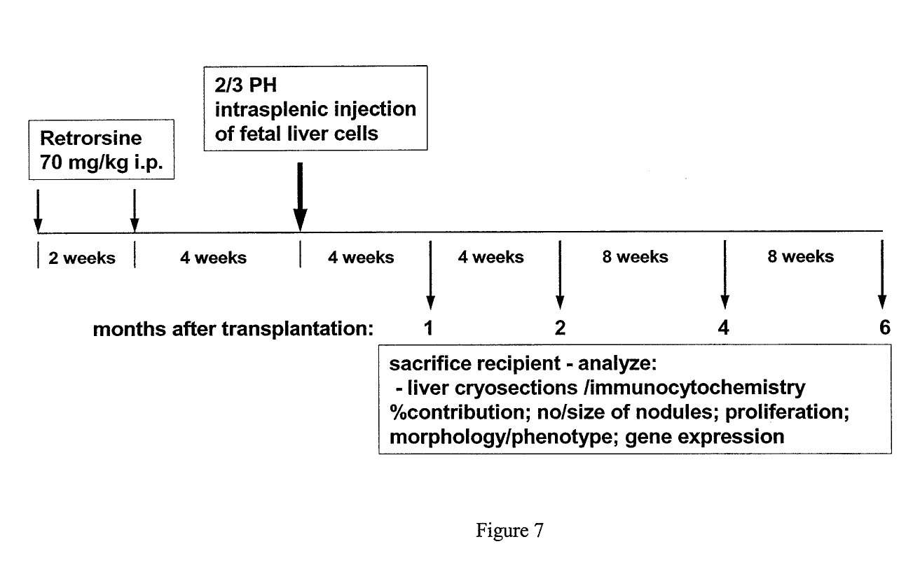

Our approach is outlined in FIG. 6. We will produce uniparental (AG, GG) and normal (N) chimeras and recover tissues at midgestation when contribution of uniparental cells to chimeras can be substantial. See above. Fetuses with ES cell contribution will be identified by GFP fluorescence, and the following tissues recovered for transplantation: Fetal liver for hepatic regeneration, and, from male AG and N chimeras, genital ridges for transplantation of primordial germ cells (PGCs). Recipient mice for liver transplants will be conditioned by drug administration and partial (⅔) hepatectomy (⅔ PH) prior to receiving fetal liver transplants from AG, GG and N chimeras by intrasplenic injection. PGCs will be injected into the testes of infertile (c-kit mutant) W/W^{v} mice. Contribution and functionality of uniparental cells in recipients will be analyzed post-transplantation by tissue-specific criteria as outlined below.

### Liver Regeneration with Uniparental Chimeric Fetal Liver Cells

Transplantation. Repopulation of the adult liver by fetal liver progenitor cells has been demonstrated in the mouse and rat using various models of liver damage, including transgene expression (Cantz et al., 2003; Sandgren et al., 1991), partial hepatectomy, and hepatotoxic drug administration (Dabeva et al., 2000; Sandhu et al., 2001). We will utilize drug administration to block endogenous hepatocyte proliferation followed by partial (⅔) hepatectomy (PH) to induce liver damage, facilitating subsequent regeneration/repopulation from the transplanted fetal liver cells. The pyrrolizine alkaloid retrorsine has been demonstrated to efficiently block the proliferation of native hepatocytes permitting proliferation of transplanted cells, and we will follow established protocols and dosages for the conditioning of mice (Guo et al., 2002; Suzuki et al., 2000). Recipient mice (B6129 F1 animals) will be conditioned prior to transplantation by two injections of retrorsine (30 mg/kg-70 mg/kg) in a two-week interval. One month after the second retrorsine injection, hepatectomy and fetal liver cell transplantation (via spleen injection) will be performed. We have established ⅔ PH in the laboratory, and attained consistent survival rates of more than 80%. Fetal liver cells from chimeras (AG, GG and N) will be harvested by collagenase digestion of dissected fetal liver and 2×10⁶ cells per recipient will be transplanted into the spleen subsequent to ⅔ PH. A small aliquot of cells will be used for semi-quantitative analysis of uniparental/N ES cell contribution to the fetal liver by GPI-1 analysis, such that the extent of ES cell derived, GFP positive, cell contribution in regenerated livers can be related to the ES derived cell contribution in the transplant.

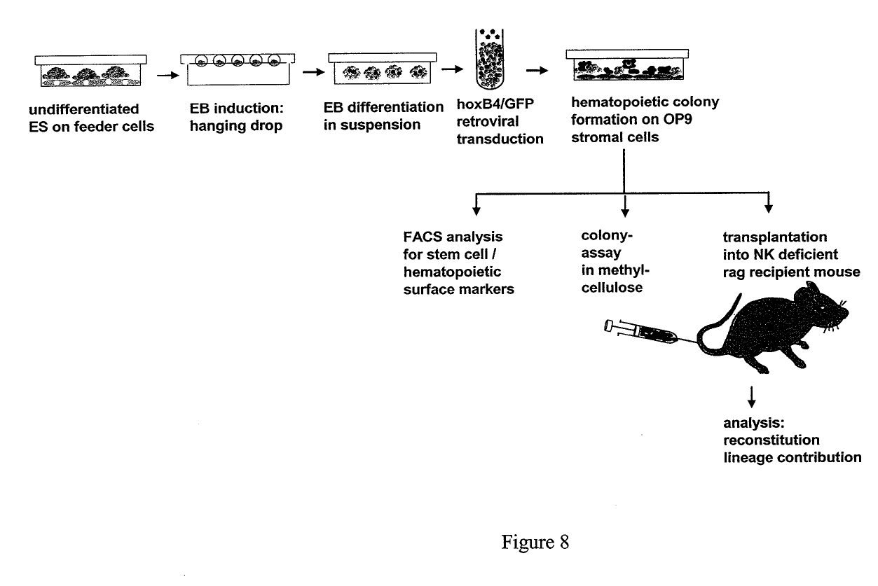

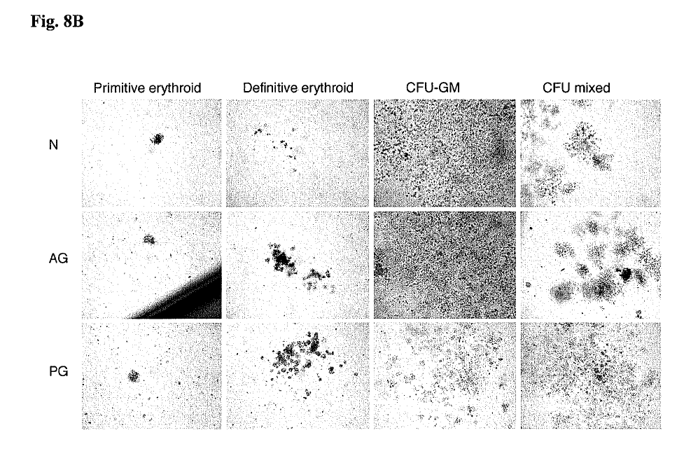

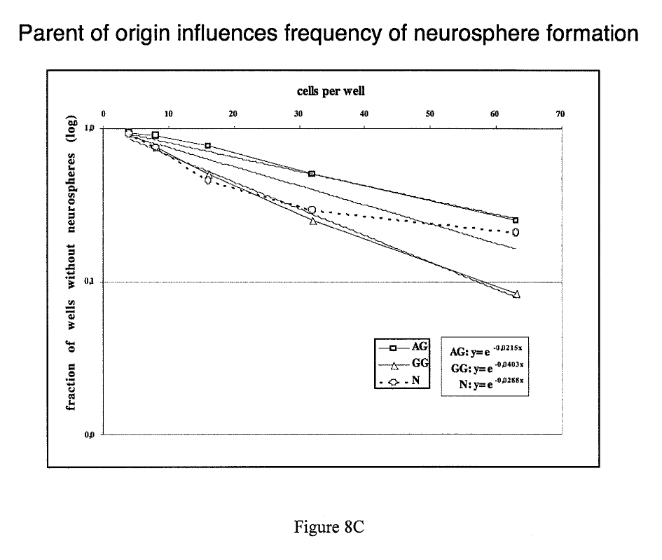

We will transplant fetal liver cells into 15 recipients per ES cell line and will include 2 ES cell lines each for AG, GG and N ES cell lines including 4 GFP-transgenic B6129 ES cell lines that have already been described above in Table 3 (AG ES lines 1 and 2; GG ES line 1 and N ES line 1). We will derive additional contributing GG and N ES cell lines to increase the number of different lines for statistical significance. For each cell line, recipients will receive transplants consisting of 4-6 different fetal liver preparations from 3 different experimental days. FIG. 8 provides a timeline for recipient conditioning, transplantation and analysis of engraftment of fetal liver transplants in adult mice with liver damage.

Analysis. Three recipients of each treatment group will be sacrificed at 1, 2, 4 and 6 months post surgery. In the mouse, differentiation of fetal liver progenitors into mature hepatocytes occurs approximately 6-8 weeks post transplantation (Cantz et al., 2003), and in retrorsine/⅔ PH treated rats, continued repopulation by transplanted fetal liver cells was detected 4-6 months post transplantation (Sandhu et al., 2001), such that repopulation can be measured and compared to controls within this time window. One hour before sacrifice, animals will receive an intraperitoneal injection of 2 mg BrdU solution to permit analysis of proliferation activity. Regenerated regions of the livers will be processed for contribution analysis by GPI-1 isozyme analysis (removal of small sample for analysis) and fixed and processed for cryosectioning. Per recipient, 20 cryosections will be scored for contribution of GFP cells. The size (cells/cluster), number (clusters/cm²) and % repopulation of GFP positive regeneration nodules will be determined and compared between groups and related to the initial level of ES cell contribution in the transplant (determined by GPI-1 analysis). Since contribution of uniparental and N ES cells to the fetal liver varies (between 10 and 90%), this correlation is essential to compare engraftment between samples. For morphological analysis of regenerated tissue (hepatocytic; ductular; mixed; endothelial) standard hematoxylin/eosin staining will be performed on adjacent sections. To verify the identity of GFP positive apparent liver parenchyma cells, selected sections will be analyzed for co-staining for GFP and the liver specific marker dipeptidyl-peptidase (DPPIV; ecto-ATPase, located on the apical membrane of mature hepatocytes; typical canalicular staining pattern; evidence for full differentiation of hepatocytes) by double immunocytochemistry with anti-mouse CD26 and anti-eGFP antibodies (BD Pharmingen and Molecular Probes, respectively). Analysis of proliferation activity (no of divisions/cluster) will be performed by immunostaining (BrdU labeling kit) and co-staining with the anti-eGFP antibody. Proliferation activity in GFP positive nodules will be calculated from the number of BrdU incorporating versus the total number of DAPI stained nuclei.

We employ established protocols for partial hepatectomy in the laboratory and observe good (>80%) survival and endogenous liver regeneration in recipients. From 15 transplanted animals per group (ES line), we expect 12 to survive, if the graft is successful, such as assumed for N=control ES cell lines. Survival of animals in AG and GG groups will also be determined. The Morphology Core (University of Pennsylvania) routinely performs cryosectioning of GFP samples, and routine immunocytochemistry will also be performed. The conditioning of recipients may be modified to substitute PH by carbon tetrachloride injection to induce acute liver damage (Guo et al., 2002). Since retrorsine administration has been established for rats and mice, we do not anticipate problems in adapting recipient conditioning protocols and in blocking endogenous liver proliferation. In the rat model of retrorsine treatment and hepatectomy, fetal liver grafts result in extensive repopulation of the liver (up to 60-80%), and we therefore expect considerable contribution from control (N ES derived) chimeric fetal liver. The ratio of engraftment of ES-derived versus blastocyst-derived cells from chimeric transplants will also be determined. In hematopoietic reconstitution experiments, we observed a preferential engraftment of ES cell (B6129) derived over blastocyst (B6) derived cells in B6129Sv hosts, presumably due to the genetic background. We may see a similar effect in liver regeneration, or we may detect GFP negative proliferative (BrdU staining) regeneration clusters that stem from blastocyst-derived cells. This will be determined by GPI-1 analysis to quantify the extent of contribution of the blastocyst component (GPI-1 BB) compared to the endogenous liver (GPI-1 BC) and ES cell derived cells (GPI-1 AB). As an alternative approach to study reconstitution from purely ES cell derived fetal liver cells, we will then apply transplantation of purified (flow sorted), ES cell derived, GFP-positive cells from fetal liver. Our preliminary studies show that in GFP transgenic animals, approximately 6-8% of fetal liver cells express the GFP transgene, consistent with a study that identified non-erythroid (TER119 negative), GFP positive cells to be 6.4% of fetal liver (Cantz et al., 2003). Depending on the percentage of ES cell contribution to the fetal liver, we have collected between 30,000 and 250,000 GFP-positive cells from single fetal livers, such that for transplantation of sorted cells, we would pool fetal livers. Quantification of liver reconstitution will be performed on liver parenchyma/hepatocytes (confirmed in their identity/function by immunocytochemistry). In rodents, the maturation of transplanted fetal liver into mature hepatocytes has been confirmed by gene expression analysis (Cantz et al., 2003; Dabeva et al., 2000). If required, we can perform additional analyses (expression of alpha-fetoprotein versus albumin using in situ hybridization) to investigate the phenotype of engrafted cells. Fetal liver progenitors can also mature into bile ducts and endothelial structures. Detection of these in uniparental grafts would confirm the presence of uniparental bipotential progenitors, and again, the phenotype of these cells can be verified with respective markers.

An alternative model to study liver regeneration is the use of transgenic recipient mice with permanent liver damage such as Urokinase plasminogen activator (uPA) transgenic mice (Sandgren et al., 1991). This mouse model, however, is currently not available from usual commercial vendors (Jackson Laboratories), but could potentially be obtained from an existing colony. The percent repopulation observed in these mice is much lower than in retrorsine treated animals due to endogenous liver regeneration (Cantz et al., 2003; Rhim et al., 1994), but would still permit analysis/comparison of uniparental versus normal cell engraftment.

### Transplantation of Primordial Germ Cells

We will transplant primordial germ cells (PGCs) from the genital ridges of 13.5 to 14.5 d.p.c. AG and N (control) chimeras into infertile recipients and examine the ability of AG versus N ES cell derived cells to repopulate the seminiferous tubules and to undergo spermatogenesis. We will use W/W^{v} mice as recipients. Homozygous dominant white spotting mutant (W) mice are congenitally infertile and lack germ cells due to a mutation in the c-kit receptor tyrosine kinase (W locus). Since homozygous W/W mice die in utero, mice carrying the less severe W^{v} allele (W/W^{v} mice; Jackson lab stock no. 100410) have been established as recipients for spermatogonial transplantation of PGCs (Chuma et al., 2005; Ohta et al., 2004). AG and N chimeras will be produced by injection of AG and N ES cells into B6 blastocysts, and will be recovered from recipients at 13.5 and 14.5 d.p.c., respectively. By dissection, genital ridges will be recovered from fetuses identified as chimeras by GFP fluorescence, and GFP fluorescence, i.e. ES cell contribution in the genital ridge confirmed. The AG (AG1, AG2) and control (N ES line 1) ES cell lines are male (XY) lines, and genital ridges will be scored for sex by morphological appearance such that only PGCs from male genital ridges are used for transplantation into male recipients. The N ES cell line 1 has exhibited frequent contribution to the germline in postnatal chimeras and thus represents a good control. Genital ridges will be dissected from the mesonephros and will be dissociated by enzymatic digestion (0.25% trypsin, 1 mM EDTA) and, after a brief wash in DMEM/10% FCS, cells will be suspended at 1×10⁸ cells/ml in injection medium (DMEM with supplements) as described (Ogawa et al., 1997). Per recipient testis, approximately 2-3 μl of cell suspension will be injected via the efferent ducts (Ogawa et al., 1997). We will transplant 10 recipients per cell line. Cell preparations from genital ridges of several fetuses per line will be pooled, and transplants performed on 4 experimental days per cell line. Depending on the cell number available on each day, we will transplant one or two testes per recipient. Cell lines include GFP transgenic, characterized lines AG lines 1 and 2; N line 1; and a second to be derived N ES line.

### Analysis

Recipient testes will be recovered 8 to 15 weeks post transplantation and analyzed by fluorescent microscopy/photography for the presence of GFP expressing clusters. Colony count, colonized area and length of colonized (GFP positive) tubules will be determined. Relevant (GFP positive, and as control, negative) areas will be cryosectioned and the extent of spermatogenesis determined in adjacent sections (GFP versus adjacent hematoxylin/eosin stained section). Sections will also be stained with fluorescence conjugated peanut agglutinin (PNA) and Hoechst for acrosomes and nuclei, respectively. For each transplant group, we will determine a) the number of testis with spermatogenesis; b) colony count/size (as described above), c) the percent of tubule (cross section) with spermatogenesis and d) functionality of sperm by derivation of offspring by mating or by intracytoplasmic sperm injection (ICSI).

For both AG ES cell lines to be tested, we have observed contribution to the genital ridges of midgestation chimeras. The control N ES cell line has resulted in germ line contribution in postnatal chimeras and represents a good control. Transplantation will be performed and the results analyzed. Transplantation of germ cells into recipient W/W^{v} mice is an established model (Chuma et al., 2005; Ogawa et al., 2000; Ohta et al., 2004). As an alternative approach, we can also transplant into non-mutant (such as B6129) recipient mice in which spermatogenesis has been ablated by treatment with the chemotherapeutic agent busulfan (Brinster et al., 2003), an approach also routinely performed.

Since approximately 20% of testis colonization is required to restore fertility, natural matings may not produce offspring. We will then perform ICSI with sperm recovered from recipient testes. Mouse ICSI is an established method (Boiani et al., 2002).

### Determination of the Ability of Uniparental ES Cells to Form Transplantable Progenitor Cells In Vitro

Our data demonstrating engraftment of uniparental cells into the hematopoietic organ is based on the transplantation of fetal stage tissue. This establishes that when co-developing with normal cells in a chimera, both maternal and paternal uniparental cells form long-term reconstituting hematopoietic stem cells that engraft in adult recipients. For therapeutic purposes, however, transplantable tissue should be derivable directly from ES cells. To date, limited evidence exists for functional engraftment of cells derived from differentiated ES cells, the notable exception being the hematopoietic system: Ectopic, inducible expression of the homeodomain protein HoxB4 in differentiating ES cells has been successfully used to promote formation of cells with a definitive hematopoietic phenotype that exhibit multilineage engraftment in adult recipients.

We will adopt a variation of this approach to test the capacity of uniparental ES cells to form transplantable hematopoietic progenitor cells in vitro. To enable the analysis of several ES cell lines, we will introduce the HoxB4 gene into differentiating ES cells using retroviral transduction, and transplant in vitro generated hematopoietic progenitors into immune-compromised recipients lacking natural killer cells. This approach does not confer the same degree of multi-lineage engraftment as demonstrated for transient (inducible) Hoxb4 expression, but has been shown to result in extensive donor-chimerism in the hematopoietic system of recipients, with predominantly myeloid engraftment (Kyba et al., 2002; Rideout et al., 2002).

The experimental outline is described in FIG. 8. In vitro differentiation of Normal (N), AG and GG ES cells will be induced using the hanging drop method to generate embryoid bodies (EB), and day 6 EB cells will be transduced with the retrovirus MSCVhoxB4iGFP directing HoxB4 and GFP expression (Kyba et al., 2002). ES cell derivatives will then be cultured on OP9 stromal cells for colony induction. This protocol is based directly on methods used in the laboratory of Dr. Michael Kyba who is providing advisory support for this Aim (see Letter of Support by Dr. M. Kyba). The formation of hematopoietic cells will be ascertained morphologically by analysis of cell surface markers. Differentiated cells will be transplanted into common gamma (γc)/Rag2 double knockout mice (Mazurier et al., 1999), a mouse model lacking natural killer (NK) cells, since the NK response may prevent engraftment of ES derived hematopoietic cells (Rideout et al., 2002).

In vitro differentiation. We will use two AG, two GG, and two N ES cell lines of 129/Ola, 129 Sv or B6129 genetic background (not GFP transgenic) for this experiment. The AG ES cell lines are MM9 and MM11 (129/Ola), previously characterized (McLaughlin et al., 1997). N ES lines are E14 and one of several 129 SvEv N ES lines that exist in the laboratory. Additional non-transgenic N and GG ES lines of B6129 F1 background will be derived and characterized. Since B6Osb animals are maintained as heterozygotes, only approximately 50% of N and GG blastocysts generated in accordance with the present methods will be GFP transgenic, such that the remaining blastocysts can be used for the derivation of non-GFP transgenic B6129 F1 ES lines. ES cells are maintained in an undifferentiated state by culture on feeder fibroblasts in the presence of leukemia inhibitory factor (LIF). To induce differentiation, cells will be cultured for two days in hanging drops in differentiation medium, without LIF and supplemented with transferrin, monothiolglycerol and ascorbic acid (Kyba et al., 2003), such that clusters of differentiating cells, so-called embryoid bodies (EB) are formed. Proliferation of EB will be achieved by suspension culture in differentiation medium for 4 more days. Day 6 EB will be harvested and spin-infected with the virus MSCVhoxB4iGFP (grown in 293T cells as described; (Kyba et al., 2002)). Expression of HoxB4 in ES cells transduced with this virus is detected by the GFP reporter, such that colonies of transduced cells can be selected for transplantation.

Subsequent to transduction, cells will be cultured on the stromal cell line OP9 (Nakano et al., 1994), in differentiation medium (IMDM, 10% FCS (tested for in vitro hematopoietic differentiation, StemCell Technologies), supplemented with murine VEGF, human TPO, human SCF and human FL as described (Kyba et al., 2002)). Colonies of semi-adherent cells will be passaged on fresh OP9 cells, and after 12-14 days in culture, cells will be assessed daily for hematopoietic phenotype by a colony forming assay in methylcellulose and by FACS analysis of lineage specific surface markers (see below). Cells for transplantation will be harvested after 14 days in culture.

In recent experiments, we ascertained hematopoietic in vitro differentiation of uniparental ES cells using established protocols for murine ES cells (Kennedy and Keller 2003). Specifically, we analyzed the formation of committed hematopoietic progenitors at day 6 of ES cell differentiation by plating ES cell derivatives at this stage in methylcellulose media containing a mix of hematopoietic cytokines that enable to evaluate the formation of primitive erythroid, definitive erythroid, megakaryocyte, macrophage and multilineage colonies. Both uniparental maternal (GG line 1; 3 parthenogenetic (PG) ES cell lines, PG lines 1-3) and a paternal (AG line 3 (MM9), previously characterized for imprinting-related phenotypes; McLaughlin et al. 1997) ES cell lines showed commitment to the same hematopoietic progenitor types in vitro as the N ES cells (N ES line 2; Hooper et al. 1987), that were consistent with previous observations (Kennedy and Keller 2003). Similar numbers of hematopoietic colonies were obtained for N, AG, GG and PG ES cell derivatives (N: 10, AG: 13, GG: 14, PG 1-3: 31, 8 and 11 colonies/100,000 cells at day 6 of differentiation).

Colony Forming Assay and Lineage Analysis.

Cells will be harvested and plated in methylcellulose suspension culture (M3434; Stem Cell Technologies) to assess the presence of hematopoietic colony forming progenitors. For derivatives of each cell line, the numbers and types of hematopoietic colonies in methylcellulose will be scored, including Colony forming unit-granulocyte, erythrocyte, macrophage, megakaryocyte (CFU-GEMM). The presence of lineage-committed versus progenitor cells in the ES derived cells as identified by specific surface markers will be analyzed by FACS (GFP versus PE-coupled antibody against respective surface marker): myeloid (Gr-1); erythroid (Ter119); lymphoid (CD4, CD8, B220); progenitor/megakaryocyte (CD41); pan-hematopoietic (CD45); stem/progenitor (Sca-1, c-kit); HSC/endothelial (CD31).

FIG. 8B shows in vitro formation of hematopoietic progenitors by N, AG and PG ES cells. N=N line 1 (E14), AG=AG3 line (McLaughlin et al. 1997), PG=B6129F1 PG ES cell line; GG not shown. CFU-GM, colony-forming unit granulocyte-macrophage; CFU-mixed, colony forming unit containing both erythroid and granulocyte-macrophage lineages. Primitive and definitive erythroid colonies per 100,000 day 6 EB cells were 4 (N); 8 (AG); 9 (GG) and 7, 8, 19 (PG); CFU-GM per 100,000 day 6 EB cells were 5 (N), 4 (AG), 2 (GG), 2, 0, 6 (PG).

After culture on OP9 stromal cells in the presence of hematopoietic cytokines, normal and parthenogenetic ES cells differentiated into semi-adherent cells with hematopoietic blast-like morphology that included cells with expression of the hematopoietic stem cell markers c-kit and Sca-1, as well as a large population of CD 41 positive cells, and minor populations positive for myeloid (Gr-1) and lymphoid (B220) differentiation markers.

Transplantation into Recipients.

In vitro derivatives of the 3 experimental groups (N, AG, GG) will be transplanted into recipient adult mice vial tail vein injection. We will use common gamma (γc)/Rag2 double knockout mice (C57BL/6J×C57BL/10SgSnAi)-[Ko]γc-)-[Ko]Rag2 (Taconic; Emerging Models Program) as recipients. In vitro differentiated cells (2×10⁶ cells/animal) will be transplanted into irradiated (9.5 gy) recipients via the lateral tail vein. We will transplant into 15 animals per ES line, resulting in 30 recipients per experimental group.

Analysis of Recipients.

Starting 2 weeks after transplantation, small amounts of peripheral blood will be taken from the tail tip of recipients, erythrocytes will be removed by lysis, and white blood cells will be analyzed by GPI-1 isoenzyme electrophoresis to determine the level contribution of ES cell derivatives to peripheral blood (GPI-1 AA versus BB of recipient). Overall contribution of ES cell derivatives to peripheral blood will be observed over 6-12 months. Lineage analysis will be performed by staining of peripheral white blood cells obtained from recipients with fluorescence-coupled antibodies directed against lineage-specific surface markers, and analysis of GFP-expressing cells within lineages by FACS. We will use the following lineage markers: B220, IgM (B-lymphocytes); CD4, CD8 (T-lymphocytes); Gr-1 (granulocytes).

Normal ES cell lines serve as experimental control, and we expect to see hematopoietic chimerism with in vitro derivatives of these cells in primary recipients. We will determine whether uniparental ES cells behave in a similar manner and the results may vary between AG and GG cells. Our choice of using constitutive HoxB4 expression (which results in predominantly myeloid contribution in recipients, with little or no lymphoid contribution) over inducible expression is based on the simplicity and feasibility of this approach. Generating ES cell lines with inducible HoxB4 expression requires several sequential targeting steps which creates problems for the analysis of several different ES cell lines such as several AG and GG in comparison to normal. Viral transduction is feasible for a number of lines, and the readout will provide information on the capacity of uniparental ES cells to form adult repopulating cells in vitro.

Neural Differentiation of Uniparental ES Cells In Vitro: Formation of Pan-Neural Progenitor Cells

To initiate studies on the in vitro neural differentiation potential of uniparental ES cells, we cultured AG, GG and normal ES cells according to a multi-step protocol that facilitates ES cell differentiation towards neuronal and glial cell types (Brustle et al., 1997). Uniparental and normal ES cell lines were grown on primary mouse feeder layers. For differentiation, ES cells were cultured under differentiation conditions (reduced FCS content, absence of LIF and embryonic feeder cells) to generate embryoid bodies. After 4 days of in vitro differentiation, embryoid bodies were plated into a medium that was supplemented with insulin, transferrin, sodiumselenite and fibronectin, in order to obtain attached embryoid bodies. After a further period of 4 days, attached embryoid bodies were dissociated using trypsin. Single cell suspensions were transferred to poly-L-ornithine coated plates, and cells were further cultured in medium supplemented with bFGF, insulin and laminin. After further 4 days of culture plastic-adherent pan-neural progenitor cells with elongated shapes developed in cultures of all three ES cell types (N, AG, GG).

Neural differentiation of uniparental ES cells in vitro: Differentiation into neuronal and glial cell types. AG, GG and normal ES-derived pan-neural progenitors cells were cultured under neural and glial differentiation conditions (neural basal medium with NeuroCult™ differentiation supplement, StemCell Technologies). β III-tubulin+neuronal and glial fibrillary acidic protein positive (GFAP+) astroglial cell types developed, with neuronal and astroglial morphology, respectively, from AG, GG and N ES cells.

Our preliminary experiments thus suggest that, similar to GG and normal ES-derived pan-neural progenitor cells, AG-derived cells grow in culture and differentiate into cells with neuronal and astroglial morphology and immunophenotype.

Isolation of Neurospheres from Chimeric Brains and Analysis of Neurosphere-Initiating Frequency.

The isolation of neurosphere-forming stem cells of AG origin from the brain of midgestation chimeras, and analysis of their differentiation potential as well as the ability to form new neurospheres (neurosphere initiating frequency) can be performed as follows. We will isolate eGFP positive cells of androgenetic origin by FACsort (flow cytometer capable of cell sorting) from the forebrains of chimeras at approximately day 14 of development (13.5 to 14.5 days post coitum=d.p.c.). To establish the feasibility of this experimental approach, we injected eGFP transgenic normal (N) ES cells into blastocysts and FACS sorted the eGFP positive fraction of fetal brains from 14.5 d.p.c. chimeras. Free-floating neurosphere cultures were established as previously described (Kirchhof et al., 2002; Schmittwolf et al., 2005). We compared the neurosphere system to a novel protocol for the culture of adherent neural stem cells (Conti et al., 2005), and found the neurosphere culture to be more robust and reproducible. The proposed experiments are therefore based on the neurosphere culture system.

To investigate the neurosphere-initiating frequency (i.e self-renewal activity), we performed limiting dilution assays by seeding 96-well-plates with graded numbers of dissociated neurosphere cells (FIG. 8C). Single cell suspensions were prepared from neurospheres by trypsinisation (passage number>6). During 2 weeks of culture, the development of new neurospheres was observed microscopically. As expected, the number of neurospheres decreased from the lowest (500 cells per well) to the highest dilution (4 cells per well). Two weeks post seeding, the number of wells with newly generated neurospheres was counted for every dilution. Using exponential regression analysis, the frequency of neurosphere-initiating cells was calculated to be 1 out of 35 (2.9%). See FIG. 8C.

### EXAMPLE 2

### Methods for Analysis of Imprinted Gene Expression

The developmental failure and defects observed in uniparental embryos and uniparental chimeras are associated with the abnormal expression of imprinted genes due to the presence of duplicate maternal or paternal alleles. The equivalence of AG and GG cells in forming adult-repopulating fetal liver HSC therefore either indicates that imprinted genes were not expressed in, or not consequential for HSC formation and differentiation, or that imprinting was relaxed. Consistent with previously reported imprinting-related phenotypes, the uniparental ES cells used to generate chimeras formed subcutaneous tumors with characteristic tissue differentiation bias including predominance (>50%) and paucity (<5%) in the formation of striated muscle from AG and GG ES cells, respectively. As expected, GG chimeras survive postnatally with substantial contribution of GG cells, while AG chimeras consistently exhibit mortality and a characteristic overgrowth phenotype at the stage of fetal liver recovery (data not shown) and have extremely low postnatal survival.

We intend to assess the effects of imprinting on gene expression and gene methylation in a variety of ways. These include gene array analysis, and bisulfite sequencing. In order to determine the relevance of both expression and methlyation patterns in many reconstituted tissues, we will establish allele-specific expression in normal tissue. Parent-of-origin specific expression is largely uncharacterized for most imprinted genes in most adult tissues. We will use hybrid mice carrying alleles with strain-specific polymorphisms (restriction, length, and single nucleotide polymorphism) that enable identification of the expressed parental allele. This is an essential control that needs to be conducted for the adult tissues. To generate F1 mice with large number of parental allele specific polymorphisms we have established a colony of JF/Ms mice (Japanese fancy mice; Mus musculus molossinus;), for which allele-specific PCR-based assays for imprinted genes, including Igf2r, Igf2, H19, impact, dlk-1, gtl3, are established. We will use F1 animals from reciprocal crosses (JF with B6 or 129) to verify allele-specific expression of imprinted genes in selected tissues. We also have preliminary data on polymorphisms for a limited number of genes between B6 and 129 that can be also used for the existing transplanted tissues and uniparental cell lines.

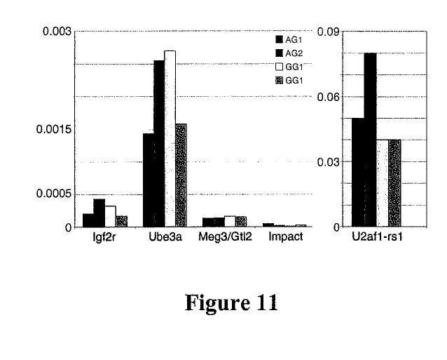

To characterize imprinted gene expression in uniparental tissue engrafted in adult recipients, we isolated eGFP/CD3-double positive splenocytes (see Table 5 below) from recipients reconstituted entirely from uniparental cells. We identified imprinted genes that are expressed in adult CD3-positive splenocytes by microarray analysis of normal CD3-positive splenocytes, and performed semi-quantitative real-time RT-PCR on uniparental-derived cells. No expression bias was detected for the maternally expressed Ube3a, Igf2r, Meg3/Gtl2 and the paternally expressed impact and U2af1-rs1 genes (FIG. 11), suggesting relaxation of imprinting for these genes.

TABLE 5
Identification of imprinted genes expressed in CD3+ splenocytes
B6129-1: sample from sorted CD3+ splenocytes from B6129 mouse (GFP transgenic)
Array Type Mouse430_2 (see supplementary Material and Methods)
Annotaton contains imprinted/or known imprinted genes

| File | B6129-1.TXT | | | | | | | | |
|---|---|---|---|---|---|---|---|---|---|
| Name ( )* | Normalized | | | | | | | Gene | |
| Systematic | Flags | | Raw | Common | Genbank | Map (cM) | | Symbol_Affym | Description |
| 1429257_at | 1.188147 | A | 81.1 | Gtl2 | AU067739 | | | Gtl2 | GTL2, imprinted maternally |
| | | | | | | | | | expressed untranslated mRNA |
| 1429256_at | 1 | P | 123.8 | Gtl2 | AU067739 | 12 54.0 | CM | Gtl2 | GTL2, imprinted maternally |
| | | | | | | | | | expressed untranslated mRNA |
| 1436057_at | 1.100959 | A | 72.3 | Gtl2 | BM117428 | 12 54.0 | cM | Gtl2 | GTL2, imprinted maternally |
| | | | | | | | | | expressed untranslated mRNA |
| 1452183_a_at | 1.004538 | A | 210.3 | Gtl2 | Y13832 | 12 54.0 | cM | Gtl2 | GTL2, imprinted maternally |
| | | | | | | | | | expressed untranslated mRNA |
| 1439380_x_at | 1.253889 | P | 247.7 | Gtl2 | BB093563 | 12 54.0 | cM | Gtl2 | BB093563 RIKEN full-length |
| | | | | | | | | | enriched, 12 days embryo, |
| | | | | | | | | | embryonic body between |
| | | | | | | | | | diaphragm region and neck |
| | | | | | | | | | Mus musculus cDNA clone |
| | | | | | | | | | 9430042P15 3′ similar to |
| | | | | | | | | | Y13832 Mus musculus mRNA for |
| | | | | | | | | | GT12 protein, mRNA sequence. |
| 1426758_s_at | 1 | A | 57.9 | Gtl2 | Y13832 | 12 54.0 | cM | Gtl2 | GTL2, imprinted maternally |
| | | | | | | | | | expressed untranslated mRNA |
| 1432297_at | 1.658693 | A | 18 | 1700116N | AK007205 | | | — | Adult male testis cDNA, RIKEN |
| | | | | | | | | | full-length enriched library, |
| | | | | | | | | | clone: 1700116N21 product: |
| | | | | | | | | | unclassifiable, full insert |
| | | | | | | | | | sequence |
| 1421968_a_at | 1 | P | 129.8 | 3830408P [symbol] | NM_023647 | | | 3830408P04Rik | RIKEN cDNA 3830408P04 gene |
| 1427678_at | 0.890432 | P | 68.6 | Zim3 | AF365932 | 7 7.0 | cM | — | Adult male testis cDNA, RIKEN |
| | | | | | | | | | full-length enriched library, |
| | | | | | | | | | clone: 1700128I23 product: |
| | | | | | | | | | zinc finger, imprinted 3, |
| | | | | | | | | | full insert sequence |
| 1446751_s_at | 1.437534 | A | 89.7 | E430016J: | BB524087 | | | Impact | imprinted and ancient |
| 1446750_at | 0.294425 | P | 1108 | E430016J: | BB524087 | | | Impact | imprinted and ancient |
| 1431229_at | 0.443792 | A | 4.3 | C030032C | AK019361 | 10 | C2 | C030032C09Rik | Mus musculus adult male |
| | | | | | | | | | hippocampus cDNA, RIKEN full- |
| | | | | | | | | | length enriched library, |
| | | | | | | | | | clone: 2900084A04 product: |
| | | | | | | | | | E2A-PBX1-ASSOCIATED PROTEIN |
| | | | | | | | | | (FRAGMENT) homolog [Homo sapiens], |
| | | | | | | | | | full insert sequence. |
| 1452899_at | 1.318152 | P | 59.9 | Rian | AK017440 | 12 54.5 | cM | — | 15 days embryo head cDNA, |
| | | | | | | | | | RIKEN full-length enriched |
| | | | | | | | | | library, clone: D930050K13 |
| | | | | | | | | | product: unclassifiable, full |
| | | | | | | | | | insert sequence |
| 1427580_a_at | 0.346807 | A | 17.7 | Rian | BB649603 | 12 54.5 | cM | — | 15 days embryo head cDNA, |
| | | | | | | | | | RIKEN full-length enriched |
| | | | | | | | | | library, clone: D930050K13 |
| | | | | | | | | | product: unclassifiable, full |
| | | | | | | | | | insert sequence |
| 1452905_at | 2.074902 | A | 57.9 | Gtl2 | AV015833 | 12 54.0 | cM | Gtl2 | GTL2, imprinted maternally |
| | | | | | | | | | expressed untranslated mRNA |
| 1428764_at | 1.135283 | A | 11 | Gtl2 | AV015833 | 12 54.0 | cM | Gtl2 | GTL2, imprinted maternally |
| | | | | | | | | | expressed untranslated mRNA |
| 1428765_at | 1.307113 | A | 47.6 | Gtl2 | AV015833 | 12 54.0 | cM | Gtl2 | GTL2, imprinted maternally |
| | | | | | | | | | expressed untranslated mRNA |
| 1452906_at | 1.178441 | A | 14.3 | Gtl2 | BE990468 | 12 54.0 | cM | Gtl2 | GTL2, imprinted maternally |
| | | | | | | | | | expressed untranslated mRNA |
| 1434864_at | 1 | P | 39 | Spg6 | BB326329 | | | A830014A18Rik | BB326329 RIKEN full-length |
| | | | | | | | | | enriched, 4 days neonate male |
| | | | | | | | | | adipose Mus musculus cDNA |
| | | | | | | | | | clone B430207K20 3′, mRNA |
| | | | | | | | | | sequence. |
| 1458598_at | 0.859959 | P | 128.5 | | BE979804 | | | — | Paternally expressed |
| | | | | | | | | | imprinted noncoding RNA short |
| | | | | | | | | | transcript (Peg13) mRNA, |
| | | | | | | | | | complete sequence |
| 1415911_at | 0.979517 | P | 275.9 | Impact | NM_008378 | | | Impact | imprinted and ancient |
| 1444767_at | 0.330264 | A | 3.2 | | AV253089 | | | — | 0 day neonate head cDNA, |
| | | | | | | | | | RIKEN full-length enriched |
| | | | | | | | | | library, clone: 4833445A15 |
| | | | | | | | | | product: hypothetical protein |
| | | | | | | | | | in the GNAS imprinted complex |
| | | | | | | | | | locus, full insert sequence |
| 1421405_at | 1.43611 | A | 15.1 | Zim1 | NM_011769 | 7 6.5 | cM | Zim1 | zinc finger, imprinted 1 |
| 1424079_x_at | 1 | A | 26.4 | 2900073H | BC026994 | | | 2900073H19Rik | RIKEN cDNA 2900073H19 gene |
| 1424111_at | 1.389385 | P | 202.2 | Igf2r | BG092290 | 17 7.35 | cM | Igf2r | insulin-like growth factor 2 |
| | | | | | | | | | receptor |
| 1424112_at | 2.696766 | P | 400.2 | Igf2r | BG092290 | 17 7.35 | cM | Igf2r | insulin-like growth factor 2 |
| | | | | | | | | | receptor |
| 1427394_at | 0.924973 | A | 38 | Igf2as | AB030734 | 7 69.09 | cM | — | Peg8/Igf2as mRNA, imprinting |
| | | | | | | | | | gene. |
| 1448152_at | 1.374398 | A | 53.8 | Igf2 | NM_010514 | 7 69.09 | cM | Igf2 | insulin-like growth factor 2 |
| 1415895_at | 1 | A | 1361 | Snrpn | NM_013670 | 7 29.0 | cM | Snrpn | small nuclear |
| | | | | | | | | | ribonucleoprotein N |
| 1415896_x_at | 0.853952 | A | 125.7 | Snrpn | NM_013670 | 7 29.0 | cM | Snrpn | small nuclear |
| | | | | | | | | | ribonucleoprotein N |
| 1417649_at | 2.524499 | P | 86.9 | Cdkn1c | NM_009876 | 7 69.49 | cM | Cdkn1c | cyclin-dependent kinase |
| | | | | | | | | | inhibitor 1C (P57) |
| 1436057_at | 1.100959 | A | 72.3 | Gtl2 | BM117428 | 12 54.0 | cM | Gtl2 | GTL2, imprinted maternally |
| | | | | | | | | | expressed untranslated mRNA |
| 1427678_at | 0.890432 | P | 68.6 | Zim3 | AF365932 | 7 7.0 | cM | — | Adult male testis cDNA, RIKEN |
| | | | | | | | | | full-length enriched library, |
| | | | | | | | | | clone: 1700128I23 product: |
| | | | | | | | | | zinc finger, imprinted 3, |
| | | | | | | | | | full insert sequence |
| 1449939_s_at | 0.765612 | P | 61.2 | Dlk1 | NM_010052 | 12 54.0 | cM | Dlk1 | delta-like 1 homolog |
| | | | | | | | | | (Drosophila) |
| 1423294_at | 1 | A | 52.3 | Mest | AW555393 | 6 7.5 | cM | — | Transcribed sequence with |
| | | | | | | | | | moderate similarity to protein |
| | | | | | | | | | sp: Q9UBF2 (H. sapiens) |
| | | | | | | | | | CPG2_HUMAN Coatomer gamma-2 |
| | | | | | | | | | subunit |
| 1416680_at | 1.704538 | P | 1083 | Ube3a | AK018443 | 7 28.65 | cM | Ube3a | ubiquitin protein ligase E3A |

Of the imprinted genes with Flag P = present, the following were chosen for RT-PCR analysis:
Gtl2 GTL2, imprinted maternally expressed untranslated mRNA
Impact imprinted and ancient
Igf2r insulin-like growth factor 2 receptor
Dlk1 delta-like 1 homolog (Drosophila)
Ube3a ubiquitin protein ligase E3A

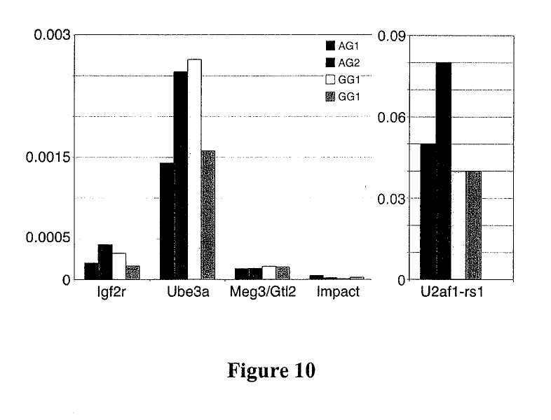

To analyze imprinted gene expression of engrafted tissue in reconstituted adults, we isolated uniparental-derived hematopoietic cells from adult recipients using FACS sorted (GFP, CD3 positive) splenocytes as a representative cell type and performed array analysis with the Affymetrix MOE 430A v2 mouse gene array Methods are provided by the Microarray Core Facility University of Pennsylvania website at med.upenn.edu/microarr/Data%20Analysis/Affymetrix/methods.htm. Expression of both maternally and paternally imprinted genes was detected in both AG and GG derived CD3+ splenocytes, respectively, indicating that in both types of uniparental cells, normally silent alleles had become active (FIG. 10, both maternally and paternally imprinted genes, see legend). Some imprinted genes exhibit tissue specific imprinting in adults, for instance, Igf2R, which is maternally expressed (paternally imprinted) in most tissues, including the spleen, but exhibits biallelic expression in the central nervous system (Hu et al., 1998). For the paternally expressed Peg1 gene monoallelic expression was observed in adult spleen (Reule et al., 1998), however, in interspecies hybrid mice, occasional loss of imprinting was reported. For the majority of the genes listed in FIG. 10, however, it is unknown if allele-specific expression is maintained in the normal adult spleen. Parent-of origin specific expression needs to be established/confirmed using interspecies and interstrain hybrid mice. For several genes, array results were confirmed by real-time RT-PCR (FIG. 11). Expression levels were low in comparison to β-actin but comparable to those detected in cells of normal ES cell origin. The similarity in expression level may indicate dosage compensation, since for maternally and paternally imprinted genes, based on an expectation of parent-specific monoallelic expression either an increase or lack of transcript would be expected in AG and GG cells

Sample Collection to Analyze Imprinted Gene Expression in Uniparental Transplants and Reconstituted Tissues.

The phenotype of uniparental chimeras, particularly AG, and the differentiation bias observed for AG and GG ES cells in teratomas are consistent with the ES cells maintaining their imprinting status and conferring imprinting based phenotypes prior to transplantation. Thus, non-allele specific gene expression in uniparental cells in the adults may indicate that there is a change in the status of imprinting of uniparental cells during the engraftment process.

To ascertain imprinted gene expression in uniparental cells at stages of engraftment cells at various stages of the transplantation/engraftment procedure will be collected. We have already collected ES cell derived (GFP positive) fetal liver cells from AG, GG and N chimeras, as well as from GFP-transgenic non-ES cell derived fetuses. Due to the high content of erythroid cells in the fetal liver, (which do not express GFP), the percentage of GFP positive fetal liver cells of transgenic B6Osb fetuses is only approximately 5-8% of all cells, and proportionally lower in chimeric fetal liver derived from injection of GFP-transgenic ES cells (AG, GG, N). By FACS sorting, we collected GFP positive cells from GFP-transgenic, N, GG and AG chimeras. The percentage of GFP positive cells in ES cell chimeric fetal livers ranged from 0.5% in medium to 8% in strong chimeras. Depending on the size of the fetal liver and the percentage of ES cell contribution, we collected between 17,000 and 250,000 GFP positive cells from individual day 13.5 to 14.5 fetal livers. From these cells, 120 to 670 ng of total RNA were isolated using an efficient method for the preparation of RNA from small samples. Briefly, flow sorted cells were collected into Trizol LS, and nucleic acids extracted using Qiagen RNeasy columns. This process provided sufficient starting material for array analysis with double amplification of the RNA target.

Methylation of Imprinting Control Regions in Uniparental Derived Tissue in Adults

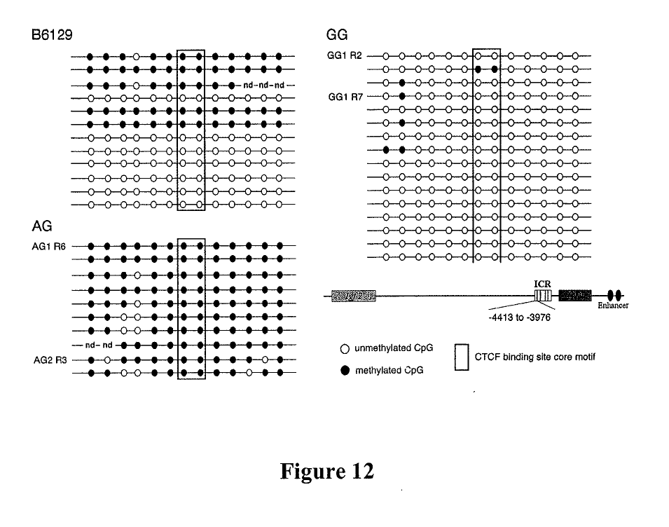

Bone marrow reconstituted entirely from uniparental transplants was obtained and nucleic acids isolated and subjected to bisulfite sequencing performed to determine methylation of cytosines in CpG islands in the 5′ upstream region of the H19 gene. This region is part of the imprinting control region that regulates reciprocal allele-specific expression of the H19 and Igf2 genes. In normal tissues, the paternal allele is methylated and the maternal allele non-methylated. Our preliminary data indicate that parent-of origin-specific methylation of this region is retained in uniparental derived bone marrow in reconstituted recipients: Clones derived from AG tissue exhibit a high degree of methylation, whereas clones from GG derived tissue are not methylated in this region (FIG. 12). These preliminary results suggest that parent-of-origin specific epigenetic marks are retained in uniparental cells that have engrafted in adult recipients.

ES Cell Lines and Mouse Strains Available in Laboratory

We are using the following mouse strains that are either ordered from vendors or maintained as breeding colonies in the Myrin Barrier Facility:

TABLE 6
Mouse strains available

| Strain | Abbreviated | Resource Order No. | Reference |
|---|---|---|---|
| C57BL/6NTac | B6 | Taconic# B6 | |
| C57BL/6-TgN | B6Osb | Jackson# 003291 | (Okabe et |
| (ACTbEGFP)1Osb | | | al., 1997) |
| 129S1/SvImJ | 129S1 | Jackson# 002448 | |
| 129S6/SvEv | 129Sv | Taconic# 129SVE | |
| B6129F1/Tac | B6129Sv | Taconic# B6129 | |
| (B6129 Hybrid) | | | |
| JF1/Ms (M. musculus | JF | Jackson# 003720 | (Koide et |
| molossinus) | | | al., 1998) |

In addition to the ES cell lines characterized previously we have the following ES cell lines available (all lines have normal chromosome number and have been sexed by PCR):

TABLE 7
ES cell lines available

| | Mouse strain | No. of lines |
|---|---|---|
| ES cell type | background | available |
| N (normal) | 129Sv | 10 |
| AG | 129S1 | 2 (previously |
| | | published*) |
| | 129Sv | 3 |
| N (JF hybrid) | B6C3xJF F1 | 2 |

*(McLaughlin et al., 1997)

Determination of Timing of Modulation of Gene Expression Due to Imprinting in Transplanted Uniparental Tissues by Assessing Imprinted Gene Expression and Methylation in Tissues Prior and Post Transplantation.

The incapacity of uniparental cells to proliferate equivalently into all tissue types and form normal embryos is associated with, and a consequence of, the over expression or lack of, imprinted genes that are normally expressed from either only the maternal or paternal allele. We observed that uniparental chimeras successfully used for hematopoietic transplants displayed imprinting-related phenotypes, suggesting that the uniparental cells retained their imprinting at fetal stages, prior to transplantation. In contrast, lymphocytes isolated from reconstituted adult recipients unexpectedly expressed imprinted genes at similar levels regardless of whether these cells originated from androgenetic, gynogenetic or normal transplants (see Example 1). Normal hematopoiesis was observed in adult recipients receiving transplants, irrespective of uniparental or normal origin.

The success of engraftment and the observed expression profile in reconstituted tissue suggests that, in reconstituted hematopoietic tissue within adult recipients, expression of a number of imprinted genes is regulated in a non parent-of-origin specific manner. This may reflect a possible mechanism that would permit engraftment of uniparental cells into various tissues by regulating normal levels of expression of imprinted genes in uniparental cells during or subsequent to engraftment. Alternatively, this finding may be the consequence of the selection of a subpopulation of cells exhibiting normal expression prior to engraftment. To ascertain how imprinting relates to engraftment and the function of uniparental cells in adult tissue, we will therefore characterize methylation and expression of imprinted genes in uniparental-derived tissue at various stages of the transplantation process. This analysis also addresses the requirement for parental allele specific regulation of imprinted genes in the adult. As a comparison, we will include adult uniparental chimeras (GG and N only) in which uniparental cells have co-developed with normal cells.

TABLE 8
Summary of preliminary observations of imprinting/phenotypes
in uniparental tissues

| | | Differ- | | |
|---|---|---|---|---|
| | undiffer- | entiated | | uniparental |
| | entiated | uniparental | fetal | tissue in |
| | uniparental | ES cells | uniparental | adult |
| | ES cells | (teratomas) | chimeras | recipient |
| uniparental | N/A | Yes | yes for AG | N/A |
| phenotype | | | | |
| allele- | n.d. | n.d. | n.d. | no for AG, |
| specific | | | | GG in hemato- |
| expression | | | | poietic cells |
| of imprinted | | | | |
| genes | | | | |
| allele- | n.d. | n.d. | n.d. | yes for AG, |
| specific | | | | GG in bone |
| methylation | | | | marrow |

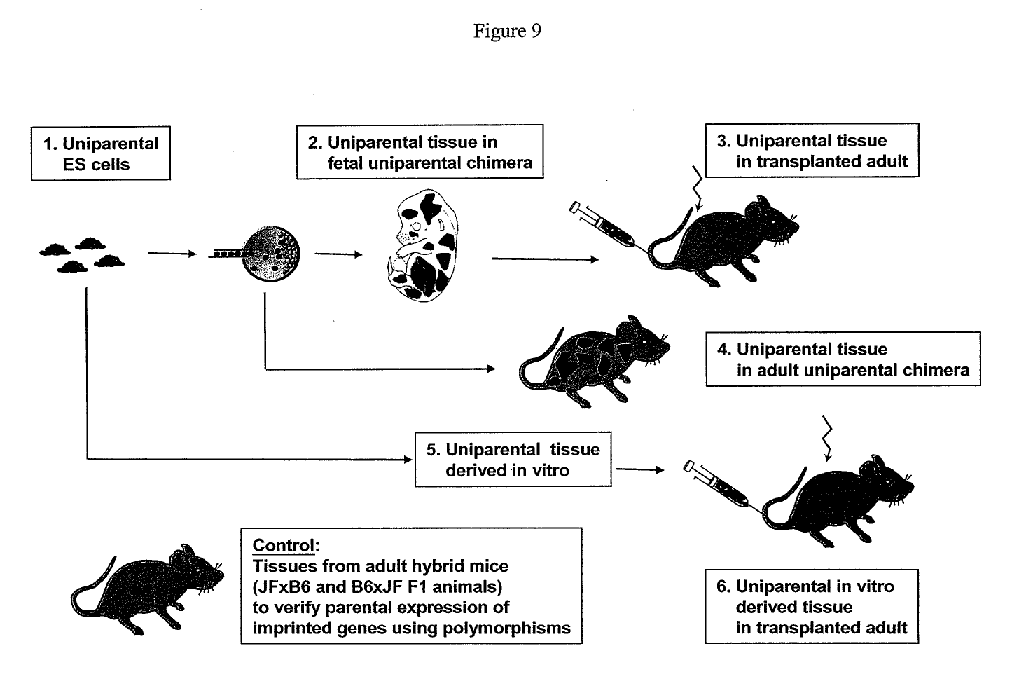

FIG. 9 illustrates the overall experimental design. For the generation of uniparental and normal (control) chimeras, we will use established GFP-transgenic uniparental ES cells (Table 3 AG ES lines 1 and 2, GG ES line 1, N ES line 1) as well as one additional GG and N ES line that will be derived as described herein. Imprinted gene expression and methylation of characterized and well-studied control regions of imprinted genes will be analyzed in uniparental cells/tissues prior to, and subsequent to transplantation into adults, as well as in uniparental chimeras. Tissues for analysis are numbered (1-6; FIG. 9), and tools for and detail on the analysis of each respective tissue are provided.

### REFERENCES

1. Kaufman, M. H., Barton, S. C. & Surani, M. A. Normal postimplantation development of mouse parthenogenetic embryos to the forelimb bud stage. Nature 265, 53-5. (1977).
2. McGrath, J. & Solter, D. Completion of mouse embryogenesis requires both the maternal and paternal genomes. Cell 37, 179-83 (1984).
3. Barton, S. C., Surani, M. A. & Norris, M. L. Role of paternal and maternal genomes in mouse development. Nature 311, 374-6. (1984).
4. Robertson, E. J., Kaufman, M. H., Bradley, A. & Evans, M. J. Isolation, properties, and karyotype analysis of pluripotential (EK) cell lines from normal and parthenogenetic embryos. in Teratocarcinomal Stem Cells. Cold Spring Harbor Conferences on Cell Proliferation. Vol. 10 (eds Silver, L. M., Martin, G. R. & Strickland, S.) 647-663 (Cold Spring Harbor Laboratory, New York, 1983).
5. Mann, J. R., Gadi, I., Harbison, M. L., Abbondanzo, S. J. & Stewart, C. L. Androgenetic mouse embryonic stem cells are pluripotent and cause skeletal defects in chimeras: Implications for genetic imprinting. Cell 62, 251-260 (1990).
6. Cibelli, J. B. et al. Parthenogenetic stem cells in nonhuman primates. Science 295, 819. (2002).
7. Surani, M. A., Barton, S. C. & Kaufman, M. H. Development to term of chimaeras between diploid parthenogenetic and fertilised embryos. Nature 270, 601-3. (1977).
8. Stevens, L. C. Totipotent cells of parthenogenetic origin in a chimaeric mouse. Nature 276, 266-7. (1978).
9. Surani, M. A., Barton, S. C., Howlett, S. K. & Norris, M. L. Influence of chromosomal determinants on development of androgenetic and parthenogenetic cells. Development 103, 171-8. (1988).
10. Fundele, R., Norris, M. L., Barton, S. C., Reik, W. & Surani, M. A. Systematic elimination of parthenogenetic cells in mouse chimeras. Development 106, 29-35. (1989).
11. Nagy, A., Sass, M. & Markkula, M. Systematic non-uniform distribution of parthenogenetic cells in adult mouse chimaeras. Development 106, 321-4. (1989).
12. Allen, N. D., Barton, S. C., Hilton, K., Norris, M. L. & Surani, M. A. A functional analysis of imprinting in parthenogenetic embryonic stem cells. Development 120, 1473-82 (1994).
13. McLaughlin, K. J. et al. Roles of the imprinted gene Igf2 and paternal duplication of distal chromosome 7 in the perinatal abnormalities of androgenetic mouse chimeras. Development 124, 4897-904. (1997).
14. Surani, M. A. & Barton, S. C. Development of gynogenetic eggs in the mouse: implications for parthenogenetic embryos. Science 222, 1034-6. (1983).
15. Barton, S. C., Ferguson-Smith, A. C., Fundele, R. & Surani, M. A. Influence of paternally imprinted genes on development. Development 113, 679-87. (1991).
16. Obata, Y. et al. Post-implantation development of mouse androgenetic embryos produced by in-vitro fertilization of enucleated oocytes. Hum Reprod 15, 874-80. (2000).
17. Lagutina, I., Lazzari, G., Duchi, R. & Galli, C. Developmental potential of bovine androgenetic and parthenogenetic embryos: a comparative study. Biol Reprod 70, 400-5 (2004).
18. Hernandez, L., Kozlov, S., Piras, G. & Stewart, C. L. Paternal and maternal genomes confer opposite effects on proliferation, cell-cycle length, senescence, and tumor formation. Proc Natl Acad Sci USA 100, 13344-9 (2003).
19. Jagerbauer, E. M., Fraser, A., Herbst, E. W., Kothary, R. & Fundele, R. Parthenogenetic stem cells in postnatal mouse chimeras. Development 116, 95-102. (1992).
20. Morrison, S. J., Hemmati, H. D., Wandycz, A. M. & Weissman, I. L. The purification and characterization of fetal liver hematopoietic stem cells. Proc Natl Acad Sci USA 92, 10302-6. (1995).
21. Okabe, M., Ikawa, M., Kominami, K., Nakanishi, T. & Nishimune, Y. ‘Green mice’ as a source of ubiquitous green cells. FEBS Lett 407, 313-9. (1997).
22. Szabo, P. & Mann, J. R. Expression and methylation of imprinted genes during in vitro differentiation of mouse parthenogenetic and androgenetic embryonic stem cell lines. Development 120, 1651-60 (1994).
23. Sturm, K. S. et al. Unrestricted lineage differentiation of parthenogenetic ES cells. Dev Genes Evol 206, 377-388 (1997).
25. Narasimha, M., Barton, S. C. & Surani, M. A. The role of the paternal genome in the development of the mouse germ line. Curr Biol 7, 881-4. (1997).
26. Strain, L., Warner, J. P., Johnston, T. & Bonthron, D. T. A human parthenogenetic chimera. Nat Genet 11, 164-9. (1995).
27. Kono, T. et al. Birth of parthenogenetic mice that can develop to adulthood. Nature 428, 860-4 (2004).
28. Zhang, C. C. & Lodish, H. F. Insulin-like growth factor 2 expressed in a novel fetal liver cell population is a growth factor for hematopoietic stem cells. Blood 103, 2513-21 (2004).
29. Forrester, L. M., Bernstein, A., Rossant, J. & Nagy, A. Long-term reconstitution of the mouse hematopoietic system by embryonic stem cell-derived fetal liver. Proc Natl Acad Sci USA 88, 7514-7. (1991).
30. Mann, M. R. et al. Disruption of imprinted gene methylation and expression in cloned preimplantation stage mouse embryos. Biol Reprod 69, 902-14 (2003).
31. Boediono, A., Suzuki, T., Li, L. Y. and Godke, R. A. (1999). Offspring born from chimeras reconstructed from parthenogenetic and in vitro fertilized bovine embryos. Mol Reprod Dev 53, 159-70.

### ADDITIONAL REFERENCES CITED BY AUTHOR NAME

Hooper, M., Hardy, K., Handyside, A., Hunter, S. & Monk, M. HPRT-deficient (Lesch-Nyhan) mouse embryos derived from germline colonization by cultured cells. Nature 326, 292-5 (1987).
McLaughlin, K. J. et al. Roles of the imprinted gene Igf2 and paternal duplication of distal chromosome 7 in the perinatal abnormalities of androgenetic mouse chimeras. Development 124, 4897-904. (1997).
Abbondanzo, S. J., Gadi, I. and Stewart, C. L. (1993). Derivation of embryonic stem cell lines. Methods Enzymol 225, 803-23.
Kaufman, D. S. and Thomson, J. A. (2002). Human ES cells—haematopoiesis and transplantation strategies. J Anat 200, 243-8.
Keller, G., Kennedy, M., Papayannopoulou, T. and Wiles, M. V. (1993). Hematopoietic commitment during embryonic stem cell differentiation in culture. Mol Cell Biol 13, 473-86.
Kyba, M., Perlingeiro, R. C. and Daley, G. Q. (2002). HoxB4 confers definitive lymphoid-myeloid engraftment potential on embryonic stem cell and yolk sac hematopoietic progenitors. Cell 109, 29-37.
Mann, J. R. (1992). Properties of androgenetic and parthenogenetic mouse embryonic stem cell lines; are genetic imprints conserved? Seminars Dev. Biol. 3, 77-85.
Morali, O. G., Jouneau, A., McLaughlin, K. J., Thiery, J. P. and Larue, L. (2000). IGF-II promotes mesoderm formation. Dev Biol 227, 133-45.
Mummery, C., Ward, D., van den Brink, C. E., Bird, S. D., Doevendans, P. A., Opthof, T., Brutel de la Riviere, A., Tertoolen, L., van der Heyden, M. and Pera, M. (2002). Cardiomyocyte differentiation of mouse and human embryonic stem cells. J Anat 200, 233-42.
Shufaro, Y. and Reubinoff, B. E. (2004). Therapeutic applications of embryonic stem cells. Best Pract Res Clin Obstet Gynaecol 18, 909-27.
Stevens, L. C., Varnum, D. S. and Eicher, E. M. (1977). Viable chimaeras produced from normal and parthenogenetic mouse embryos. Nature 269, 515-7.
Strain, L., Warner, J. P., Johnston, T. and Bonthron, D. T. (1995). A human parthenogenetic chimera. Nat Genet 11, 164-9.
Thomson, J. A., Itskovitz-Eldor, J., Shapiro, S. S., Waknitz, M. A., Swiergiel, J. J., Marshall, V. S. and Jones, J. M. (1998). Embryonic stem cell lines derived from human blastocysts. Science 282, 1145-7.
Trounson, A. (2002). Human embryonic stem cells: mother of all cell and tissue types. Reprod Biomed Online 4, 58-63.
Wang, L., Menendez, P., Cerdan, C. and Bhatia, M. (2005a). Hematopoietic development from human embryonic stem cell lines. Exp Hematol 33, 987-96.
Wang, L., Menendez, P., Shojaei, F., Li, L., Mazurier, F., Dick, J. E., Cerdan, C., Levac, K. and Bhatia, M. (2005b). Generation of hematopoietic repopulating cells from human embryonic stem cells independent of ectopic HOXB4 expression. J Exp Med 201, 1603-14.
Ishii T, Yasuchika K, Fujii H, Hoppo T, Baba S, Naito M, Machimoto T, Kamo N, Suemori H, Nakatsuji N, Ikai I. In vitro differentiation and maturation of mouse embryonic stem cells into hepatocytes. Exp Cell Res. 2005 Sep. 10; 309(1):68-77.
Kobayashi T, Tanaka H, Kuwana H, Inoshita S, Teraoka H, Sasaki S, Terada Y. Wnt4-transformed mouse embryonic stem cells differentiate into renal tubular cells. Biochem Biophys Res Commun. 2005 Oct. 21; 336(2):585-95.
McCloskey K E, Gilroy M E, Nerem R M. Use of embryonic stem cell-derived endothelial cells as a cell source to generate vessel structures in vitro. Tissue Eng. 2005 March-April; 11(3-4):497-505
Liersch R, Nay F, Lu L, Detmar M. Induction of lymphatic endothelial cell differentiation in embryoid bodies. Blood. 2005 Sep. 29; [Epub ahead of print]
Liour S S, Kraemer S A, Dinkins M B, Su C Y, Yanagisawa M, Yu R K. Further characterization of embryonic stem cell-derived radial glial cells. Glia. 2005 Sep. 12; [Epub ahead of print]
Schroeder M, Niebruegge S, Werner A, Willbold E, Burg M, Ruediger M, Field L J, Lehmann J, Zweigerdt R. Differentiation and lineage selection of mouse embryonic stem cells in a stirred bench scale bioreactor with automated process control. Biotechnol Bioeng. 2005 Sep. 27; [Epub ahead of print]
Barberi, T., Klivenyi, P., Calingasan, N. Y., Lee, H., Kawamata, H., Loonam, K., Perrier, A. L., Bruses, J., Rubio, M. E., Topf, N. et al. (2003). Neural subtype specification of fertilization and nuclear transfer embryonic stem cells and application in parkinsonian mice. Nat Biotechnol 21, 1200-7.
Barberi, T., Willis, L. M., Socci, N. D. and Studer, L. (2005). Derivation of multipotent mesenchymal precursors from human embryonic stem cells. PLoS Med 2, e161.
Blyszczuk, P., Czyz, J., Kania, G., Wagner, M., Roll, U., St-Onge, L. and Wobus, A. M. (2003). Expression of Pax4 in embryonic stem cells promotes differentiation of nestin-positive progenitor and insulin-producing cells. Proc Natl Acad Sci USA 100, 998-1003.
Blyszczuk, P. and Wobus, A. M. (2004). Stem cells and pancreatic differentiation in vitro. J Biotechnol 113, 3-13.
Boheler, K. R., Czyz, J., Tweedie, D., Yang, H. T., Anisimov, S. V. and Wobus, A. M. (2002). Differentiation of pluripotent embryonic stem cells into cardiomyocytes. Circ Res 91, 189-201.
Capi, O. and Gepstein, L. (2006). Myocardial regeneration strategies using human embryonic stem cell-derived cardiomyocytes. J Control Release 116, 211-8.
Caspi, O., Lesman, A., Basevitch, Y., Gepstein, A., Arbel, G., Habib, I. H., Gepstein, L. and Levenberg, S. (2007). Tissue engineering of vascularized cardiac muscle from human embryonic stem cells. Circ Res 100, 263-72.
Choi, K., Kennedy, M., Kazarov, A., Papadimitriou, J. C. and Keller, G. (1998). A common precursor for hematopoietic and endothelial cells. Development 125, 725-32.
Cibelli, J. B., Grant, K. A., Chapman, K. B., Cunniff, K., Worst, T., Green, H. L., Walker, S. J., Gutin, P. H., Vilner, L., Tabar, V. et al. (2002). Parthenogenetic stem cells in nonhuman primates. Science 295, 819.
Du, Z. W. and Zhang, S. C. (2004). Neural differentiation from embryonic stem cells: which way? Stem Cells Dev 13, 372-81.
Feraud, O. and Vittet, D. (2003). Murine embryonic stem cell in vitro differentiation: applications to the study of vascular development. Histol Histopathol 18, 191-9.
Fukuda, K. and Yuasa, S. (2006). Stem cells as a source of regenerative cardiomyocytes. Circ Res 98, 1002-13.
Gangaram-Panday, S. T., Faas, M. M. and de Vos, P. (2007). Towards stem-cell therapy in the endocrine pancreas. Trends Mol Med.
Hansson, M., Tonning, A., Frandsen, U., Petri, A., Rajagopal, J., Englund, M. C., Heller, R. S., Hakansson, J., Fleckner, J., Skold, H. N. et al. (2004). Artifactual insulin release from differentiated embryonic stem cells. Diabetes 53, 2603-9.
Haruta, M. (2005). Embryonic stem cells: potential source for ocular repair. Semin Opthalmol 20, 17-23.
Heo, J., Factor, V. M., Uren, T., Takahama, Y., Lee, J. S., Major, M., Feinstone, S. M. and Thorgeirsson, S. S. (2006). Hepatic precursors derived from murine embryonic stem cells contribute to regeneration of injured liver. Hepatology 44, 1478-86.
Hwang, N. S., Kim, M. S., Sampattavanich, S., Baek, J. H., Zhang, Z. and Elisseeff, J. (2006). Effects of three-dimensional culture and growth factors on the chondrogenic differentiation of murine embryonic stem cells. Stem Cells 24, 284-91.
Kaufman, D. S., Hanson, E. T., Lewis, R. L., Auerbach, R. and Thomson, J. A. (2001). Hematopoietic colony-forming cells derived from human embryonic stem cells. Proc Natl Acad Sci USA 98, 10716-21.
Kawaguchi, J., Mee, P. J. and Smith, A. G. (2005). Osteogenic and chondrogenic differentiation of embryonic stem cells in response to specific growth factors. Bone 36, 758-69.
Kawai, T., Takahashi, T., Esaki, M., Ushikoshi, H., Nagano, S., Fujiwara, H. and Kosai, K. (2004). Efficient cardiomyogenic differentiation of embryonic stem cell by fibroblast growth factor 2 and bone morphogenetic protein 2. Circ J 68, 691-702.
Kawasaki, H., Mizuseki, K., Nishikawa, S., Kaneko, S., Kuwana, Y., Nakanishi, S., Nishikawa, S. I. and Sasai, Y. (2000). Induction of midbrain dopaminergic neurons from ES cells by stromal cell-derived inducing activity. Neuron 28, 31-40.
Keller, G., Kennedy, M., Papayannopoulou, T. and Wiles, M. V. (1993). Hematopoietic commitment during embryonic stem cell differentiation in culture. Mol Cell Biol 13, 473-86.
Kennedy, M. and Keller, G. M. (2003). Hematopoietic commitment of ES cells in culture. Methods Enzymol 365, 39-59.
Kim, J. H., Auerbach, J. M., Rodriguez-Gomez, J. A., Velasco, I., Gavin, D., Lumelsky, N., Lee, S. H., Nguyen, J., Sanchez-Pemaute, R., Bankiewicz, K. et al. (2002). Dopamine neurons derived from embryonic stem cells function in an animal model of Parkinson's disease. Nature 418, 50-6.
Kim, J. Y., Koh, H. C., Lee, J. Y., Chang, M. Y., Kim, Y. C., Chung, H. Y., Son, H., Lee, Y. S., Studer, L., McKay, R. et al. (2003). Dopaminergic neuronal differentiation from rat embryonic neural precursors by Nurr1 overexpression. J. Neurochem 85, 1443-54.
Kramer, J., Hargus, G. and Rohwedel, J. (2006). Derivation and characterization of chondrocytes from embryonic stem cells in vitro. Methods Mol Biol 330, 171-90.
Kramer, J., Hegert, C. and Rohwedel, J. (2003). In vitro differentiation of mouse ES cells: bone and cartilage. Methods Enzymol 365, 251-68. Lee, S. H., Lumelsky, N., Studer, L., Auerbach, J. M. and McKay, R. D. (2000). Efficient generation of midbrain and hindbrain neurons from mouse embryonic stem cells. Nat Biotechnol 18, 675-9.
Levenberg, S., Golub, J. S., Amit, M., Itskovitz-Eldor, J. and Langer, R. (2002). Endothelial cells derived from human embryonic stem cells. Proc Natl Acad Sci USA 99, 4391-6.
Lumelsky, N., Blondel, O., Laeng, P., Velasco, I., Ravin, R. and McKay, R. (2001). Differentiation of embryonic stem cells to insulin-secreting structures similar to pancreatic islets. Science 292, 1389-94.
Miyazaki, S., Yamato, E. and Miyazaki, J. (2004). Regulated expression of pdx-1 promotes in vitro differentiation of insulin-producing cells from embryonic stem cells. Diabetes 53, 1030-7.
Olsen, A. L., Stachura, D. L. and Weiss, M. J. (2006). Designer blood: creating hematopoietic lineages from embryonic stem cells. Blood 107, 1265-75.
Rajagopal, J., Anderson, W. J., Kume, S., Martinez, O. I. and Melton, D. A. (2003). Insulin staining of ES cell progeny from insulin uptake. Science 299, 363.
Salero, E. and Hatten, M. E. (2007). Differentiation of ES cells into cerebellar neurons. Proc Natl Acad Sci USA 104, 2997-3002.
Sipione, S., Eshpeter, A., Lyon, J. G., Korbutt, G. S. and Bleackley, R. C. (2004). Insulin expressing cells from differentiated embryonic stem cells are not beta cells. Diabetologia 47, 499-508.
Sonntag, K. C. and Sanchez-Pernaute, R. (2006). Tailoring human embryonic stem cells for neurodegenerative disease therapy. Curr Opin Investig Drugs 7, 614-8.
Soria, B., Roche, E., Berna, G., Leon-Quinto, T., Reig, J. A. and Martin, F. (2000). Insulin-secreting cells derived from embryonic stem cells normalize glycemia in streptozotocin-induced diabetic mice. Diabetes 49, 157-62.
Soto-Gutierrez, A., Kobayashi, N., Rivas-Carrillo, J. D., Navarro-Alvarez, N., Zhao, D., Okitsu, T., Noguchi, H., Basma, H., Tabata, Y., Chen, Y. et al. (2006). Reversal of mouse hepatic failure using an implanted liver-assist device containing ES cell-derived hepatocytes. Nat Biotechnol 24, 1412-9.
Tian, X., Woll, P. S., Morris, J. K., Linehan, J. L. and Kaufman, D. S. (2006). Hematopoietic engraftment of human embryonic stem cell-derived cells is regulated by recipient innate immunity. Stem Cells 24, 1370-80.
Trounson, A. (2007). Use of embryonic stem cells for endocrine disorders. Horm Res 67 Suppl 1, 28-31.
Wang, L., Menendez, P., Shojaei, F., Li, L., Mazurier, F., Dick, J. E., Cerdan, C., Levac, K. and Bhatia, M. (2005). Generation of hematopoietic repopulating cells from human embryonic stem cells independent of ectopic HOXB4 expression. J Exp Med 201, 1603-14.
Wang, Z. Z., Au, P., Chen, T., Shao, Y., Daheron, L. M., Bai, H., Arzigian, M., Fukumura, D., Jain, R. K. and Scadden, D. T. (2007). Endothelial cells derived from human embryonic stem cells form durable blood vessels in vivo. Nat Biotechnol 25, 317-8.
Yamashita, J., Itoh, H., Hirashima, M., Ogawa, M., Nishikawa, S., Yurugi, T., Naito, M. and Nakao, K. (2000). Flk1-positive cells derived from embryonic stem cells serve as vascular progenitors. Nature 408, 92-6.
Zhao, X., Liu, J. and Ahmad, I. (2002). Differentiation of embryonic stem cells into retinal neurons. Biochem Biophys Res Commun 297, 177-84.
Zhou, X. and Gallicano, G. I. (2006). Microvascular tubes derived from embryonic stem cells sustain blood flow. Stem Cells Dev 15, 335-47.
Chen, S. C., Mullon, C., Kahaku, E., Watanabe, F., Hewitt, W., Eguchi, S., Middleton, Y., Arkadopoulos, N., Rozga, J., Solomon, B. et al. (1997). Treatment of severe liver failure with a bioartificial liver. Ann N Y Acad Sci 831, 350-60.
Dani, C., Smith, A. G., Dessolin, S., Leroy, P., Staccini, L., Villageois, P., Darimont, C. and Ailhaud, G. (1997). Differentiation of embryonic stem cells into adipocytes in vitro. J Cell Sci 110 (Pt 11), 1279-85.
De Sutter, P., Dozortsev, D., Cieslak, J., Wolf, G., Verlinsky, Y. and Dyban, A. (1992). Parthenogenetic activation of human oocytes by puromycin. J Assist Reprod Genet 9, 328-37.
Demetriou, A. A., Brown, R. S., Jr., Busuttil, R. W., Fair, J., McGuire, B. M., Rosenthal, P., Am Esch, J. S., 2nd, Lerut, J., Nyberg, S. L., Salizzoni, M. et al. (2004). Prospective, randomized, multicenter, controlled trial of a bioartificial liver in treating acute liver failure. Ann Surg 239, 660-7; discussion 667-70.
Geary, S. and Moon, Y. S. (2006). The human embryo in vitro: recent progress. J Reprod Med 51, 293-302.
Goldberg Cohen, I., Beck, G., Ziskind, A. and Itskovitz-Eldor, J. (2006). Human embryonic stem cells for vascular development and repair. Isr Med Assoc J 8, 573-8.
Keller, G. (2005). Embryonic stem cell differentiation: emergence of a new era in biology and medicine. Genes Dev 19, 1129-55.
Liew, C. G., Moore, H., Ruban, L., Shah, N., Cosgrove, K., Dunne, M. and Andrews, P. (2005). Human embryonic stem cells: possibilities for human cell transplantation. Ann Med 37, 521-32.
McGrath, J. and Solter, D. (1983). Nuclear transplantation in the mouse embryo by microsurgery and cell fusion. Science 220, 1300-2.
Menendez, P., Bueno, C. and Wang, L. (2006). Human embryonic stem cells: A journey beyond cell replacement therapies. Cytotherapy 8, 530-41.
Menendez, P., Bueno, C., Wang, L. and Bhatia, M. (2005). Human embryonic stem cells: potential tool for achieving immunotolerance? Stem Cell Rev 1, 151-8.
Nagy, Z. P. (2003). Micromanipulation of the human oocyte. Reprod Biomed Online 7, 634-40.
Olsen, A. L., Stachura, D. L. and Weiss, M. J. (2006). Designer blood: creating hematopoietic lineages from embryonic stem cells. Blood 107, 1265-75.
Palermo, G. D., Cohen, J. and Rosenwaks, Z. (1996). Intracytoplasmic sperm injection: a powerful tool to overcome fertilization failure. Fertil Steril 65, 899-908.
Payne, D. (1995a). Intracytoplasmic sperm injection: instrumentation and injection technique. Reprod Fertil Dev 7, 185-96.
Payne, D. (1995b). Micro-assisted fertilization. Reprod Fertil Dev 7, 831-9.
Pera, M. F., Filipczyk, A. A., Hawes, S. M. and Laslett, A. L. (2003). Isolation, characterization, and differentiation of human embryonic stem cells. Methods Enzymol 365, 429-46.
Rogers, N. T., Hobson, E., Pickering, S., Lai, F. A., Braude, P. and Swann, K. (2004). Phospholipase Czeta causes Ca2+ oscillations and parthenogenetic activation of human oocytes. Reproduction 128, 697-702.
Rosen, E. D., Sarraf, P., Troy, A. E., Bradwin, G., Moore, K., Milstone, D. S., Spiegelman, B. M. and Mortensen, R. M. (1999). PPAR gamma is required for the differentiation of adipose tissue in vivo and in vitro. Mol Cell 4, 611-7.
Shirahashi, H., Wu, J., Yamamoto, N., Catana, A., Wege, H., Wager, B., Okita, K. and Zern, M. A. (2004). Differentiation of human and mouse embryonic stem cells along a hepatocyte lineage. Cell Transplant 13, 197-211.
Brustle, O., Spiro, A. C., Karram, K., Choudhary, K., Okabe, S. and McKay, R. D. (1997). In vitro-generated neural precursors participate in mammalian brain development. Proc Natl Acad Sci USA 94, 14809-14.
Conti, L., Pollard, S. M., Gorba, T., Reitano, E., Toselli, M., Biella, G., Sun, Y., Sanzone, S., Ying, Q. L., Cattaneo, E. et al. (2005). Niche-independent symmetrical self-renewal of a mammalian tissue stem cell. PLoS Biol 3, e283.
Kirchhof, N., Harder, F., Petrovic, S., Kreutzfeldt, S., Schmittwolf, C., Durr, M., Kirsten, J., Muhl, B., Merkel, A. and Muller, A. M. (2002). Developmental potential of hematopoietic and neural stem cells: unique or all the same? Cells Tissues Organs 171, 77-89.
Schmittwolf, C., Kirchhof, N., Jauch, A., Durr, M., Harder, F., Zenke, M. and Muller, A. M. (2005). In vivo haematopoietic activity is induced in neurosphere cells by chromatin-modifying agents. Embo J 24, 554-66.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for the production of embryonic stem cells from cells derived from uniparental embryos for reconstitution of adult tissues or organs comprising:
a) producing a uniparental embryo;
b) culturing said embryo under conditions which result in the formation of a blastocyst;
c) isolating embryonic stem cells from said blastocyst;
d) exposing said cells to a receptor ligand cocktail which induces differentiation of said cells into a desired cell type;
e) culturing the cells of step d) for a suitable time period to generate an effective amount of cells of the desired cell type; and
f) optionally isolating the cells of step e).

2. The method as claimed in claim 1, wherein said uniparental embryo is selected from the group consisting of a parthenogenetic embryo, a gynogenetic embryo or an androgenetic embryo.

3. The method of claim 1, wherein said cell type is selected from the group consisting of hematopoietic cells, neuronal cells, cardiac myocytes, insulin producing cells, primordial germ cells, adipocytes, retinal cells, and hepatic cells.

4. The method of claim 2, wherein said embryo is an androgenetic embryo produced by a method selected from the group consisting of pronuclear transplantation between zygotes, double ICSI of enucleated oocytes and ICSI or IVF of enucleated oocytes followed by pronuclear transfer between haploid embryos to restore diploidy.

5. The method of claim 2, wherein said embryo is a parthenogenetic embryo prepared by activation of oocytes in the presence of cytoskeletal inhibitor to produce a diploid embryo.

6. The method of claim 2, wherein said embryo is a gynogenetic embryo prepared by activation of oocytes followed by pronuclear transplantation at the pronuclear stage to produce a diploid embryo.

7. The method of claim 1, further comprising introduction of a nucleic acid encoding a protein of interest into said ES cells.

8. The method of claim 1, wherein said uniparental embryos are of human origin.

9. The method of claim 1, wherein said uniparental embryos are of mammalian origin.

10. A method for reconstituting the hematopoietic system in a non-human mammal comprising:
a) providing a uniparental embryo;
b) culturing said embryo under conditions which result in the formation of a blastocyst;
c) plating zona-free blastocyst of b) on feeder fibroblasts;
d) deriving ES cells from outgrowths of said blastocyst;
e) injecting the ES cells derived in step d) into blastocysts thereby producing an ES cell chimera;
f) transferring said chimera into a pseudopregnant female;
g) recovering at least one fetus from said female;
h) obtaining a cell suspension from the liver of said chimeric fetus and injecting said cell suspension into an immunocompromised animal, said cells being capable of forming all cells of the hematopoietic lineage, thereby reconstituting the hematopoietic system in said immunocompromised animal.

11. The method of claim 8, wherein said immunocompromised animal has been subjected to lethal irradiation.

12. The method of claim 8, wherein said uniparental embryo is selected from the group consisting of a parthenogenetic embryo, a gynogenetic embryo or an androgenetic embryo.

13. The method of claim 1, wherein said uniparental embryos contain cells expressing a detectable label.

14. The method of claim 11, wherein said label is GFP.

15. A method for assaying modulation of gene expression due to imprinting comprising:
a) produce a uniparental embryo;
b) obtaining embryonic stem cells from said embryo and injecting said cells into a blastocyst, thereby creating a chimeric blastocyst;
c) transferring said blastocyst into pseudopregnant female;
d) optionally obtaining uniparental cells from said at least one fetus from said female and analyzing the cells therein for modulation of imprinted gene expression.

16. The method of claim 15 optionally further comprising assessing the methylation status of imprinted genes.

17. The method of claim 15, wherein said fetus develops post-natally and cells are harvested therefrom to assess modulation of imprinted gene expression.

18. The method of claim 17, optionally further comprising determination of status of imprinting by assessing alterations of levels of methylation of imprinted genes.

19. The method of claim 15, wherein said modulation of imprinted gene expression is determined via microarray analysis.

20. The method of claim 15, wherein said methylation status of said imprinted genes is determined via bisulfite sequencing.

21. A composition comprising the differentiated cells of claim 1 in a biologically acceptable carrier.

22. The composition of claim 21, further comprising a biologically acceptable polymer.

23. The composition of claim 21, which is macroencapsulated.

24. The composition of claim 21, which is microencapsulated.

25. The composition of claim 21, comprising a cell type selected from the group consisting of a hematopoietic cell, a neuronal cell, a cardiac myocyte, an insulin producing cell, an adipocyte, a retinal cell and a hepatic cell.


SEQUENCE LISTING (25 sequences)

SEQ ID NO: 1 - DNA, 20 bp, Artificial Sequence
  Synthetic Sequence
tgtgttctgctttcttggtg

SEQ ID NO: 2 - DNA, 20 bp, Artificial Sequence
  Synthetic Sequence
tagttgcagctctttgcacg

SEQ ID NO: 3 - DNA, 19 bp, Artificial Sequence
  Synthetic Sequence
acagctcaaacctgaagcg

SEQ ID NO: 4 - DNA, 21 bp, Artificial Sequence
  Synthetic Sequence
ttcagatctgacctcagaccc

SEQ ID NO: 5 - DNA, 18 bp, Artificial Sequence
  Synthetic Sequence
agttctcttgcgcttggc

SEQ ID NO: 6 - DNA, 20 bp, Artificial Sequence
  Synthetic Sequence
ttgcacatttcctgtgggac

SEQ ID NO: 7 - DNA, 20 bp, Artificial Sequence
  Synthetic Sequence
aagcaccatgagccactagg

SEQ ID NO: 8 - DNA, 22 bp, Artificial Sequence
  Synthetic Sequence
ctggcggtcaatatcatcttcc

SEQ ID NO: 9 - DNA, 23 bp, Artificial Sequence
  Synthetic Sequence
gaggaaggggttcttagatagcg

SEQ ID NO: 10 - DNA, 22 bp, Artificial Sequence
  Synthetic Sequence
ctaagacttggatcccagaacc

SEQ ID NO: 11 - DNA, 22 bp, Artificial Sequence
  Synthetic Sequence
gttcttctccttgggttctttc

SEQ ID NO: 12 - DNA, 20 bp, Artificial Sequence
  Synthetic Sequence
tagtcctgtgaaggtgtggg

SEQ ID NO: 13 - DNA, 20 bp, Artificial Sequence
  Synthetic Sequence
gtagggatgggttgatttgg

SEQ ID NO: 14 - DNA, 20 bp, Artificial Sequence
  Synthetic Sequence
cacatatgatgaagctacga

SEQ ID NO: 15 - DNA, 19 bp, Artificial Sequence
  Synthetic Sequence
cacactcccttcatattcc

SEQ ID NO: 16 - DNA, 20 bp, Artificial Sequence
  Synthetic Sequence
acgtttccccattttacaag

SEQ ID NO: 17 - DNA, 22 bp, Artificial Sequence
  Synthetic Sequence
ctctacatatgattttctctac

SEQ ID NO: 18 - DNA, 20 bp, Artificial Sequence
  Synthetic Sequence
taaggcagcaccacttggac

SEQ ID NO: 19 - DNA, 20 bp, Artificial Sequence
  Synthetic Sequence
taaggcagcaccacttggac

SEQ ID NO: 20 - DNA, 21 bp, Artificial Sequence
  Synthetic Sequence
gatatcgctgcgctggtcgtc

SEQ ID NO: 21 - DNA, 25 bp, Artificial Sequence
  Synthetic Sequence
acgcagctcattgtagaaggtgtgg

SEQ ID NO: 22 - DNA, 20 bp, Artificial Sequence
  Synthetic Sequence
ctcatgctgggactttgtgt

SEQ ID NO: 23 - DNA, 20 bp, Artificial Sequence
  Synthetic Sequence
ttttgaattattataaggaa

SEQ ID NO: 24 - DNA, 20 bp, Artificial Sequence
  Synthetic Sequence
atcaaatatcctcataaata

SEQ ID NO: 25 - DNA, 20 bp, Artificial Sequence
  Synthetic Sequence
acaaccctaatctttacaca